(19) United States
(12) Patent Application Publication
(10) Pub. No.: US 2013/0085105 A1
(43) Pub. Date: Apr. 4, 2013

(54) Transdermal Administration Of Peptides

(71) Applicants: Patrick Bernard Deasy, Dublin (IE); Thomas Ciaran Loughman, Dublin (IE)

(72) Inventors: Patrick Bernard Deasy, Dublin (IE); Thomas Ciaran Loughman, Dublin (IE)

(73) Assignees: The Provost, Fellows and Scholars of the College of Holy Trinity and Undivided Trinity of Queen Eli, Dublin (IE); Ipsen Manufacturing Ireland Limited, Blanchardstown, Dublin (IE)

(21) Appl. No.: 13/637,244

(22) PCT Filed: Mar. 23, 2011
(86) PCT No.: PCT/IE2011/000019

§ 371 Date: Dec. 11, 2012

(30) Foreign Application Priority Data
Mar. 25, 2010 (IE) 2010/0174

(51) Int. Cl. C07K 7/64

(52) U.S. Cl. CPC C07K 7/64
USPC 514/11.1; 530/311

(57) ABSTRACT

The present invention relates to a method of increasing the bioavailability of a peptide. The method includes altering the lipophilicity of a peptide by the creation of fatty acid peptide salts of the peptide. The fatty acid peptide salts exhibit increased transdermal and transmucosal permeability.

### FIELD OF THE INVENTION

This invention is directed to methods of increasing the permeability of peptides for penetration across dermal barriers.

### BACKGROUND OF THE INVENTION

Oral administration is one regimen commonly used for drug delivery because it is a relatively simple method and aids in maintaining patient compliance for the duration of the drug therapy. However, it is well known that many drugs taken orally are destroyed after passage through the liver; exposure to metabolic processes is particularly destructive for peptide or protein type drugs.

Transdermal drug delivery is an attractive alternative to more traditional drug delivery routes as a means of providing convenient and reliable systemic therapy for acute and chronic conditions. When compared to more conventional systems, transdermal delivery presents the following advantages (Kanikkannan, N., et al., Curr. Med. Chem., 7:593-608, 2000):
Avoidance of hepatic first pass metabolism and inactivation;
The ability to discontinue administration by easy removal of the system;
Continuous administration via the transdermal route at a controlled rate should eliminate the plasma troughs and peaks associated with conventional oral dosage forms and thus reduce the incidence of side effects;
The ability to modify the properties of the biological dermal barrier to absorption by using flux enhancers;
Increased patient compliance resulting from a relatively low level of associated pain;
The ability to avoid a changing physiological environment and chemical or metabolic degradation affecting drug absorption such as changing gastrointestinal pH or luminal microflora involvement in the gut;
A relatively large area of application is available in comparison with the buccal or nasal cavity; and
The transdermal route allows the administration of drugs with a narrow therapeutic index with a greater margin of safety.

A large number of variables affect the success of transdermal delivery and, as such, a variety of parameters affecting and/or modulating the drug, the carrier vehicle and the barrier have been investigated (see for example Malik et al., Curr. Drug Del., 4:141-151, 2007; Benson et al., J. Pharm. Science., 97:3591-3610, 2008). Transdermal drug delivery via the application of electrical current to the skin, e.g., iontophoresis or electroporation, is one means of introducing compounds to the body via movement into and across the skin (Hirvonen, J., et al., Nat. Biotech., 14:1710-1713, 1996; see also Nair et al., Pharm. Res., 47:563-569, 2003; Schuetz et al., European J. Pharm. Sci., 26:429-437, 2005; Schuetz et al., Expert Opin. Drug Deliv., 2:533-548, 2005). The disadvantages of electrical current techniques are considerable however, and include irritation, mild skin burns, limited drug flux, cost and issues with patient compliance (Guy, J. Pharm. Pharmacol., 50:371-374, 1998). Analogs of leutenizing hormone releasing hormone (LHRH) are one example of peptide drugs that have been assessed as potential candidates for iontophoretic delivery. The native peptide retains its immunological and biological activity under a constant current density of 0.2 mA/cm2 (Heit, M. C., et al., J. Pharm. Sci., 82:240-243, 1993) and significant drug depots in the skin underlying the electrode have been identified. However, the success with LHRH analogs has been elusive. While no formal relationship between peptide sequence, structure and iontophoretic efficiency has been established, it is clear that the lipophilic LHRH analogues nafarelin and leuprolide exhibit down-regulation of their own transdermal transport during iontophoresis (Hirvonen, J., et al., Nat. Biotech., 14:1710-1713, 1996). These lipophilic, cationic peptides may become anchored in the transport path, neutralising the original charge of the skin and completely altering its permselective properties. Thus, incorporation of bulky, lipophilic residues alongside cationic residues in the peptide sequence may lead to anomalous iontophoretic behaviour.

Lau et al. (Pharm. Res., 11:1742-1746, 1994) investigated iontophoresis of the somatostatin analogue octreotide in rabbits. An apparent bioavailability of approximately 8% and a therapeutic plasma concentration greater than 1 ng/ml throughout the iontophoretic episode was achieved. Therapeutic plasma concentrations have also been obtained for parathyroid hormone (PTH) via iontophoresis (Boericke, K., et al., Proc. Int. Symp. Contr. Rel. Bioact. Mat., 23:200-201, 1996.).

Hou et al. (Exp. Dermatol., 16:999-1006, 2007) and Lopes et al. (Eur. J. Pharm. Biopharm., 68:441-445, 2008) examined the use of protein transduction domains as a means of delivering peptides into animal cells and penetrating skin tissues. Low frequency ultrasound is yet another non-invasive method by which drugs can be delivered to a patient (see for example Smith, N. B., Int. J. Nanomedicin, 2:585-594, 2007). Creation of micropores in the skin (Garg et al., Clin. Vaccine Immunol., 14:926-928, 2007) was used to immunize mice via delivery of a virus protein; the bacterial pore-forming protein magainin was used to deliver flourescine through the skin (Kim, et al., J. Control Release, 122:375-383, 2007). Vesicle based transdermal delivery systems have also been developed (Benson, H. A., Expert Opin. Drug Deliv., 3:727-737, 2006) such as transfersomes. Microneedles and skin abrasion (Brown et al., Drug Delivery, 13:175-187, 2006) have also been proposed as a means of transversing the transdermal barrier for the delivery of drugs. Many researchers have used flux enhancers such as dimethylsulfoxide and related aprotic solvents dimethylformamide and dimethylacetamide, fatty alcohols, fatty acids, terpenes, propylene glycol and sodium lauryl sulfate to aid in permeation of the skin barrier and passage of desired compounds through the skin.

Due to the excellent barrier properties of the skin and the finding that the efficiency and compatibility of flux enhancers vary considerably, only a limited number of drugs have been successfully formulated to penetrate the skin in sufficient amounts to be of therapeutic use.

Scopolamine is an anti-muscarinic, used to prevent nausea and vomiting associated with motion, chemotherapy, surgery and opioid withdrawal symptoms. Oral and parenteral forms of the drug can cause dry mouth, drowsiness, confusion and blurred vision. The development of a transdermal scopolamine patch has been effective in prolonging the effective therapeutic time and minimizing any adverse side effects (Lin, Y. C., Paediatr. Anaesth., 11:626-627, 2001).

1,2,3-propanetriol trinitrate, commonly known as nitroglycerin, is indicated for the prophylaxis of angina pectoris and in the treatment of heart failure. Transdermal delivery of nitroglycerin offers a solution to the problem of its short therapeutic effect, a property common amongst nitrates when given orally, due to extensive first-pass metabolism. Several transdermal nitroglycerin formulations are commercially available including Deponit® (Schwarz Pharma), Nitro-Dur® (Key Pharmaceuticals) and Transderm-Nitro® (Novartis).

Transdermal estradiol, for use in hormone replacement therapy, offers a number of advantages over more traditional oral routes of delivery. Orally delivered estradiol is extensively metabolised in the gastro-intestinal tract to form a less active compound, estrone. It is also subject to first-pass metabolism which further reduces bioavailability. Oral delivery thus requires high initial doses to achieve therapeutic plasma levels. Since the transdermal route avoids biological degradation via the hepatic first-pass effect, it is possible to substantially reduce the dosage delivered compared to the oral route (Munoz, A., Maturitas, 33:S39-S47, 1999). Estraderm® (Novartis), Climara® (Schering) and Oesclim® (Groupe Fournier) are a few examples of transdermally delivered estradiol.

Nicotine replacement therapy has proved to be an effective approach towards combating the effects of smoking cessation and transdermal patches are widely used for nicotine substitution. Commercially available nicotine patches include Habitrol® (Novartis) and Nicoderm® (Glaxo SmithKline).

Clonidine is an α-adrenoreceptor imidazoline derivative agonist used in the treatment of hypertension. Transdermal delivery of the drug reduces adverse effects, such as dry mouth and drowsiness, and can provide steady-state concentrations for up to one week. Catapres-TTS® (Boehringer Ingelheim) is a membrane permeation-controlled device currently available

Duragesic® (Janssen Pharmaceutics) is a membrane permeation-controlled transdermal delivery path providing continuous systemic delivery of fentanil, a potent opioid analgesic. Transdermal fentanil has been compared favourably with oral morphine in paediatric palliative care (Hunt, A., et al., Palliat. Med., 15:405-412, 2001) and in treating non-cancer pain (Dellemijn, P. L., Eur. J. Pain, 5:333-339, 2001).

Testosterone is the primary endogenous androgenic hormone responsible for the normal growth and development of the male sex organs and for maintenance of secondary sex characteristics. Testosterone replacement therapy is currently used in cases of hypogonadism (i.e. the absence of testosterone), but may in future also be used as a supplement to treat patients with decreased testosterone levels (Fortunato, L., et al., 3M Delivery, 15:6-7, 2000). Testoderm® (Alza Corporation) is one example of a transdermally delivered testosterone treatment.

As is demonstrated by the examples cited above, transdermal delivery methods have proven most useful for the transport of small molecules rather than peptide-based drugs. Early work carried out by Weber et al. (Surgery, 102:974-981, 1987) investigated the transdermal delivery of the somatostatin analogue octreotide. Topical administration of octreotide along with 1% of the penetration enhancer N-decylmethylsulfoxide to mice resulted in clinically significant plasma levels of peptide (>8 ng/ml) within 2 hours of administration. The activity of the transferred peptide was confirmed by the accompanied dramatic hypoglycaemia, an indication of somatostatin action. Complementary in vitro analyses allowed them to conclude that the peptide would cross both human and mouse skin with a clinically significant flux when administered topically with N-decylmethylsulfoxide. Chemical flux enhancers such as N-decylmethylsulfoxide can be quite harsh, however, as they often modify peptides and proteins in the skin bilayer and cause ruptures in the stratum corneum.

Due to relatively high potencies with low doses required for therapeutic effects, peptide drugs may seem to be excellent candidates for transdermal therapeutic products. As peptides are generally large hydrophilic molecules often with limited stability resulting in minimal transdermal bioavailability, however, no commercial product has been developed to-date. Hydrophilicity and stability issues also make it extremely difficult to successfully deliver peptides via any other routes across biological membranes such as gastrointestinal, buccal, rectal, nasal or pulmonary routes. Parenteral remains the main route for peptide administration, but this can lead to poor patient compliance. The advantages associated with transdermal delivery (avoidance of first-pass metabolism, avoidance of significant drug degradation, increased patient compliance, etc.) would be highly desirable for peptide and protein drugs.

There remains in the art a need for safe, effective and simple transdermal administration of physiologically active peptide drug agents.

### SUMMARY OF THE INVENTION

In one embodiment, the invention provides a method of altering the bioavailability of a peptide. In one aspect, the bioavailability of the peptide is altered by modifying the lipophilicity of the peptide. In yet a further aspect, the lipophilicity of the peptide is increased by preparing a fatty acid salt of the peptide. In a further aspect, the fatty acid peptide salt exhibits increased transdermal permeability. In yet another aspect, the fatty acid peptide salt exhibits increased transmucosal permeability.

In a second embodiment, the invention provides a composition for transdermal delivery comprising a fatty acid salt of a peptide.

In a third embodiment, the invention provides a transdermal administration device comprising a fatty acid salt of a peptide.

In one aspect, the fatty acid salts useful in the practice of the invention include any fatty acid, such as but not limited to, saturated or unsaturated fatty acids. Exemplary fatty acids include, but are not limited to, octanoic acid, nonanoic acid, decanoic acid, dodecanoic acid, tetradecanoic acid, cis-9-octadecanoic acid, cis,cis-9,12-octadecanoic acid and the like. In one aspect, the fatty acid is octanoic acid.

The number and type of peptides useful in the practice of any of the embodiments of this invention are numerous, and include, but are not limited to, peptides, peptide analogs, proteins, protein analogs, peptide hormones, peptide hormone analogs, enzymes and the like. Exemplary peptide analogs include, but are not limited to analogs, agonists and antagonists of bombesin, leutinizing hormone releasing hormone, somatostatin, glugacon-like peptide-1, glucose-dependent insulinotropic polypeptide, melanocortins, melanocyte stimulating hormone, farnesyl transferase inhibitors, growth hormone, growth hormone releasing factor/hormone, growth hormone secretagogues, parathyroid hormone, parathyroid related hormone, pituitary adenylate cyclase activating polypeptide, urotensin II, ghrelin, peptide YY, Mullerian inhibiting substance, adropin, parathyroid related peptide, neuropeptide Y, dopastatin, exendin, insulin-like growth factor-1, adrenocorticotropic hormone, peptides affecting apoptosis, agents useful to reduce the proliferation of hemopoietic cells, cholecystokinins, chemokines, opioid peptides, cytotoxic peptide conjugates and the like.

In another embodiment, the peptides, peptide analogs, proteins, protein analogs, peptide hormones, peptide hormone analogs, enzymes and the like. Exemplary peptide analogs include, but are not limited to analogs, agonists and antagonists of bombesin, leutinizing hormone releasing hormone, somatostatin, glugacon-like peptide-1, glucose-dependent insulinotropic polypeptide, melanocortins, melanocyte stimulating hormone, farnesyl transferase inhibitors, growth hormone, growth hormone releasing factor/hormone, growth hormone secretagogues, parathyroid hormone, parathyroid related hormone, pituitary adenylate cyclase activating polypeptide, urotensin II, ghrelin, peptide YY, Mullerian inhibiting substance, adropin, parathyroid related peptide, neuropeptide Y, dopastatin, exendin, insulin-like growth factor-1, adrenocorticotropic hormone, peptides affecting apoptosis, agents useful to reduce the proliferation of hemopoietic cells, cholecystokinins, chemokines, opioid peptides, and the like are prepared to contain cytotoxic moieties such as camptothecin or other toxic agents.

In one aspect, any of the embodiments of this invention are practiced using fatty acid salts of somatostatin agonists or antagonists. In a further aspect, the somatostatin agonists or antagonists are selective for the somatostatin type-1 receptor. Exemplary SSTR-1 receptor agonists include, but are not limited to fatty acid salts of:

Taeg-c(D-Cys-3-Pal-D-Trp-Lys-D-Cys)-Thr(Bzl)-Tyr-NH₂ and

Caeg-c(D-Cys-3-Pal-D-Trp-Lys-D-Cys)-Thr(Bzl)-Tyr-NH₂.

In a further aspect, the somatostatin agonists or antagonists are selective for the somatostatin type-2 receptor. Exemplary type-2 somatostatin agonists include, but are not limited to fatty acid salts of:
Lanreotide D-2-Nal-c(Cys-Tyr-D-Trp-Lys-Val-Cys)-Thr-NH₂;
Octreotide D-Phe-c(Cys-Phe-D-Trp-Lys-Thr-Cys)-Thr-ol;
[4-(2-hydroxyethyl)]-1-piperazinylacetyl-D-Phe-c(Cys-Tyr-D-Trp-Lys-Abu-Cys)-Thr-NH₂;
c(Tic-Tyr-D-Trp-Lys-Abu-Phe);
4-(2-Hydroxyethyl)-1-piperazine-2-ethanesulfonyl-D-Phe-c(Cys-Tyr-D-Trp-Lys-Abu-Cys)-Thr-NH₂;
[4-(2-hydroxyethyl)]-1-piperazinylacetyl-D-Phe-c(Cys-Tyr-D-Trp-Lys-Abu-Cys)-Thr-NH₂;
D-6-propyl-8beta-ergolinylmethyl-thioacetyl-D-Lys(D-6-propyl-8beta-ergolinyl-methylthioacetyl)-c(Cys-Tyr-D-Trp-Lys-Abu-Cys)-Thr-NH₂;
D-Cpa-c(Cys-Tyr-D-Trp-Lys-Val-Cys)-Thr-NH₂;
D-Phe-Cpa-Tyr-D-Trp-Lys-Val-Phe-Thr-NH₂;
D-Phe-Cpa-Tyr-D-Trp-Lys-Thr-Phe-Thr-NH₂; and
Ac-c(Cys-Lys-Asn-Cpa-Phe-D-Trp-Lys-Thr-Phe-Thr-Ser-D-Cys)-NH₂;

Exemplary fatty acid salts of type-2 somatostatin agonists include, but are not limited to, di-oleate salts of D-2-Nal-c(Cys-Tyr-D-Trp-Lys-Val-Cys)-Thr-NH₂, D-Phe-c(Cys-Phe-D-Trp-Lys-Thr-Cys)-Thr-ol and [4-(2-hydroxyethyl)]-1-piperazinylacetyl-D-Phe-c(Cys-Tyr-D-Trp-Lys-Abu-Cys)-Thr-NH₂.

Exemplary type-2 somatostatin antagonists include, but are not limited to fatty acid salts of:
Cpa-c(D-Cys-3-Pal-D-Trp-Lys-Val-Cys)-Cpa-NH₂;
4FPhe-c(D-Cys-3-Pal-D-Trp-Lys-Tle-Cys)-2-Nal-NH₂;
Cpa-c(D-Cys-3 Pal-D-Trp-Lys-Thr-Cys)-2-Nal-NH₂; and
Cpa-c(D-Cys-4-Pal-D-Trp-Lys-Thr-Cys)-2-Nal-NH₂.

In a further aspect, the somatostatin agonists or antagonists are selective for the somatostatin type-3 receptor. In a further aspect, the somatostatin agonists or antagonists are selective for the somatostatin type-4 receptor.

In a further aspect, the somatostatin agonists or antagonists are selective for the somatostatin type-5 receptor. Exemplary type-5 somatostatin agonists include, but are not limited to fatty acid salts of:
D-Phe-Phe-Trp-D-Trp-Lys-Thr-Phe-Thr-NH₂ and
c(Cys-Phe-Phe-D-Trp-Lys-Thr-Phe-Cys)-NH₂.

In yet a further aspect, the somatostatin agonists or antagonists are selective for a combination of at least two of somatostatin type-1, type-2, type-3, type-4 or type-5 receptors or any combination thereof.

In one aspect, any of the embodiments of this invention are practiced using fatty acid salts of agents useful to reduce the proliferation of hemopoietic cells during chemotherapy or radiotherapy, such as but not limited to, AcSDKP(CH₃—CO-Ser-Asp-Lys-Pro-OH) (SEQ ID NO:1), to administer angiotensin-converting enzyme (ACE) inhibitor agonists, and to administer a hemopoiesis growth factor.

In one aspect, any of the embodiments of this invention are practiced using fatty acid salts of bombesin agonists or antagonists. Exemplary bombesin analogs include, but are not limited to:

(SEQ ID NO: 2)
H-pGlu-Gln-Ser-Leu-Gly-Asn-Gln-Trp-Ala-Arg-Gly-His-Phe-Met-NH₂;
(SEQ ID NO: 3)
Gly-Asn-Gln-Trp-Ala-Arg-Gly-His-Phe-Met-NH₂;
and
H-D-F5-Phe-Gln-Trp-Ala-Val-D-Ala-His-Leu-O-CH₃.

In one aspect, any of the embodiments of this invention are practiced using fatty acid salts of cholecystokinin antagonists.

In one aspect, any of the embodiments of this invention are practiced using fatty acid salts of chemokine analogs. Exemplary chemokine analogs include, but are not limited to:

(SEQ ID NO: 4)
Ac-c(Cys-Leu-Asp-Pro-Lys-Gln-Lys-Trp-Ile-Gln-Cys)-
NH₂;
(SEQ ID NO: 5)
c(Cys-Trp-Ile-Gln-Cys)-NH₂;
or
(SEQ ID NO: 6)
c(Gly-Lys-Trp-Ile-Gln-Glu)-NH₂.

In one aspect, any of the embodiments of this invention are practiced using fatty acid salts of opioid peptides.

In one aspect, any of the embodiments of this invention are practiced using fatty acid salts of ghrelin/growth hormone releasing hormone agonist or antagonist peptides. Exemplary ghrelin/growth hormone releasing hormone analogs include, but are not limited to, (Aib², Glu³(NH-hexyl))hGhrelin(1-28)-NH₂ (SEQ ID NO:7) and H-Inp-D-Bal-D-Trp-Phe-Apc-NH₂.

In one aspect, any of the embodiments of this invention are practiced using fatty acid salts of glugacon-like peptide-1 (GLP-1) agonist or antagonist peptides. Exemplary GLP-1 agonists include, but are not limited to, (Aib^{8,35})hGLP-1(7-36)NH₂ (SEQ ID NO:8), (Ser⁸, Aib³⁵) hGLP-1 (7-36) NH₂ (SEQ ID NO:9) and [Aib^{8,35,}Arg³⁴]hGLP1(7-36)-NH₂(SEQ ID NO:10).

In one aspect, any of the embodiments of this invention are practiced using fatty acid salts of luteinizing hormone releasing hormone (LHRH) agonist or antagonist peptides. Exemplary LHRH agonists include, but are not limited to, pGlu-His-Trp-Ser-Tyr-D-Trp-Leu-Arg-Pro-Gly-NH₂.

In one aspect, any of the embodiments of this invention are practiced using fatty acid salts of melanocortin receptor agonist or antagonist peptides. Exemplary melanocortin receptor-4 agonist peptides include, but are not limited to, hydantoin(Arg-Gly)-c(Cys-Glu-His-D-Phe-Arg-Trp-Cys)-NH₂.

In one aspect, any of the embodiments of this invention are practiced using fatty acid salts of pituitary adenylate cyclase activating polypeptide (PACAP) analogs.

In one aspect, any of the embodiments of this invention are practiced using fatty acid salts of parathyroid hormone or parathyroid hormone releasing hormone peptide agonists. Exemplary parathyroid hormone releasing hormone peptide agonists include, but are not limited to, Glu^{22,25,}Leu^{23,28,31,}Aib^{29,}Lys^{26,30}]hPTHrP(1-34)-NH₂ (SEQ ID NO:11).

In one aspect, any of the embodiments of this invention are practiced using fatty acid salts of peptide Y or neuropeptide Y agonists. Exemplary peptide Y or neuropeptide Y agonists include, but are not limited to, [camptothecin-rvGly-Suc-Tyr¹, Nle¹⁷, Pro³⁴]hNPY(1-36)-NH₂ (SEQ ID NO:12), [camptothecin-rvD/LAsp-Suc-Tyr¹, Nle¹⁷, 4Hyp³⁴]hNPY(1-36)-NH₂ and [camptothecin-rvD/L-Asp-Suc-Tyre, Nle¹⁷, A6c³¹, 4Hyp³⁴]hNPY(1-36)-NH₂.

In one aspect, any of the embodiments of this invention are practiced using fatty acid salts of ADROPIN c(Cys-His-Ser-Arg-Ser-Ala-Asp-Val-Asp-Ser-Leu-Ser-Glu-Ser-Ser-Pro-Asn-Ser-Ser-Pro-Gly-Pro-Cys)-Pro-Glu-Lys-Ala-Pro-Pro-Pro-Gln-Lys-Pro-Ser-His-Glu-Gly-Ser-Tyr-Leu-Leu-Gln-Pro (SEQ ID NO:13) or analogs thereof.

In one aspect, any of the embodiments of this invention are practiced using fatty acid salts of urotensin II agonists or antagonists. Exemplary urotensin II agonists include, but are not limited to, Asp-c[Cys-Phe-Trp-Lys-Tyr-Cys]-Val-OH (SEQ ID NO:14). Exemplary urotensin II antagonists include, but are not limited to, Cpa-c[D-Cys-Pal-D-Trp-Lys-Val-Cys]-Cpa-NH₂ and Cpa-c[D-Cys-Phe-Trp-Lys-Thr-Cys]-Val-NH₂.

In one aspect, any of the embodiments of this invention are practiced using fatty acid salts of glucose-dependent insulinotropic polypeptide (GIP) analogs. Exemplary glucose-dependent insulinotropic polypeptide analogs include, but are not limited to,

(D-Ala², A5c¹¹, His⁴³)hGIP(1-43)-OH;
(D-Ala², A5c^{11,41})hGIP(1-42)-OH;
(SEQ ID NO: 15)
(Gly², A5c¹¹, Nle¹⁴, His⁴³)hGIP(1-43)-OH;
(D-Ala², A5c¹¹, Nle¹⁴, His⁴³)hGIP(1-43)-OH;
(SEQ ID NO: 16)
(A5c^{11,41})hGIP(1-42)-OH;
(SEQ ID NO: 17)
(A5c^{11,40})hGIP(1-42)-OH;
(SEQ ID NO: 18)
(A5c¹¹, His⁴³)hGIP(1-43)-OH;
(SEQ ID NO: 19)
(A5c¹¹, Asn⁴³)hGIP(1-43)-OH;
(SEQ ID NO: 20)
(Aib¹³, Asp⁴³)hGIP(1-43)-NH₂;
(SEQ ID NO: 21)
(Aib¹³, Nle¹⁴, A5c⁴⁰)hGIP(1-42)-OH;
(SEQ ID NO: 22)
(Aib¹³, A5c⁴⁰)hGIP(1-42)-OH;
(SEQ ID NO: 23)
(A5c^{11,14}, His⁴³)hGIP(1-43)-OH;
(SEQ ID NO: 24)
(3Cl-Tyr¹, Aib², A5c¹¹, Nle¹⁴)hGIP(1-42)-OH;
(SEQ ID NO: 25)
(3MeO-Tyr¹, Aib², A5c^{11,41})hGIP(1-42)-OH;
SEQ ID NO: 26
(Aib², A5c¹¹, Nle¹⁴)hGIP(1-42)-OH;
SEQ ID NO: 27
[A5c¹¹, Lys⁴³(N—C(O)—(CH₂)₁₄—CH₃)]hGIP(1-43)-OH;
[3Cl-Tyr¹, D-Ala², A5c¹¹, Nle¹⁴, Lys⁴³(N—C(O)—(CH₂)₁₀—CH₃)]hGIP(1-43)-OH;
SEQ ID NO: 28
[3Cl-Tyr¹, Aib², A5c¹¹, Nle¹⁴, Cys⁴³(succinimide-N-30K PEG)]hGIP(1-43)-NH₂;
SEQ ID NO: 29
[3Cl-Tyr¹, Aib², A5c¹¹, Nle¹⁴, Cys⁴³(succinimide-N—(CH₂)₂—C(O)NH—(CH₂)₃—O—CH₂-
CH(20K PEG)-CH₂-20K PEG)]hGIP(1-43)-NH₂;
SEQ ID NO: 30
(Aib², A6c⁷)hGIP(1-30)-NH₂,
SEQ ID NO: 31
(Aib^{2,13}, Nle¹⁴)hGIP(1-42)-OH;
(D-Ala², A5c^{11,14})hGIP(1-30)-NH₂;
SEQ ID NO: 32
[A5c¹¹, Lys⁴³(N—C(O)—(CH₂)₁₀—CH₃)]hGIP(1-43)-OH;
SEQ ID NO: 33
(A5c^{11,41}, Cys⁴³)hGIP(1-43)-NH₂;
SEQ ID NO: 34
(hTyr¹, Aib², A5c¹¹, His⁴³)hGIP(1-43)-OH;
[3Cl-Tyr¹, D-Ala², A5c^{11,14}, Lys⁴³(N—C(O)—(CH₂)₁₀—CH₃)]hGIP(1-43)-OH;
[3Cl-Tyr¹, D-Ala², A5c¹¹, Nle¹⁴, Cys⁴³(succinimide)]hGIP(1-43)-OH;
SEQ ID NO: 35
(Ac-A6c⁷, Gln⁴³)hGIP(7-43)-OH;
SEQ ID NO: 36
(4Hppa², Aib¹³)hGIP(2-42)-OH;
SEQ ID NO: 37
(3Cl-Tyr¹, Aib², A5c¹¹, Nle¹⁴, Cys⁴³)hGIP(1-43)-OH;
SEQ ID NO: 38
[3Cl-Tyr¹, Aib², A5c¹¹, Nle¹⁴, Cys⁴³(succinimide-N-20K PEG)]hGIP(1-43)-NH₂;
SEQ ID NO: 41
[A5c^{11,41}, Cys⁴³(succinimide-N-30K PEG)]hGIP(1-43)-NH₂.

In one aspect, any of the embodiments of this invention are practiced using fatty acid salts of insulin-like growth factor-1 (IGF-1) analogs. Exemplary insulin-like growth factor-1 (IGF-1) analogs include, but are not limited to, those having a natural or non-natural amino acid substituted for Met at position 59.

In one aspect, any of the embodiments of this invention are practiced using fatty acid salts of peptides that promote and/or control apoptosis. Exemplary apoptotic control genes include peptides containing a BH-3 domain such as, but not limited to:

(SEQ ID NO: 39)
Ac-Leu-Ser-Glu-Cys-Leu-Lys-Arg-Ile-Gly-Asp-Glu-Leu-Asp-Ser-Asn-
NH₂
and
(SEQ ID NO: 40)
Ac-Leu-Ser-Glu-Ser-Leu-Lys-Arg-Ile-Gly-Asp-Glu-Leu-Asp-Ser-Asn-
NH₂.

In one aspect, any of the embodiments of this invention are practiced using fatty acid salts of peptides that act as biological receptor ligands joined with a cytotoxic moiety. Exemplary peptide-cytotoxic conjugates include, but are not limited to, conjugates of anthracycline cytotoxic agents and fatty acid salts of peptide hormones such as LHRH, bombesin or somatostatin such as
Camptothecin-Gly-glutaryl-AEPA-Lys-D-Tyr-D-Tyr-c(Cys-Tyr-D-Trp-Lys-Abu-Cys)-Thr-NH₂;
Camptothecin-Gly-glutaryl-Lys-D-Tyr-DTyr-c(Cys-Tyr-D-Trp-Lys-Abu-Cys)-Thr-NH₂;
Camptothecin-20-glycinyl-succinoyl-D-Phe-c(Cys-Tyr-D-Trp-Lys-Abu-Cys)-Thr-NH₂;
Doxorubicin-Gly-Suc-(Doc)₄-Aepa-D-Phe-c(Cys-Tyr-D-Trp-Lys-Abu-Cys)-Thr-NH₂;
Camptothecin-Gly-Glut-(Doc)₄-Lys-D-Tyr-DTyr-cyclo(Cys-Tyr-D-Trp-Lys-Abu-Cys)-Thr-NH₂;
Camptothecin-CONH—(CH₂)₂—S—S—(CH₂)—CO-(Doc)₄-Lys-D-Tyr-DTyr-c(Cys-Tyr-D-Trp-Lys-Abu-Cys)Thr-NH₂;
Camptothecin-rvD-Asp-suc-(Doc)₄-Aepa-D-Phe-c(Cys-Tyr-D-Trp-Lys-Abu-Cys)-Thr-NH₂;
Camptothecin-rvCha-suc-(Doc)₄-Aepa-D-Phe-c(Cys-Tyr-D-Trp-Lys-Abu-Cys)-Thr-NH₂;
Camptothecin-rvAbu-suc-(Doc)₄-Aepa-D-Phe-c(Cys-Tyr-D-Trp-Lys-Abu-Cys)-Thr-NH₂;
Camptothecin-rvVal-suc-(Doc)₄-Aepa-D-Phe-c(Cys-Tyr-D-Trp-Lys-Abu-Cys)-Thr-NH₂;
Camptothecin-C(O)—N((CH₂)₂NHCH₃)-β-Ala-(Doc)₄-Aepa-D-Phe-c(Cys-Tyr-D-Trp-Lys-Abu-Cys)-Thr-NH₂;
CamptothecinSN38-rvGly-Suc-(Doc)₄-Aepa-D-Phe-c(Cys-Tyr-D-Trp-Lys-Abu-Cys)-Thr-NH₂;
Camptothecin-rvD-Asp-Suc-(Peg₃)₃-Aepa-D-Phe-c(Cys-Tyr-D-Trp-Lys-Abu-Cys)-Thr-NH₂; and
Paclitaxel-glutaryl-D-Phe-c(Cys-Phe-D-Trp-Lys-Thr-Cys)-Thr-ol.

In one aspect, any of the embodiments of this invention are practiced using fatty acid salts of peptides in combination with flux enhancers such as dimethylsulfoxide (DMSO) and related aprotic solvents dimethylformamide and dimethylacetamide, fatty alcohols such as decanol and dodecanol, fatty acids such as oleic acid, terpenes such as 1,8-cineole, propylene glycol, sodium lauryl sulfate and the like.

### BRIEF DESCRIPTION OF THE DRAWINGS

FIG. 1 Diffusion Cell for transdermal permeation studies. 1) transdermal skin system; 2) expansion port; 3) donor compartment; 4) feceiver compartment; 5) sampling port; 6) stirring bar; 7) water jacket.

FIG. 2 Panel 1) Transdermal flux profiles obtained for DMSO solutions of Compound A (D-2-Nal-c(Cys-Tyr-D-Trp-Lys-Val-Cys)-Thr-NH₂) and Compound B ([4-(2-hydroxyethyl)]-1-piperazinylacetyl-D-Phe-c(Cys-Tyr-D-Trp-Lys-Abu-Cys)-Thr-NH₂) oleate in vitro across hairless mouse skin (n=5); Panel 2) Transdermal flux profiles obtained for DMSO solutions of various Compound A salts and Compound B acetate in vitro across hairless mouse skin (n=3-5).

FIG. 3 Transdermal flux profiles obtained for Compound A oleate at different concentrations in DMSO in vitro across hairless mouse skin (n=5).

FIG. 4 Panel 1) In vitro transdermal flux profiles obtained for Compound A oleate in various solvents across hairless mouse skin (n=5). Panel 2) In vitro transdermal flux profiles obtained for Compound B oleate in various solvents across hairless mouse skin (n=5).

FIG. 5 In vitro transdermal flux profile obtained for Compound A acetate in DMSO+1% (v/v) oleic acid across hairless mouse skin (n=5).

FIG. 6 In vitro transdermal flux profile obtained for Compound B oleate in 55/45 (v/v) EtOH/water with and without 0.5% (v/v) SLS across hairless mouse skin (n=4).

FIG. 7 In vitro transdermal flux profile obtained for Compound A and Compound B oleate in 55/45 (v/v) EtOH/water with and without 5% 1,8-Cineole across (Panel 1) hairless mouse skin or (Panel 2) a comparison of flux profiles with 5% cineole across hairless mouse and pig skin (n=4-5).

FIG. 8 In vitro transdermal flux profiles across pig epidermis obtained for Compound B oleate in various donor vehicles (n=3-5).

FIG. 9 In vitro transdermal flux profiles across microporous polypropylene and pig epidermis for 5 mg/ml Compound B oleate in 55/40/5 (v/v/v) EtOH/H₂O/cineole with 5% (w/v) HPC (n=5).

FIG. 10 Schematic of the patch assembled for use in in vivo studies. 1) metallized polyester laminate; 2) foam ring; 3) adhesive rim; 4) drug reservoir.

FIG. 11 Panel 1) Individual and average (n=4) Compound B levels in pig plasma following application of patch A containing 1 ml of a solution of 5 mg/ml Compound B or Compound A as oleate salt in 95/5 (v/v) ethanol/cineole gelled with 2.5% (w/v) HPC. The X-axis crosses the Y-axis at the assay limit of detection (21 pg/ml); Panel 2) Average Compound B (n=3) and Compound A (n=2) levels in pig plasma following application of patch B containing 5 mg/ml Compound B or Compound A as oleate salt in 95/5 (v/v) ethanol/cineole gelled with 2.5% (w/v) HPC. The X-axis crosses the Y-axis at the assay limit of detection (21 pg/ml).

FIG. 12 Average levels of Compound A in pig plasma following application of various ointments. The X-axis crosses the Y-axis at the assay limit of detection (21 pg/ml; n=2). Panel 1) Compound B/wool alcohols ointment/ethanol/cineole. Panel 2) Compound B/Plastibase/ethanol/cineole ointment. Panel 3) Compound B/emulsifying ointment/ethanol/cineole. Panel 4) Compound B/Macrogol ointment/ethanol/cineole.

### DETAILED DESCRIPTION OF THE INVENTION

The human skin consists of a stratified, a vascular cellular epidermis and an underlying dermis of connective tissue. Epidermal cells travel from the proliferative layer of the basal cells and change from being metabolically active cells to dead, dense cells. Below the epidermis lies the dermis or corium, consisting of a matrix of connective tissue made up of fibrous proteins. Within this network there exist blood vessels, nerves and lymphatic vessels. There are also direct openings from the dermis to the skin surface in the form of sebaceous glands, sweat (eccrine) glands and hairs. As the dermis also has a rich blood supply, it is possible for topically applied materials to penetrate directly through these openings into systemic circulation. Below the dermis lies a layer of subcutaneous fat; which acts as a thermal barrier and a mechanical cushion, as well as a site of synthesis and a depot of readily available high-energy chemicals (see Barry, B. W., Dermatological formulations: percutaneous absorption in Drugs and the Pharmaceutical Sciences 18, Marcel Dekker Inc., New York, 1983).

With regard to the percutaneous absorption of drug molecules, however, it is the outermost layer of the skin, the stratum corneum, which is the dominant factor in controlling the penetration of molecules applied to the skin. Generally, this region consists of 10-15 layers of flattened, keratinized, metabolically inactive cells that are stacked in highly organized units of vertical columns (Christophers, E., J. Invest. Dermatol., 56:165-169, 1971). The stratum corneum (SC) is virtually devoid of phospholipids and is rich in ceramides, free sterols, and free fatty acids, with smaller quantities of glycolipids, sterol esters, triglycerides, cholesterol sulphate and hydrocarbons (Elias, P. M., The importance of epidermal lipids for the stratum corneum, in Topical Drug Delivery Formulations, Osborne, D. W. and Amann, A. H. (Eds.), Marcel Dekker Inc., New York, 1990).

The skin performs many functions including, but not limited to, the containment of tissue, protection from potentially harmful external stimuli, environmental sensing and regulation of body temperature and blood pressure. The protective function of the skin is based on the ability of the SC to act as a barrier to microorganisms, chemicals, radiation, temperature and electricity. Human skin acts as a barrier in two directions, controlling the loss of water and other body constituents from the body to the outside environment while preventing the entry of unwanted molecules from the outside environment into the body. It is possible, however, for chemicals to achieve systemic circulation via the shunt route of the appendages. Overall, intact skin provides an effective barrier to chemical permeation as the diffusional resistance of the SC is large for virtually all molecular species except gases.

When a molecule reaches the surface of the skin, it first encounters the sebum, bacteria and other exogenous materials which coat the skin surface. Three possible pathways are available for the drug to penetrate through the skin: 1) across the intact SC, 2) through the sweat glands or 3) via the hair follicles.

Under normal conditions, transport through either the sweat glands or hair follicles is thought to be insignificant, mainly due to the low surface area occupied by these appendages; the follicular area accounts for 0.1% and the sweat glands account for only 0.001% of the total skin surface area (Hadgraft, J., Int. J. Pharm., 224:1-18, 2001).

Transport across intact SC occurs via a transcellular and/or an intercellular route. Using a vapor fixation technique, Boddé et al. (J. Control. Rel., 15:227-236, 1991) provided evidence in support of the intercellular route as the main pathway for transdermal penetration with the transcellular pathway acting as a secondary passage route. Contribution of intercellular or transcellular pathways in the percutaneous absorption of drugs depends on the diffusivitiy of the drug in lipids and proteins, partitioning of the drug between these domains and the geometry of the SC (Moghimi, H. R., et al., Stratum corneum and barrier performance in Percutaneous Absorption, Bronaugh, R. L. and Maibach, H. I. (Eds.), Marcel Dekker Inc., New York, 1999). Taking the intercellular route as the dominant route, two obvious reasons for the relative impermeability of the skin become apparent 1) the tortuous route through the various skin layers and 2) the problem of repeated partition and diffusion across structured bilayers. This impermeability is a considerable problem in the delivery of drugs, particularly peptide drugs, both to and through the skin.

The release of a therapeutic peptide agent from a topically applied formulation and its transport into systemic circulation is a multi-step process which involves: (a) dissolution within and release from the formulation; (b) partitioning into the SC; (c) diffusion through the SC, principally via a lipidic intercellular pathway; (d) partitioning from the SC into the aqueous viable epidermis; (e) diffusion through the viable epidermis and into the upper dermis; and (f) uptake into the local capillary network and eventually systemic circulation (Kalia, Y. N., et al., Adv. Drug. Del. Rev., 48:159-172, 2001). Thus, the permeation process, whereby the molecule migrates from the vehicle through the skin and into the systemic circulation, is affected by three individual components and any interactions between these components: the skin, the drug and the vehicle.

Of the biological factors affecting the skin which impact transdermal drug delivery, age and condition, regional site, species differences, metabolism and circulatory effects are considered the most significant. For example, Behl et al. (Influence of age on percutaneous absorption of drug substances, in Percutaneous Absorption: Mechanisms-Methodology-Drug Delivery, Bronaugh, R. L. and Maibach, H. I. (Eds.), Marcel Dekker Inc., New York, 1985) demonstrated that aged skin, from both humans and hairless mice, was several times less permeable than young skin.

Skin condition also impacts permeability. While some agents (e.g. acids and alkalis) can promote permeation by effectively injuring barrier cells, disease is a much more common cause of alteration in skin condition. Wertz and Downing (Stratum corneum: Biological and biochemical considerations in Transdermal Drug Delivery, Hadgraft, J. and Guy, R. H. (Eds.), New York and Basel, 1988) noted that among diseases that alter the properties of the SC, the most notable are the ichthyoses and psoriasis. The condition of the SC can also be affected by essential fatty acid deficiencies whereby any insufficiency of linoleate in the diet produces scaly skin and transepidermal water loss rapidly increases. Another common disorder is dry skin, a condition which is almost universal in persons over the age of 65.

Feldmann and Maibach (J. Invest. Dermatol., 48:181-183, 1967) utilized the topical application of hydrocortisone to explore the potential variation in absorption depending on the site of topical application on the body. The scrotum was the highest absorbing skin site followed by the areas around the head and face, while the lowest absorption was found in the foot area. Thus, the site of application is clearly important when considering skin absorption in humans.

The skin can metabolize compounds before they enter the bloodstream. Enzymes in the SC, and probably in the sebum, are able to hydrolyze certain chemicals (for example, esters). Lipase, protease, phosphatase, sulphatase and glycosidase activities have been identified in the SC (Howes, D., et al., ATLA, 24:81-106, 1996). Additionally, the skin recognizes if normal barrier function has been impaired and rapidly restores itself by synthesizing lipids to replace any that have been extracted.

Knowledge of skin surface metabolism activity is important for risk assessment purposes and in terms of designing a transdermal drug and delivery combination. If the drug of interest undergoes biotransformation during its passage through the SC into metabolites which penetrate the skin to a greater extent than the parent compound, metabolism is obviously a critical determinant of percutaneous absorption. However, if metabolism occurs after the normal rate-limiting passage of the material across the SC into the pilosebaceous system or epidermis, the material is usually regarded as having penetrated the skin and as being systemically available.

Circulation also has an effect on percutaneous absorption in that an increased blood flow may reduce the time for which a penetrant remains in the dermis and thus raise the concentration gradient across the skin. Hydration of the skin is also an important factor in drug permeation as it has been shown that hydrating the SC increases the permeation of many molecules through the skin (Barry, B. W., Dermatological formulations: percutaneous absorption in Drugs and the Pharmaceutical Sciences 18, Marcel Dekker Inc., New York, 1983). The increase in permeation of most substances through the skin that accompanies hydration apparently results from an increase in diffusivity, primarily due to an alteration of the polar route (Lambert, W. J., et al., J. Pharm. Sci., 78:925-928, 1989).

A wide range of surrogate animal models are available for the study of skin and particularly for the study of skin permeability for various compounds, formulations and solutions (Howes, D., et al., ATLA, 24:81-106, 1996). The most common models include hairless mouse, hairless rat and domestic pig. Table 1 shows the differences in various skin layer thicknesses for human, hairless mouse and domestic pig skin.

TABLE 1

| | Thickness of Skin (μm) | | |
|---|---|---|---|
| Species | Full Thickness | Epidermis | Stratum Corneum |
| Human | 3,300 | 49.5 | 18.4 |
| Hairless Mouse | 1,230 | 15.0 | 3.8 |
| Domestic Pig | 1,600 | 50.0 | 10.0 |

Several in vitro studies have demonstrated that hairless mouse skin is more susceptible to chemical perturbations than human skin and that the use of chemical penetration enhancers can result in a far greater increase in flux across hairless mouse skin than across human skin (Simon, G. A., et al., Skin Pharmacol. Appl. Skin Physiol., 11:80-86, 1998). As shown by Lauer et al. (J. Pharm. Sci., 86:13-18, 1997), hairless rodent skin contains patulous cysts and enlarged, highly vascularized sebaceous glands that are hypothesized to enhance polar transdermal pathways.

A third model system, domestic pig skin, has a SC and an epidermal layer with approximately the same thicknesses as those of human skin. Qvist et al. (Eur. J. Pharm. Sci., 11:59-68, 2000) reported that domestic pig skin showed transdermal permeabilities for nicotine, salicylic acid and testosterone which correlated with human skin permeabilities. Thus, it is widely held that porcine epidermis is an acceptable skin and animal model to use in transdermal studies, particularly for investigating the transdermal permeation of lipophilic drugs (Dick, I. P., et al., J. Pharm. Pharmacol., 44:640-645, 1992).

For a molecule that is in solution, Fick's laws of diffusion govern its permeation from a drug-rich (or source) environment through a membrane into a drug-free (or sink) environment. Naik et al. (Int. J. Pharm., 90:129-140, 1993) showed that as the donor concentration of azidoprofen was increased from 6 to 15 μmol cm-3, the flux through hairless mouse skin increased from 0.236 to 0.649 μmol cm-2 hr-1.

The flux of a solute is proportional to the concentration gradient across the barrier phase. To acquire a maximum flux, the donor solution should be saturated; to maintain a saturated donor solution, the dissolution rate should be such that it does not become rate limiting. Any molecule passing through the skin, such as one being delivered from a transdermal system, will come into contact with regions that are both hydrophilic and lipophilic. Thus, the types of molecules which penetrate the skin most readily are those which are soluble in both oil and water (Hadgraft, J., Int. J. Pharm., 224:1-18, 2001). Water solubility of very lipophilic materials is typically very low, so the rate of partitioning from the stratum corneum to viable tissue can become rate limiting. In this respect, the partition coefficient is useful as a measuring tool, as it is a measure of the ability of a chemical to partition or separate between two immiscible phases (i.e., n-octanol and an aqueous phase).

There is little evidence to suggest that there are any active processes involved in skin permeation, supporting the theory that transdermal traffic is controlled by passive diffusion. Hadgraft (Int. J. Pharm., 224:1-18, 2001) postulates that there is likely some degree of correlation between the partition coefficient and in vivo percutaneous absorption, and that both reasonable lipid and water solubility appear necessary for the transdermal delivery of drugs.

Fick's laws of diffusion may be used to analyze permeation data. The first law is used to describe steady state diffusion and can be simplified to:

J=DKΔc/h Eqn. 1.1

where J is the flux per unit area, D is the diffusion coefficient in the skin, K is the skin-vehicle partition coefficient, Δc is the concentration difference across the skin and h is the diffusional pathlength. Under normal circumstances the applied concentration (cₐₚₚ) is very much larger than the concentration under the skin and Eqn. 1.1 is simplified to:

J=kₚ·cₐₚₚ Eqn. 1.2

where kₚ is a permeability coefficient (=KD/h) and is a heterogeneous rate constant having units, for example cm hr⁻¹. It is often difficult to separate K and D and their calculated magnitude will depend on h, the tortuosity of the intercellular channels in the skin.

The ionization constant, pKa, of a small molecule drug is an important parameter to consider in transdermal delivery. In conjunction with the pH of the vehicle, the pKa determines the proportion of the ionized and unionized drug species in the immediate vicinity of the skin. Since the aqueous solubility of ionized material is higher than unionized material, the maximum transdermal flux may occur at a pH where ionization is high.

In contrast, for peptide drugs, the isoelectric point (pI) is an important parameter to consider in transdermal delivery preparations. pI can usually be estimated by averaging the individual pKa values of free functional groups within the peptide or protein (Chiang, C.-H., et al., Drug Dev. Ind. Pharm., 24:431-438, 1998). Peptide drugs can be rendered positively or negatively charged by controlling the vehicle pH below or above the pI of the molecule. The solubility and charge density of peptide molecules increase when the solution pH is made either higher or lower than their isoelectric points as a result of protonation or dissociation of the various residues in these molecules (Chien, Y. W., et al., J. Pharm. Sci., 78:376-383, 1989).

Pro-drugs are therapeutically inactive derivatives of therapeutically active drugs that undergo a chemical or enzymatic transformation, typically in a biological environment, resulting in a therapeutically active drug (Chien, Y. W., Development and preclinical assessments of transdermal therapeutic systems, in Transdermal Controlled Systemic Medications, Chien, Y. W. (Ed.), Marcel Dekker Inc., New York, 1987). Upon absorption and penetration through the skin, the pro-drug is metabolized to generate the therapeutically active drug.

Peptide pro-drugs have long been associated with improvements in activity and in metabolic stability (see Borchardt, R. T., J. Control. Rel., 62:231-238, 1999; Dasgupta, P., et al., Br. J. Pharmacol., 129:101-109, 2000; Al-Obeidi, F., et al., J. Med. Chem., 35:118-123, 1992; Bundgaard, H., et al., Pharm. Res., 7:885-892, 1990). Borchardt (J. Control. Rel., 62:231-238, 1999) demonstrated that cyclic pro-drugs of opioid peptides demonstrated improved cell permeation characteristics as compared to the parent peptides, suggesting a possible future role in an oral formulation. A D(+)-maltose amadori derivative of the somatostatin analogue octreotide exhibited approximately ten times higher oral bioavailability than the parent compound while retaining selectivity, metabolic stability and duration of action (Albert, R., et al., Life Sci., 53:517-525, 1993). Chemical modification of insulin with palmitic acid increased lipophilicity, reduced degradation and resulted in increased transfer across the large intestinal mucous membrane (Hashizume, M., et al., J. Pharm. Pharmacol., 44:555-559, 1992). Improvement in the hydrophobicity and transdermal permeation of vasoactive intestinal peptide (VIP) was accomplished via N-acylation with stearic acid (Gozes, I., et al., J. Clin. Invest., 90:810-814, 1992). Conversely, increasing the hydrophobicity of gonadotropin releasing hormone (GnRH) via acylation of the lysine residue with various fatty acids led to a decrease in transdermal permeation (Yahalom, D., et al., Life Sci., 64:1543-1552, 1999).

Compounds that exhibit acid or base characteristics can participate in salt formation and the salt form of such compounds may exhibit altered lipophilic or stability profiles as compared to the parent compound. Peptide acetate salts, which are actual ion pairs, are usually highly hydrophilic with low partition coefficients (Adjei, A., et al. Int. J. Pharm., 90:141-149, 1993). Preparation of lipophilic peptide or protein salt forms may affect transdermal and transmucosal delivery.

Hydrophobic ion pairing (HIP) with ionic detergents has also been proposed by Meyer and Manning (Pharm. Res., 15:188-193, 1998) as a means of increasing protein solubility in organic phases, consequently increasing the partition coefficient. The HIP process has been exploited to purify protein mixtures, conduct enzymatic reactions in nonaqueous environments, increase structural stability, enhance bioavailability and prepare new dosage forms. The LHRH analog leuprolide was converted to an oleate salt and encapsulated in polymeric microspheres; while the microspheres made from the leuprolide oleate salt showed a release and stability profile similar to spheres made from leuprolide acetate salt, microspheres made with leuprolide oleate salt demonstrated a reduced burst release profile (Choi, S. H., et al., Int. J. Pharm., 203:193-202, 2000). Ion-pairing has also been investigated with diclofenac (Fini, A., et al., Int. J. Pharm., 187:163-173, 1999) and propanolol (Crowley, K. J., et al., J. Pharm. Sci., 88:586-591, 1999), the latter showing improved oral bioavailability as a laurate salt. Work by Valenta et al. (Int. J. Pharm., 197:77-85, 2000) on various lignocaine salts showed partition coefficients in general agreement with the lipophilicity of the counter ions used, although a correlation with transdermal steady state flux could not be found.

Successful transdermal drug delivery requires that the drug reach the skin surface at an adequate rate and partition favorably into the SC. The membrane-vehicle partition coefficient is influenced by the “escape tendency” of the drug from the vehicle; a higher escape tendency is reflected as an increased concentration gradient across the membrane and leads to an increased steady-state flux through the membrane. An alteration in a vehicle which increases solubility of the drug in that vehicle will decrease the rate of percutaneous absorption. Conversely, vehicle composition can be exploited to encourage supersaturation of the drug in the vehicle at lower concentrations. In supersaturated formulations, the thermodynamic activity of the drug in the vehicle is increased above unity, thus enhancing the driving force for drug delivery and increasing skin permeation. Moser et al. (J. Control. Rel., 73:245-253, 2001) have shown that a broad range of supersaturated formulations including hydrophilic, water-containing vehicles, a lipophilic oily composition and a cream, each prepared by different methods, enhanced the skin permeation of a lavendustin derivative, which is a lipophilic model compound.

Another factor that can affect the permeation of a weakly acidic or basic drug is the pH of an aqueous vehicle. For a weakly acidic drug, the unionized form will predominate at a low pH and thus the permeation rate will be maximized as passive diffusion across a lipid membrane (e.g. through the intercellular route of the stratum corneum) favors penetration of the unionized form of a molecule (Jack, L., et al., In vitro percutaneous absorption of salicylic acid: effect of pH, in Prediction of Percutaneous Penetration: Methods, Measurements, Modeling Vol. 2, Scott, R. C., Guy, R. H., Hadgraft, J. and Boddé, H. E. (Eds.), IBC Technical Services Ltd., London, 1991). However, the ionized form is also capable of permeating through the stratum corneum and hence, in order to maximize the flux through a membrane, vehicle pH should be such that both the concentrations of the unionized and the ionized forms are close to a maximum.

The vehicle selected should have no negative impact on drug stability. Recent development of a nicotine transdermal delivery system (Umprayn, K., Pharm. Tech. Eur., 12:54-59, 2000) showed that the drug was most stable in mineral oil under accelerated ageing conditions.

Finally, the vehicle itself can also have an effect on skin permeability in that it can affect skin hydration and may also act as an enhancer to transdermal penetration. In the case of lipophilic bases, hydrophilic bases and emulsifying bases, hydration of the skin occurs and water loss is prevented, leading to increased permeability.

An aqueous mixture is one example of a viable therapeutic transdermal delivery system. Ethanol has long been known to disrupt the barrier function of the SC via lipid extraction (Hadgraft, J., Int. J. Pharm., 224:1-18, 2001), however, it is considered a milder solvent when compared to other alcohols. Ethanol is currently used in at least three commercially available transdermal drug permeation systems for the delivery of fentanyl, estradiol and nitroglycerin. High concentrations of ethanol in the donor solution have been found to be beneficial for the transdermal delivery of melatonin (Oh, H.-J., et al., Int. J. Pharm. 212, 63-71, 2001; Kandimalla, K. K., et al., J. Control. Rel., 61:71-82, 1999). Although considered non-toxic, continual application of ethanol to the skin can induce eczema (Xu, P. et al., Crit. Rev. Ther. Drug Carrier Sys., 8:211-236, 1991).

Propylene glycol is another example of a vehicle useful in the preparation of formulations for transdermal drug delivery. It is thought to achieve penetration by moving gradually from a formulation into the intercellular spaces of the SC (Kandimalla, K. K., et al., J. Control. Rel., 61:71-82, 1999). DMSO has also been used to aid in the permeation of drugs across the skin. Its use at high concentrations in a topical diclofenac lotion has been reported by Hui et al. (Pharm. Res., 15:1589-1595, 1998).

In addition to the vehicle itself acting as a penetration enhancer by altering the barrier properties of the SC, additional components can be added to increase the passage or flux of a drug into and across the skin barrier. Chemical penetration enhancers (CPEs) are compounds that enhance the permeation of drugs across the skin by reversibly altering the physicochemical nature of the stratum corneum to reduce its diffusional resistance. CPEs also increase flux by increasing the partition coefficient of a drug into the skin and by increasing the thermodynamic activity of the drug in the vehicle.

A number of parameters should be considered when selecting a CPE (Kanikkannan, N., et al., Curr. Med. Chem., 7:593-608, 2000). A desirable CPE should be pharmacologically inert, possessing no action at receptor sites anywhere in the body. A flux enhancer should be non-toxic, non-irritating and non-allergenic as well as inexpensive, relatively odorless, tasteless and colorless with a suitable spread and skin feel. The CPE should alter the barrier function of the skin in one direction only; endogenous materials should not be lost to the environment by diffusion out of the skin. Flux enhancers should alter the permeability of the SC and modify the partitioning between this outer skin layer and the underlying viable tissue. The onset of action of the CPE should be rapid, and duration of activity should be predictable and suitable for the drug used. Upon removal of the enhancer, the SC should immediately and fully recover its normal barrier property. The enhancer should be chemically and physically compatible with all drugs and adjuvants to be formulated in topical preparations and devices, and should readily formulate into dermatological preparations, transdermal devices and skin adhesives. If the enhancer is a liquid and is to be used at high volume fractions, it should be a suitable solvent for the drug.

Among the CPEs most widely investigated are long chain fatty acids (most commonly oleic acid), fatty alcohols (mainly ethanol), terpenes (e.g, cineole), dimethylsulfoxide (and its related aprotic solvents dimethylformamide and dimethylacetamide), urea, azone, propylene glycol and sodium lauryl sulfate. Based on the chemical structures of these various enhancers such as chain length, polarity, level of unsaturation and presence of special groups such as ketones, their interaction with the stratum corneum may vary considerably, resulting in significant differences in the penetration enhancement of various drugs.

The unsaturated fatty acid oleic acid is one example of a CPE. Oleic acid is believed to increase skin permeability by disruption of the densely packed lipids that fill the extracellular spaces of the SC (Kanikkannan, N., et al., Curr. Med. Chem., 7:593-608, 2000). Ongpipattanakul et al. (Pharm. Res., 8; 350-354, 1991) have provided evidence that oleic acid may exist as a liquid within the lipids at physiological temperature. Oleic acid assisted transdermal transport is believed to occur via the formation of permeable interfacial defects within the SC lipid bilayers which decrease either the diffusional pathlength or the resistance to flux. Recent examples of the flux enhancing effect of oleic acid can be found for piroxicam (Santoyo, S. et al., Eur. J. Pharm. Biopharm., 50:245-250, 2000), Tranilast (Murakami, T., et al., J. Pharm. Pharmacol., 50:49-54, 1998) and melatonin (Kanikkannan, N., et al., Curr. Med. Chem., 7:593-608, 2000).

Another penetration enhancer, the terpene 1,8-Cineole (also known as eucalyptol) is used in various products for its fragrance, counterirritant and antipruritic effects. Terpenes in general act by disrupting the lipid structure of the SC, thereby increasing the diffusion coefficient of a drug in the membrane (Kanikkannan, N., et al., Curr. Med. Chem., 7:593-608, 2000). Cal et al. (Int. J. Pharm., 224:81-88, 2001) report a relatively short lag time is characteristic of all terpenes as penetration enhancers. Tamoxifen (Gao, S., et al., J. Control. Rel., 51:193-199, 1998), LHRH (Bhatia, K. S., et al., Pharm. Res., 15:1857-1862, 1998), piroxicam (Doliwa, A., et al., Int. J. Pharm., 229:37-44, 2001) and diclofenac (Obata, Y., et al., Drug Des. Disc., 8:137-144, 1991) have all shown improvements in transdermal flux when cineole is incorporated into an ethanol-based donor solution.

Long chain fatty alcohols also function as CPEs. For longer chain saturated fatty alcohols, a parabolic relationship between the carbon chain length of the alcohol and the permeation enhancement of melatonin has been reported (Andega, S., et al., J. Control. Rel., 77:17-25, 2001) where maximum permeation was achieved using decanol (C10) and dodecanol (C12). Kanikkannan et al. (Curr. Med. Chem., 7:593-608, 2000) theorize that this may be due to the fact these chain lengths correspond to the chain length of the steroid nucleus of cholesterol and the fatty alcohols may act by disrupting ceramide-cholesterol or cholesterol-cholesterol interactions in the skin.

Although the SC itself acts as a highly efficient rate controlling membrane, additional permeability control introduced by a transdermal patch and choice of vehicle aid in ensuring that systemic concentrations remain within the desired therapeutic levels. If the drug is present at a concentration above its saturation concentration, whereby excessive drug allows a constant concentration gradient to be obtained, zero-order drug release can be achieved.

In general, a transdermal patch (TDP), also referred to herein as a transdermal therapeutic system (TTS), comprises a drug-containing reservoir, an adjacent release rate-controlling membrane and some form of an adhesive layer to affix the delivery system to the skin. A patch size of approximately 50 cm2 allows for maximum dispersal area, patient comfort and cosmetic appeal, delivering up to approximately 5-20 mg per day. Once-a-day, twice-weekly or seven-day applications are best correlated with human routines. Passive transdermal patches typically fall within one of three delivery systems: a membrane permeation-controlled system, an adhesive dispersion-type system, or a matrix diffusion-controlled system (Chien, Y. W., Development and preclinical assessments of transdermal therapeutic systems, in Transdermal Controlled Systemic Medications, Chien, Y. W. (Ed.), Marcel Dekker Inc., New York, 1987).

Membrane permeation controlled TDPs comprise a drug-loaded matrix or reservoir completely covered by a rate-controlling membrane; the drug-releasing surface is coated with an adhesive film which is protected by a release liner. In some cases, the adhesive layer contains a “priming” dose of the drug. Since the adhesive layer is positioned across the drug-releasing surface of the device, the drug must be able to diffuse through the adhesive without adversely affecting adhesive properties. The nature of the membrane permeation controlled TDP requires an adhesive polymer that is physicochemically compatible with the drug of choice, with little to no effect upon the delivery rate of the drug out of the device.

In the reservoir, the drug can either be dispersed homogeneously in a solid polymer matrix, suspended in a viscous liquid medium or any other suitable vehicle with or without CPEs. The rate-controlling membrane can be a microporous, a polymeric or any other suitable membrane. It is possible to alter the rate of drug release from the system by varying the polymer composition, the permeability coefficient and/or the thickness of the rate-controlling membrane and adhesive.

Adhesive dispersion-type TDPs differ from membrane permeation controlled TDPs in that the drug is directly loaded or dispersed into the adhesive polymer layer. Long-term compatibility between the adhesive, the drug and any excipients introduced to the formulation is required. No rate-controlling membrane is present but there may often be a thin layer of non-medicated, rate-controlling adhesive of a specific permeability which is designed to be in direct contact with the skin. Adhesive dispersion-type TDPs can be further modified to have the drug loading level varied at increments to form a gradient of drug reservoir across the multilaminate adhesive layers.

In a third type of TDP system, a matrix diffusion controlled device, the adhesive is present as a film around the edge of the TDP and does not come into direct contact with the drug. The drug is homogeneously dispersed in a hydrophilic, lipophilic or other suitable polymer matrix, which is then molded into a medicated disc with a defined surface area and controlled thickness.

Pressure-sensitive adhesives (PSAs) are a critical component in transdermal drug delivery systems. In addition to possessing exceptional skin adhesion properties, the adhesives must be extremely stable, consistent from lot to lot and compatible with the drug contained within the TDP. Standard functional properties such as tack, adhesion, release force and cohesive strength must be demonstrated on the highly variable skin substrate over a broad range of temperature, relative humidity, immersion times, application times and mechanical movements. Skin irritation and sensitization properties of the adhesive, both with and without the drug, must also be understood and managed (Robertson, M., Pharm. Tech. Eur., 13:20-24, 2001).

Useful PSAs include, but are not limited to, acrylic, polyisobutylene (PIB) and silicone-type adhesives. Acrylic type PSAs are readily cross-linked, which can improve co-adhesive properties if degraded by the drug, enhancers or solvents. PIB cohesive properties may be preferred for drugs with low solubility and polarity. Silicones generally offer the highest drug diffusion rates; low-silinol silicone formulations are particularly amine-compatible (Robertson, M., Pharm. Tech. Eur., 13:20-24, 2001). Adhesive selection is based on a number of factors including the patch design, drug formulation, adhesion properties, skin compatibility and determining the rate at which the drug will migrate through the adhesive.

Examples of transdermal patches include, but are not limited to the following. U.S. Pat. No. 4,668,232 describes a matrix for a transdermal patch, which comprises a reservoir layer comprising a water-swellable polymeric matrix composed of an adhesive material, and a drug that is partially or wholly soluble in the adhesive material. The inclusion of the water-swellable polymer is alleged to increase the release rate of the drug from the matrix. U.S. Pat. No. 5,230,898 describes a transdermal patch comprising a matrix composed of a water-insoluble material that contains islands of solid particles of a drug in a water-soluble/swellable polymer and an underlayer that controls the amount of water vapor passing from the skin to the matrix. The matrix is said to be activated by water vapor from the skin. U.S. Pat. No. 4,559,222 describes a transdermal matrix-type patch in which the matrix is composed of a mixture of mineral oil, polyisobutylene adhesive and colloidal silicon dioxide. The addition of the silicon dioxide allegedly affects the flow characteristics of the mineral oil-polyisobutylene mix.

A number of mathematical models have been developed to describe percutaneous absorption kinetics. In general, most of these models have used either diffusion-based or compartmental equations. Many of the current models for transdermal delivery assume that the drug diffusivity in the SC remains constant during transport and more generally that this membrane is unaffected by the formulation. However, one of the principle aims of a transdermal formulation is to maximise delivery, whether by occluding the skin and changing the membrane's properties by increasing hydration, or by releasing vehicle components that enhance drug penetration. Therefore barrier function as well as the membrane itself is changed by the action of the formulation.

The rate-limiting step in transdermal delivery is generally recognized to be diffusion of the drug through the SC via a lipidic intercellular pathway. The rate of transdermal delivery can be controlled by the SC or the delivery device used, or a combination of both. The fractional rate control provided by the device can be described by:

FD=Mₜₒₜₐₗ/M_{device} Eqn. 1.3

where Mₜₒₜₐₗ/M_{device} is the amount of drug released when a device is in contact with the skin divided by the amount released for a given time from that device into an aqueous sink (i.e. in the absence of the SC; Kalia, Y. N., et al., Adv. Drug. Del. Rev., 48:159-172, 2001)). The parameter is unitless and if FD=1, then drug delivery is entirely controlled by the device, whereas FD values less than one indicate some contribution from the skin towards control. The fractional rate control by the skin may be further defined as:

FS=1−FD=1−Mₜₒₜₐₗ/M_{device} Eqn. 1.4

Many ointments contain a finely divided drug suspension whereby the drug is at maximum thermodynamic activity. The scenario whereby the rate determining step is transport within a vehicle containing a suspension of the drug was first described by Higuchi (J. Soc. Cosmet. Chem., 11:85-97, 1960). Under quasi steady-state conditions the amount of drug transported per unit area of skin (Q) at a given time (t) is defined as:

Q=\sqrt{DtC_{S}(2C₀−C_{S})} Eqn. 1.5

where C₀ and Cₛ are the original drug concentration in the vehicle and the solubility limit of the drug in the vehicle respectively, and D is the diffusivity of the drug in the vehicle. The equation can be differentiated to give an instantaneous release rate at time t:

dQ/dt=½\sqrt{DC_{S}(2C₀−C_{S})/t} Eqn. 1.6

The Higuchi model assumes that C₀>>Cₛ and so Equations 1.5 and 1.6 can be simplified to:

$$\begin{matrix}Q = \sqrt{2 {C}_{0} {DtC}_{S}} \\ and & Eqn . 1.7 \\ \frac{d Q}{d t} = \sqrt{\frac{{C}_{0} D {C}_{S}}{2 t}} & Eqn . 1.8\end{matrix}$$

Bunge (J. Control. Rel., 52:141-148, 1998) modified the Higuchi equation to determine the fraction of the total drug mass absorbed at time t, assuming a linear concentration profile in the dissolved region (also an assumption of the Higuchi model). Thus,

$$\frac{{M}_{t}}{{M}_{0}} = \sqrt{\frac{2 DtR ( 1 - R / 2 )}{{L}^{2}}} \qquad Eqn . 1.9$$

where R=Cₛ/C₀ and L is the thickness of the vehicle. The exact solution to the equation, whereby a linear concentration gradient in the region adjacent to the formulation-membrane interface is not assumed, is a lot more complicated. This was determined by Paul and McSpadden (J. Membr. Sci. 1:33-48, 1976):

$$\frac{{M}_{t}}{{M}_{0}} = 2 ( 1 - R β {e}^{{β}^{2}} \sqrt{\frac{Dt}{{L}^{2}}} ) \qquad Eqn . 1.10$$

where β is described by:

$$\sqrt{π} β {e}^{{β}^{2 }} erf ( β ) = \frac{R}{1 - R} \qquad Eqn . 1.11$$

and erf (β) is the error function of β. It was shown that both the Higuchi approximation and its exact solution predict that the normalized mass fraction released (Mₜ/M₀/(2RDt/L²)^{1/2}) has an almost linear dependence on R. However, the Higuchi approximation underestimates the total mass released over the range of R-values (0<R<1) due to the assumption of a linear concentration profile in the dissolved region. Bunge further proposed a simple modification of the basic Higuchi equation to compensate for this approximation without resorting to the full analytical solution:

$$\frac{{M}_{t}}{{M}_{0}} = \sqrt{\frac{2 DtR ( 1 - \frac{R ( π - 2 )}{π} )}{{L}^{2}}} \qquad Eqn . 1.12$$

This was used along with the unmodified Higuchi equation to predict Mt and it was found that for R=1, (i.e Cₛ=C₀) the unmodified equation gave only a 12% error (as compared to the exact solution) with respect to the prediction of the amount of drug released. This was unexpected as the Higuchi equation should breakdown at Cₛ=C₀ due to the assumption that C₀>>Cₛ. The modified Higuchi equation fared even better, with only a 0.5% deviation from the exact solution. Bunge showed that the unmodified equation tended to overestimate diffusivity by 27%, while his modified version showed a maximum deviation of only 2%.

These results led Bunge to conclude that despite the assumptions made during the derivation of the Higuchi equation, it was still capable of predicting reasonably accurately the amount of drug released from a formulation assuming that R<0.5 (i.e. the starting drug concentration in the vehicle is at least twice its saturation limit). Additionally, for larger R-values (whereby the starting concentration moves towards and below the saturation limit) the modified Higuchi equation showed excellent accuracy in predicting drug release and was a better method for calculating diffusivities.

The use of ointments and gels can result in the application of layers having different thicknesses. Guy and Hadgraft (Int. J. Pharm., 6:321-332, 1980) developed a mathematical model to investigate the effect of varying application thickness on drug release and uptake by the skin. The rate-controlling layer would be determined by the relative rates of transport in the formulation and in the SC, as evidenced by values of D/L² in the respective media. Thus the total amount of drug (Mₜ) that passes through the SC of thickness Lₛ (the subscripts ‘O’ and ‘S’ are used to denote the ointment and the SC respectively) during the period t was given by:

$${M}_{t} = - {D}_{S} A {\int }_{0}^{t} {( \frac{d {C}_{S}}{d x} )}_{x = {L}_{s}} d t \qquad Eqn . 1.13$$

Various diffusion equations describing the above system were solved using a series of dimensionless variables:

$$μ = \frac{C}{{C}_{∞}}$$

$$λ = \frac{{D}_{S} / {L}_{S}}{{D}_{O} / {L}_{O}}$$

$$ρ = \frac{{D}_{O} / {L}_{O}^{2}}{{D}_{S} / {L}_{S}^{2}}$$

$$τ = \frac{{D}_{S} t}{{L}_{S}^{2}}$$

Assuming fast interfacial kinetics between the formulation and the SC, the following diffusion equations based upon the normalized parameters describe the mass transfer in the vehicle and the SC respectively:

$$\begin{matrix}\frac{δ {μ}_{O}}{δ τ} = ρ ( \frac{{δ}^{2} {μ}_{0}}{δ {χ}^{′2}} ) & Eqn . 1.14 \\ \frac{δ {μ}_{S}}{δ τ} = λ ( \frac{{δ}^{2} {μ}_{S}}{{δχ}^{2}} ) & Eqn . 1.15\end{matrix}$$

where χ′ (=x/L_{O}) and χ(=x/L_{S}) are the normalized pathlengths in the formulation and the skin. The boundary condition between the formulation and the SC is described by:

$${( \frac{δ {μ}_{O}}{δ {χ}^{′}} )}_{O} = - {λ ( \frac{{δμ}_{S}}{δχ} )}_{O} \qquad Eqn . 1.16$$

The Mₜ equation was also rewritten in terms of dimensionless parameters:

$${M}_{t} = {AL}_{S} {C}_{O} {\int }_{0}^{τ} ( \frac{{δμ}_{S}}{δχ} ) δ τ \qquad Eqn . 1.17$$

A complicated Laplace transform solution to the diffusion equations resulted in an expression for Mₜ containing cos h, tan h and cotan h functions (hyperbolic cosine, tangent and cotangent, respectively). Limiting approximations for short and long-time exposures were used to simplify the general expression and yield analytical solutions. Thus, at the short time limit, inversion of the transformed expression followed by substitution of the absolute parameters and simplification gave:

$$\frac{{M}_{t}}{{M}_{∞ }} = \frac{8 ( \frac{{L}_{S}}{{L}_{O}} )}{K + \sqrt{\frac{{D}_{S}}{{D}_{O}}}} \sqrt{\frac{1}{π} {( \frac{{D}_{S} t}{{L}_{S}^{2}} )}^{3}} exp ( - \frac{{L}_{S}^{2}}{4 {D}_{S} t} ) \qquad Eqn . 1.18$$

where K=μO₀/μS₀ representing the partition coefficient between the formulation and the membrane.

Thus, at short times, the key parameters are the formulation/membrane partition coefficient, the diffusion coefficients of the drug in the formulation and the SC, and the ratio of the diffusion pathlengths in the formulation and the SC. Guy and Hadgraft stated that, since Ds<<Do and K is also small since most formulations are designed to favor drug entry into the SC, it is not possible to determine which factor(s) will be dominant. For long-time exposures Eqn 1.19 holds, assuming steady state flux.

$$\frac{{M}_{t}}{{M}_{∞}} = ( 1 - exp ( - \frac{{D}_{S } t}{{KL}_{O} {L}_{S}} ) ) \qquad Eqn . 1.19$$

In other words, the diffusional pathlength in the formulation, Lo, plays a role in drug release. As the thickness of the applied layer is increased, a smaller proportion of the total amount of drug within the layer is released after a given time.

Mathematical models describing drug release from a solution are more complex because they must incorporate the changing activity of the drug as it is depleted in the formulation. The greater the disparity between the characteristic diffusion times through the vehicle (Lᵥₑₕ²/Dᵥₑₕ) and the skin (L_{SC}²/KD_{SC}), the greater the degree of control residing within the SC. If the partition coefficient is large this can reduce the rate of SC control, highlighting the effect of interfacial transfer kinetics in multilayer delivery systems. In systems where there is a significant degree of rate control by the SC (e.g. where L_{SC}²/KD_{SC}>>10 Lᵥₑₕ²/Dᵥₑₕ), then the fraction of drug (Mₜ/M_{∞}) released from a finite slab of thickness Lᵥₑₕ is:

$$\frac{{M}_{t}}{{M}_{∞}} = {\sum }_{n = 1}^{∞} \frac{2 K}{{L}_{veh}} \frac{( 1 - exp ( - {D}_{S C} {α}_{n}^{2} t ) )}{[ {L}_{SC} \{ {α}_{n}^{2} + {( \frac{K}{{L}_{veh}} )}^{2} \} + ( \frac{K}{{L}_{veh}} ) ] cos ( {α}_{n} {L}_{SC} )} \qquad Eqn . 1.20$$

and α is derived from αnlSCtan(αnlSC)=Kl_{SC}/Lᵥₑₕ.

This delivery scenario is complicated by the fact that depletion of the drug in the vehicle can occur before significant amounts have passed through the SC and entered into the underlying tissues. Thus, the thermodynamic driving force for drug delivery is decreasing throughout the delivery process. The average applied thickness of a topical formulation is thought to be ˜20 μm (Kalia, Y. N., et al., Adv. Drug. Del. Rev., 48:159-172, 2001), hence, passage of only a small quantity of drug into the SC might set up a steep concentration gradient in the vehicle.

Classification of therapeutic transdermal systems according to drug release mechanism depends, in the first instance, on whether the drug is entirely dissolved in the formulation or exists in the solid form. Formulations that contain the drug above the saturation concentration in the form of a suspension will give rise to drug release profiles that follow the Higuchi model presented here.

For system designs where drug is present below its saturation concentration, the drug release profile will be based on the membrane models proposed by Hadgraft (Int. J. Pharm., 2:177-194, 1979) for diffusion from a slab, taking into account the multilaminar nature of the delivery device and the possible influence of any interfacial transfer kinetics. The presence of a rate-controlling membrane incorporated into the patch will ensure the input rate is determined by the drug delivery device otherwise the SC will function, to some extent, as the rate-controlling membrane.

Peptides Suitable for Use with the Invention

A) Somatostatin, a tetradecapeptide discovered by Brazeau et al. (Science, 179:77-79, 1973), has been shown to have potent inhibitory effects on various secretory processes and cell proliferation in normal and neoplastic human tissues such as pituitary, pancreas and the gastrointestinal tract. Somatostatin also acts as a neuromodulator in the central nervous system. These biological effects of somatostatin, all inhibitory in nature, are elicited through a series of G protein coupled receptors, of which five different subtypes have been characterized, hereinafter referred to as “SSTR-1”, “SSTR-2”, “SSTR-3”, “SSTR-4” and “SSTR-5” for each of the five receptors or generally and/or collectively as “SSTR” (Patel, Y. C., Front. Neuroendocrinol., 20:157-98, 1999; and Zatelli, M. C. et al., J. Endocrinol. Invest., 27 Suppl(6):168-70, 2004). These five subtypes have similar affinities for the endogenous somatostatin ligands but have differing distribution in various tissues. Somatostatin binds to the five distinct receptor subtypes with relatively high and equal affinity for each.

As is well known to those skilled in the art, SRIF and analogs thereof are useful in the treatment of a great variety of diseases and/or conditions. An exemplary, but by no means exhaustive, list of such diseases and/or conditions would include: Cushings' Syndrome (see Clark, R. V. et al, Clin. Res., 38:943A, 1990); gonadotropinoma (see Ambrosi, B., et al., Acta Endocr. (Copenh.), 122:569-576, 1990); hyperparathyroidism (see Miller, D., et al., Canad. Med. Ass. J., 145:227-228, 1991); Paget's disease (see Palmieri, G. M. A., et al., J. of Bone and Mineral Research, 7(Suppl. 1):S240 (Abs. 591), 1992); VIPoma (see Koberstein, B., et al., Z. Gastroenterology, 28:295-301, 1990; Christensen, C., Acta Chir. Scand. 155:541-543, 1989); nesidioblastosis and hyperinsulinism (see Laron, Z., Israel J. Med. Sci., 26:1-2, 1990; Wilson, D. C., Irish J. Med. Sci., 158:31-32, 1989; Micic, D., et al., Digestion, 16, (Suppl. 1.70)Abs. 193, 1990); gastrinoma (see Bauer, F. E., et al., Europ. J. Pharmacol., 183:55, 1990); Zollinger-Ellison Syndrome (see Mozell, E., et al., Surg. Gynec. Obstet., 170:476-484, 1990); hypersecretory diarrhea related to AIDS and other conditions (due to AIDS, see Cello, J. P., et al., Gastroenterology, 98(No. 5, Part 2) Suppl.: A163, 1990; due to elevated gastrin-releasing peptide, see Alhindawi, R., et al., Can. J. Surg., 33:139-142, 1990; secondary to intestinal graft vs. host disease, see Bianco J. A., et al., Transplantation, 49:1194-1195, 1990; diarrhea associated with chemotherapy, see Petrelli, N., et al., Proc. Amer. Soc. Clin. Oncol., 10:138, Abstr. No. 417, 1991); irritable bowel syndrome (see O'Donnell, L. J. D., et al., Aliment. Pharmacol. Therap., 4.:177-181, 1990); pancreatitis (see Tulassay, Z., et al., Gastroenterology, 98(No. 5, Part 2) Suppl., A238, 1990); Crohn's Disease (see Fedorak, R. N., et al., Can. J. Gastroenterology, 3:53-57, 1989); systemic sclerosis (see Soudah, H., et al., Gastroenterology, 98(No. 5, Part 2) Suppl., A129, 1990); thyroid cancer (see Modigliani, E., et al., Ann., Endocr. (Paris), 50:483-488, 1989); psoriasis (see Camisa, C., et al., Cleveland Clinic J. Med., 57:71-76, 1990); hypotension (see Hoeldtke, R. D., et al., Arch. Phys. Med. Rehabil., 69:895-898, 1988; Kooner, J. S., et al., Brit. J. Clin. Pharmacol., 28:735P-736P, 1989); panic attacks (see Abelson, J. L., et al., Clin. Psychopharmacol., 10:128-132, 1990); sclerodoma (see Soudah, H., et al., Clin. Res., 39:303A, 1991); small bowel obstruction (see Nott, D. M., et al., Brit. J. Surg., 77:A691, 1990); gastroesophageal reflux (see Branch, M. S., et al., Gastroenterology, 100(No. 5, Part 2 Suppl.):A425, 1991); duodenogastric reflux (see Hasler, W., et al., Gastroenterology, 100(No. 5, Part 2, Suppl.):A448, 1991); Graves' Disease (see Chang, T. C., et al., Brit. Med. J., 304:158, 1992); polycystic ovary disease (see Prelevic, G. M., et al., Metabolism Clinical and Experimental, 41(Suppl. 2):76-79, 1992); upper gastrointestinal bleeding (see Jenkins, S. A., et al., Gut., 33:404-407, 1992; Arrigoni, A., et al., American Journal of Gastroenterology, 87:1311, (Abs. 275), 1992); pancreatic pseudocysts and ascites (see Hartley, J. E., et al., J. Roy. Soc. Med., 85:107-108, 1992); leukemia (see Santini, et al., 78(Suppl. 1):429A (Abs. 1708), 1991); meningioma (see Koper, J. W., et al., J. Clin. Endocr. Metab., 74:543-547, 1992); and cancer cachexia (see Bartlett, D. L., et al., Surg. Forum., 42:14-16, 1991).

Binding to the particular subtypes of somatostatin receptors has been associated with the treatment of various conditions and/or diseases. For example, the inhibition of growth hormone has been attributed to SSTR-2 (Raynor, et al., Molecular Pharmacol., 43:838, 1993; Lloyd, et al., Am. J. Physiol., 268:G102, 1995) while the inhibition of insulin has been attributed to SSTR-5. Activation of SSTR-2 and SSTR-5 has been associated with growth hormone suppression and more particularly GH secreting adenomas (acromegaly) and TSH secreting adenomas. Activation of SSTR-2 but not SSTR-5 has been associated with treating prolactin secreting adenomas.

There is evidence that somatostatin regulates cell proliferation by arresting cell growth via SSTR-1, -2, -4 and -5 subtypes (Buscail, L. et al., Proc. Natl. Acad. Sci. USA, 92:1580-4, 1995; Buscail, L. et al., Proc. Natl. Acad. Sci. USA, 91:2315-9, 1994; Florio, T. et al., Mol. Endocrinol., 13:24-37, 1999; Sharma, K. et al., Mol. Endocrinol., 13:82-90, 1999) or by inducing apoptosis via SSTR-3 subtype (Sharma, K. et al., Mol. Endocrinol., 10:1688-96, 1996). Somatostatin and various analogues have been shown to inhibit normal and neoplastic cell proliferation in vitro and in vivo (Lamberts, S. W. et al., Endocrin. Rev., 12:450-82, 1991) via specific somatostatin receptors (Patel, Y. C., Front Neuroendocrin., 20:157-98, 1999) and possibly different post-receptor actions (Weckbecker, G. et al., Pharmacol. Ther., 60:245-64, 1993; Bell, G. I. and Reisine, T., Trends Neurosci., 16:34-8, 1993; Patel, Y. C. et al., Biochem. Biophys. Res. Comm., 198:605-12, 1994; Law, S. F. et al., Cell Signal, 7:1-8, 1995). In addition, there is evidence that distinct SSTR subtypes are expressed in normal and neoplastic human tissues (Virgolini, I. et al., Eur. J. Clin. Invest., 27:645-7, 1997) conferring different tissue affinities for various somatostatin analogues and variable clinical response to their therapeutic effects.

As various somatostatin receptors have been isolated, e.g., SSTR-1, SSTR-2, SSTR-3, SSTR-4, and SSTR-5, a somatostatin agonist may be one or more of an SSTR-1 agonist, SSTR-2 agonist, SSTR-3 agonist, SSTR-4 agonist or a SSTR-δ agonist.

What is meant by an SSTR-1 receptor agonist (i.e., SSTR-1 agonist) is a compound which has a high binding affinity (e.g., Kᵢ of less than 100 nM or preferably less than 10 nM or less than 1 nM) for SSTR-1 (e.g., as defined by the receptor binding assay in U.S. Pat. No. 7,084,117 incorporated herein by reference in its entirety). What is meant by an SSTR-1 receptor selective agonist is an SSTR-1 receptor agonist that has a higher binding affinity (i.e., lower Kᵢ) for SSTR-1 than for another receptor, i.e., SSTR-2, SSTR-3, SSTR-4 or SSTR-5.

What is meant by an SSTR-2 receptor agonist is a somatostatin agonist which has a high binding affinity (e.g., Kᵢ of less than 100 nM or preferably less than 10 nM or less than 1 nM) for SSTR-2 (e.g., as defined by the receptor binding assay in U.S. Pat. No. 7,084,117 incorporated herein by reference in its entirety). What is meant by an SSTR-2 receptor selective agonist is an SSTR-2 receptor agonist that has a higher binding affinity (i.e., lower Kᵢ) for SSTR-2 than for any other somatostatin receptor i.e., SSTR-1, SSTR-3, SSTR-4 or SSTR-5.

What is meant by an SSTR-3 receptor agonist is a somatostatin agonist which has a high binding affinity (e.g., Kᵢ of less than 100 nM or preferably less than 10 nM or less than 1 nM) for SSTR-3 (e.g., as defined by the receptor binding assay in U.S. Pat. No. 7,084,117 incorporated herein by reference in its entirety). What is meant by an SSTR-3 receptor selective agonist is an SSTR-3 receptor agonist that has a higher binding affinity (i.e., lower Kᵢ) for SSTR-3 than for any other somatostatin receptor i.e., SSTR-1, SSTR-2, SSTR-4 or SSTR-5.

What is meant by an SSTR-4 receptor agonist is a somatostatin agonist which has a high binding affinity (e.g., Kᵢ of less than 100 nM or preferably less than 10 nM or less than 1 nM) for SSTR-4 (e.g., as defined by the receptor binding assay in U.S. Pat. No. 7,084,117 incorporated herein by reference in its entirety). What is meant by an SSTR-4 receptor selective agonist is an SSTR-4 receptor agonist that has a higher binding affinity (i.e., lower Kᵢ) for SSTR-4 than for any other somatostatin receptor i.e., SSTR-1, SSTR-2, SSTR-3 or SSTR-5.

What is meant by an SSTR-5 receptor agonist is a somatostatin agonist which has a high binding affinity (e.g., Kᵢ of less than 100 nM or preferably less than 10 nM or less than 1 nM) for SSTR-5 (e.g., as defined by the receptor binding assay in U.S. Pat. No. 7,084,117 incorporated herein by reference in its entirety). What is meant by an SSTR-5 receptor selective agonist is an SSTR-5 receptor agonist that has a higher binding affinity (i.e., lower Kᵢ) for SSTR-5 than for any other somatostatin receptor i.e., SSTR-1, SSTR-2, SSTR-3 or SSTR-4.

Some somatostatin agonist compounds exhibit high binding affinities for two, or even three, somatostatin receptors as compared to other somatostatin receptors. Such somatostatin agonists are also classified as a somatostatin agonist wherein the compound has a high binding affinity (e.g., Kᵢ of less than 100 nM or preferably less than 10 nM or less than 1 nM) for two (or three) different somatostatin receptors (e.g., as defined by the receptor binding assay in U.S. Pat. No. 7,084,117 incorporated herein by reference in its entirety). Thus, what is meant by an SSTR-5 and SSTR-2 receptor agonist is a receptor agonist that has a higher binding affinity (i.e., lower Kᵢ) for SSTR-5 and for SSTR-2 than for other somatostatin receptors, i.e., SSTR-1, SSTR-3 or SSTR-4.

In addition to the commercially available Octreotide® and Lanreotide™, a large number of second generation somatostatin analogs have been proposed for use as therapeutic agents to detect and/or treat cancer and other somatostatin-responsive disease states. Such second generation somatostatin analogs are described in:

U.S. Pat. No. 3,997,517 (1976);
U.S. Pat. No. 4,000,259 (1976);
U.S. Pat. No. 4,011,182 (1977);
U.S. Pat. No. 4,077,952 (1978);
U.S. Pat. No. 4,098,782 (1978);
U.S. Pat. No. 4,100,153 (1978);
U.S. Pat. No. 4,104,267 (1978);
U.S. Pat. No. 4,105,603 (1978);
U.S. Pat. No. 4,115,554 (1978);
U.S. Pat. No. 4,122,077 (1978);
U.S. Pat. No. 4,123,425 (1978);
U.S. Pat. No. 4,130,554 (1978);
U.S. Pat. No. 4,133,782 (1979);
U.S. Pat. No. 4,139,526 (1979);
U.S. Pat. No. 4,140,767 (1979);
U.S. Pat. No. 4,145,337 (1979);
U.S. Pat. No. 4,146,612 (1979);
U.S. Pat. No. 4,159,263 (1979);
U.S. Pat. No. 4,161,521 (1979);
U.S. Pat. No. 4,162,248 (1979);
U.S. Pat. No. 4,185,010 (1980);
U.S. Pat. No. 4,190,575 (1980);
U.S. Pat. No. 4,190,648 (1980);
U.S. Pat. No. 4,191,754 (1980);
U.S. Pat. No. 4,204,990 (1980);
U.S. Pat. No. 4,209,426 (1980);
U.S. Pat. No. 4,211,693 (1980);
U.S. Pat. No. 4,215,039 (1980);
U.S. Pat. No. 4,224,190 (1980);
U.S. Pat. No. 4,235,886 (1980);
U.S. Pat. No. 4,238,481 (1980);
U.S. Pat. No. RE30,548 (1981);
U.S. Pat. No. 4,261,885 (1981);
U.S. Pat. No. 4,280,953 (1981);
U.S. Pat. No. 4,282,143 (1981);
U.S. Pat. No. 4,291,022 (1981);
U.S. Pat. No. 4,310,518 (1982);
U.S. Pat. No. 4,316,890 (1982);
U.S. Pat. No. 4,316,891 (1982);
U.S. Pat. No. 4,328,214 (1982);
U.S. Pat. No. 4,358,439 (1982);
U.S. Pat. No. 4,360,516 (1982);
U.S. Pat. No. 4,369,179 (1983);
U.S. Pat. No. 4,395,403 (1983);
U.S. Pat. No. 4,427,661 (1984);
U.S. Pat. No. 4,428,942 (1984);
U.S. Pat. No. 4,435,385 (1984);
U.S. Pat. No. 4,853,731 (1984);
U.S. Pat. No. 4,485,101 (1984);
U.S. Pat. No. 4,486,415 (1984);
U.S. Pat. No. 4,505,897 (1985);
U.S. Pat. No. 4,508,711 (1985);
U.S. Pat. No. 4,522,813 (1985);
U.S. Pat. No. 4,585,755 (1986);
U.S. Pat. No. 4,603,120 (1986);
U.S. Pat. No. 4,611,054 (1986);
U.S. Pat. No. 4,612,366 (1986);
U.S. Pat. No. 4,650,787 (1987);
U.S. Pat. No. 4,663,435 (1987);
U.S. Pat. No. 4,684,620 (1987);
U.S. Pat. No. 4,703,034 (1987);
U.S. Pat. No. 4,725,577 (1988);
U.S. Pat. No. 4,728,638 (1988);
U.S. Pat. No. 4,803,261 (1989);
U.S. Pat. No. 4,816,438 (1989);
U.S. Pat. No. 4,837,303 (1989);
U.S. Pat. No. 4,853,371 (1989);
U.S. Pat. No. 4,871,717 (1989);
U.S. Pat. No. 4,897,445 (1990);
U.S. Pat. No. 4,904,642 (1990);
U.S. Pat. No. 5,059,653 (1991);
U.S. Pat. No. 5,506,339 (1996);
U.S. Pat. No. 5,552,520 (1996);
U.S. Pat. No. 5,595,760 (1997);
U.S. Pat. No. 5,597,894 (1997);
U.S. Pat. No. 5,620,675 (1997);
U.S. Pat. No. 5,633,263 (1997);
U.S. Pat. No. 5,650,006 (1997);
U.S. Pat. No. 5,693,679 (1997);
U.S. Pat. No. 5,700,905 (1997);
U.S. Pat. No. 5,708,135 (1998);
U.S. Pat. No. 5,716,596 (1998);
U.S. Pat. No. 5,770,687 (1998);
U.S. Pat. No. 5,776,894 (1998);
U.S. Pat. No. 5,783,170 (1998);
U.S. Pat. No. 5,804,157 (1998);
U.S. Pat. No. 5,811,392 (1998);
U.S. Pat. No. 5,814,298 (1998);
U.S. Pat. No. 5,820,845 (1998);
U.S. Pat. No. 5,830,431 (1998);
U.S. Pat. No. 5,833,942 (1998);
U.S. Pat. No. 5,843,401 (1998);
U.S. Pat. No. 5,843,903 (1998);
U.S. Pat. No. 5,846,934 (1998);
U.S. Pat. No. 5,871,711 (1999);
U.S. Pat. No. 5,874,529 (1999);
U.S. Pat. No. 5,883,293 (1999);
U.S. Pat. No. 5,925,618 (1999);
U.S. Pat. No. 5,929,209 (1999);
U.S. Pat. No. 5,932,189 (1999);
U.S. Pat. No. 5,955,426 (1999);
U.S. Pat. No. 5,965,108 (1999);
U.S. Pat. No. 5,965,694 (1999);
U.S. Pat. No. 5,972,308 (1999);
U.S. Pat. No. 5,981,477 (1999);
U.S. Pat. No. 5,985,241 (1999);
U.S. Pat. No. 6,001,801 (1999);
U.S. Pat. No. 6,001,960 (1999);
U.S. Pat. No. 6,017,509 (2000);
U.S. Pat. No. 6,017,512 (2000);
U.S. Pat. No. 6,020,349 (2000);
U.S. Pat. No. 6,025,372 (2000);
U.S. Pat. No. 6,051,206 (2000);
U.S. Pat. No. 6,051,554 (2000);
U.S. Pat. No. 6,057,338 (2000);
U.S. Pat. No. 6,063,796 (2000);
U.S. Pat. No. 6,083,960 (2000);
U.S. Pat. No. 6,117,880 (2000);
U.S. Pat. No. 6,127,343 (2000);
U.S. Pat. No. 6,183,722 (2001);
U.S. Pat. No. 6,214,316 (2001);
U.S. Pat. No. 6,225,284 (2001);
U.S. Pat. No. 6,241,965 (1999);
U.S. Pat. No. 6,262,229 (2001);
U.S. Pat. No. 6,265,375 (2001);
U.S. Pat. No. 6,316,004 (2001);
U.S. Pat. No. 6,316,414 (2001);
U.S. Pat. No. 6,329,389 (2001);
U.S. Pat. No. 6,352,982 (2002);
U.S. Pat. No. 6,355,613 (2002);
U.S. Pat. No. 6,358,491 (2002);
U.S. Pat. No. 6,387,932 (2002);
U.S. Pat. No. 6,407,059 (2002);
U.S. Pat. No. 6,440,933 (2002);
U.S. Pat. No. 6,465,613 (2002);
U.S. Pat. No. 6,468,974 (2002);
U.S. Pat. No. 6,586,445 (2003);
U.S. Pat. No. 6,602,849 (2003);
U.S. Pat. No. 6,635,647 (2003);
U.S. Pat. No. 6,703,481 (2004);
U.S. Pat. No. 6,727,269 (2004);
U.S. Pat. No. 6,759,415 (2004);
U.S. Pat. No. 6,777,408 (2004);
U.S. Pat. No. 6,818,739 (2004);
U.S. Pat. No. 6,852,725 (2005);
U.S. Pat. No. 6,864,234 (2005);
U.S. Pat. No. 6,872,827 (2005);
U.S. Pat. No. 6,903,074 (2005);
U.S. Pat. No. 6,930,088 (2005);
U.S. Pat. No. 6,943,145 (2005);
U.S. Pat. No. 7,019,004 (2006);
U.S. Pat. No. 7,074,775 (2006);
U.S. Pat. No. 7,084,244 (2006);
U.S. Pat. No. 7,094,753 (2006);
U.S. Pat. No. 7,109,166 (2006);
U.S. Pat. No. 7,115,566 (2006);
U.S. Pat. No. 7,115,634 (2006);
U.S. Pat. No. 7,122,622 (2006);
U.S. Pat. No. 7,144,859 (2006);
U.S. Pat. No. 7,189,856 (2007);
U.S. Pat. No. 7,202,330 (2007);
U.S. Pat. No. 7,220,765 (2007);
U.S. Pat. No. 7,238,695 (2007);
U.S. Pat. No. 7,312,304 (2007);

### U.S. Patent Publication No. 20020052315;

### U.S. Patent Publication No. 20020089576;

### U.S. Patent Publication No. 20020151501;

### U.S. Patent Publication No. 20030114362;

### U.S. Patent Publication No. 20030120072;

### U.S. Patent Publication No. 20030144186;

### U.S. Patent Publication No. 20030153494;

### U.S. Patent Publication No. 20030191134;

### U.S. Patent Publication No. 20030211981;

### U.S. Patent Publication No. 20040006089;

### U.S. Patent Publication No. 20040019069;

### U.S. Patent Publication No. 20040019092;

### U.S. Patent Publication No. 20040023315;

### U.S. Patent Publication No. 20040038970;

### U.S. Patent Publication No. 20040044177;

### U.S. Patent Publication No. 20040082501;

### U.S. Patent Publication No. 20040097418;

### U.S. Patent Publication No. 20040102364;

### U.S. Patent Publication No. 20040110779;

### U.S. Patent Publication No. 20040171530;

### U.S. Patent Publication No. 20040176379;

### U.S. Patent Publication No. 20040181032;

### U.S. Patent Publication No. 20040209798;

### U.S. Patent Publication No. 20040209908;

### U.S. Patent Publication No. 20040242842;

### U.S. Patent Publication No. 20050008573;

### U.S. Patent Publication No. 20050070470;

### U.S. Patent Publication No. 20050090429;

### U.S. Patent Publication No. 20050154039;

### U.S. Patent Publication No. 20050164922;

### U.S. Patent Publication No. 20050226813;

### U.S. Patent Publication No. 20050239796;

### U.S. Patent Publication No. 20050245571;

### U.S. Patent Publication No. 20060063704;

### U.S. Patent Publication No. 20060069017;

### U.S. Patent Publication No. 20060211607;

### U.S. Patent Publication No. 20060223826;

### U.S. Patent Publication No. 20060258572;

### U.S. Patent Publication No. 20060258838;

### U.S. Patent Publication No. 20070032653;

### U.S. Patent Publication No. 20070041902;

### U.S. Patent Publication No. 20070043095;

### U.S. Patent Publication No. 20070129313;

### U.S. Patent Publication No. 20070129422;

### U.S. Patent Publication No. 20070161045;

### U.S. Patent Publication No. 20070259811;

### EP Patent 0 143 307 (1989);

### EP Patent 0 389 180 (1990);

### EP Patent 0 714 911 (2001);

### EP Application 0 030 920 A2 (1980);

### EP Application 0 505 680 A2 (1982);

### EP Application 0 083 305 A2 (1982);

### EP Application 0 203 031 A2 (1986);

### EP Application 0 222 578 A2 (1987);

### EP Application 0 363 589 A2 (1990);

### PCT Publication WO 97.01579 (1997);

### PCT Publication WO 91/18016 (1991);

### PCT Publication WO 91/09056 (1991);

### PCT Publication WO 91/12811 (1990);

### PCT Publication WO 88/05052 (1988);

### U.K. Application GB 2,095,261 (1983);

### French Application FR 2,522,655 (1983).

Exemplary SSTR-1 receptor agonists include, but are not limited to fatty acid salts of Taeg-c(D-Cys-3-Pal-D-Trp-Lys-D-Cys)-Thr(Bzl)-Tyr-NH₂ and Caeg-c(D-Cys-3-Pal-D-Trp-Lys-D-Cys)-Thr(Bzl)-Tyr-NH₂. Exemplary type-2 somatostatin agonists include, but are not limited to fatty acid salts of:
Lanreotide D-2-Nal-c(Cys-Tyr-D-Trp-Lys-Val-Cys)-Thr-NH₂;
Octreotide D-Phe-c(Cys-Phe-D-Trp-Lys-Thr-Cys)-Thr-ol;
[4-(2-hydroxyethyl)]-1-piperazinylacetyl-D-Phe-c(Cys-Tyr-D-Trp-Lys-Abu-Cys)-Thr-NH₂;
c(Tic-Tyr-D-Trp-Lys-Abu-Phe);
4-(2-Hydroxyethyl)-1-piperazine-2-ethanesulfonyl-D-Phe-c(Cys-Tyr-D-Trp-Lys-Abu-Cys)-Thr-NH₂;
[4-(2-hydroxyethyl)]-1-piperazinylacetyl-D-Phe-c(Cys-Tyr-D-Trp-Lys-Abu-Cys)-Thr-NH₂;
D-6-propyl-8beta-ergolinylmethyl-thioacetyl-D-Lys(D-6-propyl-8beta-ergolinyl-methylthioacetyl)-c(Cys-Tyr-D-Trp-Lys-Abu-Cys)-Thr-NH₂;
D-Cpa-c(Cys-Tyr-D-Trp-Lys-Val-Cys)-Thr-NH₂;
D-Phe-Cpa-Tyr-D-Trp-Lys-Val-Phe-Thr-NH₂;
D-Phe-Cpa-Tyr-D-Trp-Lys-Thr-Phe-Thr-NH₂; and
Ac-c(Cys-Lys-Asn-Cpa-Phe-D-Trp-Lys-Thr-Phe-Thr-Ser-D-Cys)-NH₂.

Exemplary fatty acid salts of type-2 somatostatin agonists include, but are not limited to, di-oleate salts of D-2-Nal-c(Cys-Tyr-D-Trp-Lys-Val-Cys)-Thr-NH₂, D-Phe-c(Cys-Phe-D-Trp-Lys-Thr-Cys)-Thr-ol and [4-(2-hydroxyethyl)]-1-piperazinylacetyl-D-Phe-c(Cys-Tyr-D-Trp-Lys-Abu-Cys)-Thr-NH₂

Exemplary type-2 somatostatin antagonists include, but are not limited to fatty acid salts of Cpa-c(D-Cys-3-Pal-D-Trp-Lys-Val-Cys)-Cpa-NH₂; 4FPhe-c(D-Cys-3-Pal-D-Trp-Lys-Tle-Cys)-2-Nal-NH₂; Cpa-c(D-Cys-3 Pal-D-Trp-Lys-Thr-Cys)-2-Nal-NH₂; and Cpa-c(D-Cys-4-Pal-D-Trp-Lys-Thr-Cys)-2-Nal-NH₂.

Exemplary type-5 somatostatin agonists include, but are not limited to fatty acid salts of D-Phe-Phe-Trp-D-Trp-Lys-Thr-Phe-Thr-NH₂ and c(Cys-Phe-Phe-D-Trp-Lys-Thr-Phe-Cys)-NH₂.

B) Angiotensin-converting enzyme (ACE) inhibitors are peptides useful for promoting regeneration of hemopoietic cells in a subject undergoing chemotherapy or radiotherapy.

An exemplary use of an ACE inhibitor comprises the steps of (i) administering to a subject in need thereof a fatty acid salt of AcSDKP(CH₃—CO-Ser-Asp-Lys-Pro-OH) (SEQ ID NO:1) or an agonist thereof, in an amount effective to reduce the proliferation of hemopoietic cells during the chemotherapy or radiotherapy; (ii) administering to the subject an angiotensin-converting enzyme (ACE) inhibitor, in an amount effective to reduce the degradation of said AcSDKP or an agonist thereof by angiotensin-converting enzyme; and (iii) after the chemotherapy or radiotherapy, administering to the subject a hemopoiesis growth factor, in an amount effective to stimulate the proliferation of hemopoietic cells (see W0 97/34627, incorporated herein by reference in its entirety; U.S. Pat. No. 5,739,110, incorporated herein by reference in its entirety). The AcSDKP(CH₃—CO-Ser-Asp-Lys-Pro-OH) (SEQ ID NO:1) or agonist fatty acid salts, the ACE inhibitor and the hemopoiesis growth factor may be administered to the subject in need thereof alone or in any combination via transdermal applications.

C) Bombesin is a tetradecapeptide amide first isolated from the skin of the frog Bombina bombina. It is a potent mitogen for mouse Swiss 3T3 fibroblast cells and also stimulates secretion for guinea pig pancreatic acini. Bombesin-like peptides are produced and secreted by human small cell lung cancer (SCLC) cells and exogenously added bombesin-like peptides can stimulate the growth of human SCLC cells in vitro. Examples of bombesin-like peptides include, but are not limited to, gastrin releasing peptide (GRP) and Neuromedin B (NMB) (see U.S. Pat. No. 5,410,018, incorporated herein by reference in its entirety).

Bombesin analogs are useful for treatment of benign or malignant proliferation of tissue. A number of cancers are known to secrete peptide hormones related to GRP or bombesin. In addition to being a growth factor for SCLC cell lines, bombesin has also been detected in human breast and prostate cancer (Haveman, et al., eds. Recent Results in Cancer Research—Peptide Hormones in Lung Cancer, Springer-Verlag, New York, 1986). Consequently, antagonists to bombesin have been proposed as agents for the treatment of cancers including, but not limited to, colon, prostatic, breast, pancreatic, liver cancer or lung cancer. Bombesin antagonists are also useful for preventing the proliferation of smooth muscle, for suppressing appetite, for stimulating pancreatic secretion and for suppressing a craving for alcohol (see EP Patent No. 0 737 691, incorporated herein by reference in its entirety; U.S. Pat. No. 5,767,236, incorporated herein by reference in its entirety).

Bombesin antagonists may also be administered to a patient in need thereof (i.e., a mammal such as a human) suffering from pulmonary hypertension, to lower either or both systolic or diastolic pulmonary blood pressure (see U.S. Pat. No. 5,650,395, incorporated herein by reference in its entirety).

Exemplary bombesin analogs include, but are not limited to H-pGlu-Gln-Ser-Leu-Gly-Asn-Gln-Trp-Ala-Arg-Gly-His-Phe-Met-NH₂ (SEQ ID NO:2); Gly-Asn-Gln-Trp-Ala-Arg-Gly-His-Phe-Met-NH₂ (SEQ ID NO:3) and H-D-F5-Phe-Gln-Trp-Ala-Val-D-Ala-His-Leu-O—CH₃.

D) Cholecystokinin (CCK) is a hormonal regulator of pancreatic and gastric secretion, of contraction of the gallbladder, and of gut motility. CCK also exists in the brain and may play a role as a central nervous system transmitter (Chang et al., 230 Science 177 (1985)). CCK antagonists are useful in treating and preventing disorders involving CCK, which include but are not limited to, gastrointestinal disorders, for example, involving gastrointestinal motility, e.g., gastroesophageal reflux, gastritis, gastroparesis, biliary dyskenesia, irritable bowel syndrome, acute obstructive cholecystitis, or colitis; or involving colon motility; or involving pancreatic and/or gastric secretion, e.g., acute or chronic pancreatitis, hyperinsulinemia, or Zollinger-Ellison syndrome; antral G cell hyperplasia; or central nervous system disorders, caused by CCK interactions with dopamine, such as neuroleptic disorders, tardive dyskinesia, Parkinson's disease, psychosis or Gilles de la Tourette Syndrome; disorders of appetite regulatory systems; or pain (potentiation of opiate analgesia). The compounds are described as having an antispastic effect on the smoother muscle of the gastroenteric tract, as regulating gastric secretion, and as being protective of gastroenteric mucosa. (see U.S. Pat. No. 5,010,089, incorporated herein by reference in its entirety).

CCK antagonists are useful, alone or in combination with other chemotherapeutic agents, in the treatment of autoproliferative disorders, such as pancreatic cancer or hyperplasia; this activity is believed to occur because of antagonism to the action of cholecystokinin in inducing pancreatic hyperplasia in the presence of known carcinogens, e.g., nitrosamine (see U.S. Pat. No. 5,010,089, incorporated herein by reference in its entirety). Exemplary agonists may be found in U.S. Pat. No. 4,902,708, incorporated herein by reference in its entirety; U.S. Pat. No. 4,814,463, incorporated herein by reference in its entirety, EP Patent No. 0 489 767, incorporated herein by reference in its entirety.

E) Chemokines, such as monocytes chemoattractant protein-1 (MCP-I), play an important role in the recruitment of monocytes and other inflammatory cell types to sites of injury or insult. Macrophage and/or monocyte recruitment plays a role in the morbidity and mortality of a broad spectrum of diseases including autoimmune diseases, granulomatous diseases, infectious diseases, osteoporosis and coronary artery disease. Other inflammatory conditions treatable by chemokine analogs include, but are not limited to, psoriasis, rheumatoid arthritis, inflammatory bowel disease, gouty arthritis, brain inflammation, sepsis, septic shock, acute respiratory distress syndrome, hemorrhagic shock, cardiogenic shock, hypovolemic shock, ischemia and reperfusion injury, multiple sclerosis, pulmonary fibrosis, organ transplant rejection, allergy, chronic obstructive pulmonary disease, asthma and endometriosis. As a result, agents which modulate the activity of chemokines are likely to be useful to prevent and treat a wide range of diseases (see for example, U.S. Pat. No. 5,459,128; incorporated herein by reference in its entirety; WO 09/017,620; incorporated herein by reference in its entirety).

Peptide compounds that inhibit or enhance chemokine-induced activities of other cell types, such as lymphocytes, neutrophils or eosinophils are also target cell types.

Exemplary chemokine analogs include, but are not limited to Ac-c(Cys-Leu-Asp-Pro-Lys-Gln-Lys-Trp-Ile-Gln-Cys)-NH₂ (SEQ ID NO:4), c(Cys-Trp-Ile-Gln-Cys)-NH₂ (SEQ ID NO:5) or c(Gly-Lys-Trp-Ile-Gln-Glu)-NH₂ (SEQ ID NO:6).

F) Recent work has suggested that opioid peptides may be involved in pathological states, including cancer. As shown in U.S. Pat. No. 5,663,295 (incorporated herein by reference in its entirety) multiple opioid receptors are present on numerous tumor cell lines. Opioids have been found to alter cell function and growth (Slotkin et al. Life Sci. 26:861 (1980); Wilson et al. J. Pharmacol. Exp. Ther. 199:368 (1976)), to inhibit the growth of cultured neuroblastoma cells (Zagon et al. Brain Res. Bull. 7:25 (1981)) and to inhibit neuroblastoma tumor growth and prolong survival times in an opioid antagonist sensitive manner in mice with transplanted neuroblastomas, B-16 melanoma cells, MCF-7 breast cancer cells, human lung cancer cells and others (Zagon et al. Life Sci. 28:1095 (1981); Zagon et al. Science 221:671 (1983); and Von Hoff et al. Proc., Am. Assoc. Cancer Res., Abstract 932, p. 236 (1982); Srisuchark et al. Int. J. Immunopharm. 11(5):487 (1989); Minna et al. Proc. Natl. Acad. Sci., 87:3294 (1990); ibid. 89:1169 (1992)).

G) Ghrelin, a recently discovered orexigenic hormone, is produced as a preprohormone that is proteolytically processed to yield a peptide of the following sequence: H-Gly-Ser-Ser-Phe-Leu-Ser-Pro-Glu-His-Gln-Arg-Val-Gln-Gln-Arg-Lys-Glu-Ser-Lys-Lys-Pro-Pro-Ala-Lys-Leu-Gln-Pro-Arg-NH₂ (Kojima, M. et al, Nature, (1999), 402(6762):656-60). Ghrelin is produced by epithelial cells lining the fundus of the stomach and functions to stimulate appetite; its levels increase prior to a meal and decrease thereafter (see WO 08/039,415, incorporated herein by reference in its entirety).

Agonists and antagonists of ghrelin are useful in the treatment of a number of diseases and conditions. For example, agonists of ghrelin, such as (Aib², Glu³(NH-hexyl))hGhrelin(1-28)-NH₂ (SEQ ID NO:7), are useful for inhibiting the effect of glucocorticoids on growth hormone secretion and to counteract the catabolic effects of dexamethasone and other natural glucocorticoids; to ameliorate the catabolic effects of excess glucocorticoids in an individual in need of such treatment; and to allow long term administration of therapeutic doses of glucocorticoids to treat asthma where the ghrelin agonist plays a role in alleviating the catabolic effects of long term administration of therapeutic doses of glucocorticoids. Agonists of ghrelin, such as (Aib², Glu³(NH-hexyl))hGhrelin(1-28)-NH₂ (SEQ ID NO:7), are also useful to ameliorate a reduction in growth, a reduction in growth rate, a reduction in body weight, a reduction in lean body mass, a reduction in IGF-levels and/or a reduction in bone mass (see, for example, WO 07/106,385, incorporated herein by reference in its entirety; see also US Application Publication No. US20050272648A1, incorporated herein by reference in its entirety; WO 07/038,678, incorporated herein by reference in its entirety; WO 08/039,415, incorporated herein by reference in its entirety).

Another exemplary ghrelin/growth hormone releasing hormone agonist is H-Inp-D-Bal-D-Trp-Phe-Apc-NH₂ which is useful is useful for promoting gastric and gastrointestinal motility in a patient (e.g., a mammal such as a human) and as such, is useful for treating conditions benefiting from improved gastric and gastrointestinal motility such as gastroesophageal reflux disease (GERD), IBS, constipation, ileus, emesis, gastroparesis, colonic pseudo-obstruction, and the like (see WO 07/041,278, incorporated herein by reference in its entirety).

H) Growth hormone (GH or somatotropin) is a 191 amino acid peptide which is secreted by the anterior pituitary. Growth hormone itself does not actually promote growth directly but acts by simulating the production of one of the many true growth factors such as the somatomedins produced by the liver. The ultimate effects of growth hormone are widespread however. On a gross level, this hormone affects the skeleton, connective tissue, muscles and viscera. On a molecular level, the metabolic effects of growth hormone and somatomedins include stimulation of nucleic acid and protein synthesis, induction of positive nitrogen balance, stimulation of lipolysis, and a decrease in urea excretion. Inadequate levels of growth hormone in children causes retardation of growth, epiphyseal development and bone age; it also causes retarded development of secondary sexual characteristics. Additional effects of growth hormone deficiency include impaired larynx development, delayed gonadal maturation, and hypoglycemia. These effects can all be reversed with normal levels of growth hormone.

The production of growth hormone is under the control of both releasing and inhibitory influences located in the hypothalamus. The primary releasing influence is growth hormone releasing factor (GRF, also known as GHRH), which is produced primarily in the arcuate nucleus of the hypothalamus and is transported to the pituitary by portal circulation. However, other cells of the body such as pancreatic tumor cells may also produce this hormone. Growth hormone releasing factor (GRF) in humans is a peptide 44 amino acids in length of which the first 29 contain the full biological activity. The discovery of the sequence of GRF has provided a physiologically natural means for treating individuals with growth hormone deficiencies. Substantial efforts have been devoted to development of synthetic analogs of GRF in the hope that such analogs will be more efficient in causing release of growth hormone.

One group of novel GRF hormone analogs are characterized by substitution of the Asn normally located at position 8 in the native molecule with amino acids which are conducive to α-helix formation. The synthetic peptides are useful in any situation in which direct administration of growth hormone would be desired such as for the treatment of growth hormone deficiency-related disorders, such as pituitary dwarfism. Various other metabolic or developmental processes such as wound healing are also affected by growth hormone and may thus benefit by administration of the present GRF analogues (see WO91/16923, incorporated herein by reference in its entirety; see also U.S. Pat. No. 7,456,253, incorporated herein by reference in its entirety).

GRF antagonists, such as GRF peptides with Arg at position 2 in combination with other alterations, particularly at positions 8, 9 and/or 15, are also useful in treatment of conditions caused by excess growth hormone. An example of such a condition is acromegaly, which results in abnormal enlargement of the bones of the face (see WO91/16923, incorporated herein by reference in its entirety).

I) Glucagon-like peptide-1 (7-36) amide (GLP-1) is synthesized in the intestinal L-cells by tissue-specific post-translational processing of the glucagon precursor preproglucagon (Varndell, J. M., et al., J. Histochem Cytochem, 1985:33:1080-6) and is released into the circulation in response to a meal. The plasma concentration of GLP-1 rises from a fasting level of approximately 15 μmol/L to a peak postprandial level of 40 pmol/L (see WO05/058955, incorporated herein by reference in its entirety).

It has been demonstrated that, for a given rise in plasma glucose concentration, the increase in plasma insulin is approximately threefold greater when glucose is administered orally compared with intravenously (Kreymann, B., et al., Lancet 1987: 2, 1300-4). This alimentary enhancement of insulin release, known as the incretin effect, is primarily humoral and GLP-1 is now thought to be the most potent physiological incretin in humans. In addition to the insulinotropic effect, GLP-1 suppresses glucagon secretion, delays gastric emptying (Wettergren A., et al., Dig Dig. Sci 1993:38:665-73) and may enhance peripheral glucose disposal (D'Alessio, D. A. et al., J. Clin Invest 1994:93:2293-6).

In 1994, the therapeutic potential of GLP-1 was suggested following the observation that a single subcutaneous (s/c) dose of GLP-1 could completely normalize postprandial glucose levels in patients with non-insulin-dependent diabetes mellitus (NIDDM) (Gutniak, M. K., et al., Diabetes Care 1994: 17: 1039-44). This effect was thought to be mediated both by increased insulin release and by a reduction in glucagon secretion. Furthermore, an intravenous infusion of GLP-1 has been shown to delay postprandial gastric emptying in patients with NIDDM (Williams, B., et al., J. Clin Endo Metab 1996:81:327-32).

Numerous studies have shown that when given to healthy subjects, GLP-1 potently influences glycemic levels as well as insulin and glucagon concentrations (Orskov, C, Diabetologia 35: 701-711,1992; Hoist, J. J., et al., Potential of GLP-1 in diabetes management in Glucagon iii, Handbook of Experimental Pharmacology, Lefevbre P J, Ed. Berlin, Springer Verlag, 1996, p. 311-326), effects which are glucose dependent (Kreymann, B., et al., Lancet ii: 1300-1304, 1987; Weir, G. C., et al., Diabetes 38: 338-342,1989).

Moreover, it is also effective in patients with diabetes (Gutniak, M., N. Engl'J Med 226: 1316-1322,1992; Nathan, D. M., et al., Diabetes Care 15: 270-276,1992), normalizing blood glucose levels in type 2 diabetic subjects (Nauck, M. A., et al., Diagbetologia 36: 741-744,1993), and improving glycemic control in type 1 patients (Creutzfeldt, W. O., et al., Diabetes Care 19: 580-586,1996), raising the possibility of its use as a therapeutic agent. Additional uses for GLP-1 agonists include, but are not limited to, treatment of Type I diabetes, Type II diabetes, obesity, glucagonomas, secretory disorders of the airway, metabolic disorder, arthritis, osteoporosis, central nervous system disease, restenosis and neurodegenerative diseases (see WO05/058955, incorporated herein by reference in its entirety; see also U.S. Pat. No. 7,368,427, incorporated herein by reference in its entirety; U.S. Pat. No. 7,235,628, incorporated herein by reference in its entirety; U.S. Pat. No. 6,903,186, incorporated herein by reference in its entirety; WO04/074315, incorporated herein by reference in its entirety). Preferred candidate conditions for treatment include Type I and Type II diabetes.

Exemplary GLP-1 agonists include, but are not limited to, (Aib^{8,35})hGLP1(7-36)NH₂(SEQ ID NO:8), (Ser⁸, Aib³⁵) hGLP-1 (7-36) NH₂ (SEQ ID NO:9) and [Aib^{8,35,},Arg³⁴]hGLP1(7-36)-NH₂ (SEQ ID NO:10).

J) Luteinizing hormone-releasing hormone (LHRH) is a neurotransmitter produced by the hypothalamus which stabilizes the secretion of luteinizing hormone (LHRH) and follicle-stimulating hormone (FSH) from the pituitary, which in turn stimulates the synthesis of steroid hormones, such as testosterone, from the gonads. Many LHRH peptide analogs (e.g., agonists and antagonists) are currently sold for the treatment of endometriosis, prostate cancer, precocious puberty and other hormonally mediated disorders (see, for example, U.S. Pat. No. 7,122,628, incorporated herein by reference in its entirety).

Exemplary LHRH agonists include, but are not limited to, pGlu-His-Trp-Ser-Tyr-D-Trp-Leu-Arg-Pro-Gly-NH₂.

K) Melanocortins are a family of regulatory peptides which are formed by post-translational processing of pro-hormone proopiomelanocortin (POMC). POMC is processed into three classes of hormones; the melanocortins, adrenocorticotropin hormone, and various endorphins (e.g. lipotropin) (Cone, et al., Recent Prog. Horm. Res., 51:287-317, (1996); Cone et al., Ann. N.Y. Acad. Sci., 31:342-363, (1993)). Melanocortins have been found in a wide variety of normal human tissues including the brain, adrenal, skin, testis, spleen, kidney, ovary, lung, thyroid, liver, colon, small intestine and pancreas (Tatro, J. B. et al., Endocrinol. 121:1900-1907 (1987); Mountjoy, K. G. et al., Science 257:1248-1251 (1992); Chhajlani, V. et al., FEBS Lett. 309:417-420 (1992); Gantz, I. et al. J. Biol. Chem. 268:8246-8250 (1993) and Gantz, I. et al., J. Biol. Chem. 268:15174-15179 (1993)).

Melanocortin peptides have been shown to exhibit a wide variety of physiological activities including the control of behavior and memory, affecting neurotrophic and antipyretic properties, as well as affecting the modulation of the immune system. Aside from their well known effects on adrenal cortical functions (adrenocorticotropic hormone, ACTH) and on melanocytes (melanocyte stimulating hormone, MSH), melanocortins have also been shown to control the cardiovascular system, analgesia, thermoregulation and the release of other neurohumoral agents including prolactin, luteinizing hormone and biogenic amines (De Wied, D. et al., Methods Achiev. Exp. Pathol. 15:167-199 (1991); De Wied, D. et al., Physiol. Rev. 62:977-1059 (1982); Guber, K. A. et al., Am. J. Physiol. 257:R681-R694 (1989); Walker J. M. et al., Science 210:1247-1249 (1980); Murphy, M. T. et al., Science 221:192-193 (1983); EUerkmann, E. et al., Endocrinol. 130:133-138 (1992) and Versteeg, D. H. G. et al., Life Sci. 38:835-840 (1986)).

Five melanocortin receptors (MC-R) have been characterized to date. These include melanocyte-specific receptor (MC1-R), corticoadrenal-specific ACTH receptor (MC2-R), melacortin-3 (MC3-R), melanocortin-4 (MC4-R) and melanocortin-5 receptor (MC5-R).

There has been great interest in melanocortin (MC-R) receptors as targets for the design of novel therapeutics to treat disorders of body weight such as obesity and cachexia. Both genetic and pharmacological evidence points toward central MC4-R receptors as the principal target (Giraudo, S. Q. et al., Brain Res., 809:302-306 (1998); Farooqi, I. S. et al., NE J. Med., 348:1085-1095 (2003); MacNeil, D. J. et al., Eu. J. Pharm., 44:141-157 (2002); MacNeil, D. J. et al., Eu. J. Pharm., 450:93-109 (2002); Kask, A. et al., NeuroReport, 10:707-711 (1999)). The current progress with receptor-selective agonists and antagonists evidences the therapeutic potential of melanocortin receptor activation, particularly MC4-R.

Melanocortin 4 receptor analogs are also useful to treat a disease or condition selected from the group consisting of general inflammation, inflammatory bowel disease, brain inflammation, sepsis, septic shock, rheumatoid arthritis, gouty arthritis, multiple sclerosis, a metabolic disease or medical condition accompanied by weight gain, obesity, feeding disorders, Prader-Willi Syndrome, a metabolic disease or medical condition accompanied by weight loss, anorexia, bulimia, AIDS wasting, cachexia, cancer cachexia, wasting in frail elderly, skin cancer, endometriosis, uterine bleeding, sexual dysfunction, erectile dysfunction, decreased sexual response in females, organ transplant rejection, ischemia and reperfusion injury, wounding and spinal cord injury, weight loss due to a medical procedure selected from the group consisting of chemotherapy, radiation therapy, temporary or permanent immobilization and dialysis, hemorrhagic shock, cardiogenic shock, hypovolemic shock, cardiovascular disorders, cardiac cachexia, acute respiratory distress syndrome, pulmonary fibrosis, chronic obstructive pulmonary disease, asthma, enhanced immune tolerance, allergies, psoriasis, skin pigmentation depletion, acne, keloid formation, anxiety, depression, memory dysfunction, neuropathic pain, renal cachexia and natriuresis. Other conditions which may be affected by a melanocortin 4 receptor agonist or antagonist include modulation of ovarian weight, placental development, prolactin secretion, FSH secretion, intrauterine fetal growth, parturition, spermatogenesis, thyroxin release, aldosterone synthesis and release, body temperature, blood pressure, heart rate, vascular tone, brain blood flow, blood glucose levels, sebum secretion, pheromone secretion, motivation, learning and behavior, pain perception, neuroprotection, nerve growth, bone metabolism, bone formation and bone development (see WO 07/008,684, incorporated herein by reference in its entirety; WO 07/008,704, incorporated herein by reference in its entirety; WO 08/051,421, incorporated herein by reference in its entirety).

Exemplary melanocortin receptor-4 agonist peptides include, but are not limited to, hydantoin(Arg-Gly)-c(Cys-Glu-His-D-Phe-Arg-Trp-Cys)-NH₂.

L) Pituitary adenylate cyclase activating polypeptide (PACAP) is a member of a super family that already includes several regulatory peptides, e.g., VIP, PHI, PHV, secretin, helodermin, helospectin I and II, glucagon, GIP and GRF (Christophe, J., Biochimica et Biophysica Acta, 1154, 183-199 (1993). This biologically active neuropeptide exists in two amidated forms: PACAP(1-38)-NH₂ (PACAP-38) and PACAP(1-27)-NH₂ (PACAP-27). The deduced amino acid sequence of PACAP-38 in man (Kimura, C., et al., Biochem. Biophys. Res. Commun., 166, 81-89, (1990)) and rat (Ogi, K., et al., Biochem. Biophys. Res. Commun., 173, 1271-1279, (1990)) is identical to that of the isolated ovine PACAP-38.

Agonists of the PACAP receptor are useful in treating cerebrovascular ischemia, male impotence, motor neuron disease, neuropathy, pain, depression, anxiety disorders, brain trauma, memory impairments, dementia, cognitive disorder, central nervous system diseases (such as Parkinson's disease, Alzheimer's disease), migraine, neurodegenerative diseases, ischemic heart disease, myocardial infarction, fibrosis, restenosis, diabetes mellitus, muscle disease, gastric ulcer, stroke, atherosclerosis, hypertension, septic shock, thrombosis, retina disease, cardiovascular disease, renal failure and cardiac failure and the prevention of neuronal cell death in a mammal (see for example, U.S. Pat. No. 6,242,563, incorporated herein by reference in its entirety).

M) Parathyroid hormone (PTH) is the principal physiological regulator of calcium levels in the blood (Chorev, M., Rosenblatt, M., 1994, Bilezikian, J. P., Marcus, R., Levine, M., (eds) The Parathyroids: Basic and Clinical Concepts. Raven Press, New York, pp 139-156; Juppner, H., et al., 1991, Science, 254:1024-1026; Martin, T. J., et al., 1991, Crit. Rev. Biochem. Mol. Biol. 26:377-395). PTH-related protein (PTHrP) was originally identified as the agent responsible for the paraneoplastic syndrome of humoral hypercalcemia of malignancy (Suva, L. J., et al., 1987, Science, 237:893-896 and Orloff, J. J., et al., 1994, Endocrinol. Rev. 15:40-60).

PTH and PTHrP are products of distinct yet evolutionarily-related genes which show sequence similarities only in the N-terminal 13 amino acids, eight of which are identical (Abou-Samra A B, et al., 1992, Proc. Natl. Sci. Acad. USA, 89:2732-2736). The expression pattern and physiological role of these two molecules however, are remarkably different.

Both PTH and PTHrP share a common G protein-coupled receptor termed the PTH/PTHrP receptor. The PTH2 receptor is localized predominantly in the brain and pancreas in contrast to PTH/PTHrP receptor which is primarily localized in bone and the kidney, the principal target tissue for PTH action. The PTH/PTHrP receptor is a member of a subfamily of G protein-coupled receptor superfamily, which includes the receptors for glucagon, growth hormone-releasing hormone (GHRH), vasoactive intestinal peptide (VIP), glucagon-like peptide 1 (GLP-1), gastric inhibitory polypeptide (GIP), secretin, pituitary adenylate cyclase-activating polypeptide (PACAP), calcitonin, and corticotropin-releasing factor (CRF) (Juppner, H., et al., 1988, J. Biol. Chem., 263:1071-1078; Shigeno, C., et al., 1988, J. Biol. Chem., 263:18369-18377; Behar, V., et al., 1996, Endocrinology, 137:2748-2757; Gardella, T. J., et al., 1996, The J. Biol. Chem., 271:19888-19893; Behar, V., et al., 1996, Endocrinology, 137:4217-4224; and Usdin, T. B., et al., 1997, Endocrinology, 138:831-834; Segre, G., et al., 1993, Trends Endocrinol.).

Amongst the seven transmembrane G protein-coupled receptors, the PTH2 receptor is most similar in sequence to the PTH/PTHrP receptor (51% amino acid sequence identity). Interestingly, PTH2 receptor mRNA is not detected in bone or osteosarcoma cell lines, but is expressed in a number of tissues including the exocrine pancreas, lung, heart, vasculature and epididymis, and is most abundant in the brain (Usdin, T. B., et al., 1996, Endocrinology, 137:4285-4297). Unlike the PTH/PTHrP receptor, which binds and is activated by both PTH-(1-34) and PTHrP-(1-34), the PTH2 receptor binds and is activated only by PTH-(1-34). PTHrP (7-34) was found to recognize PTH2 receptor and weakly activate it.

PTH and PTHrP analogues that selectively bind to PTH2 receptors are useful in treating abnormal CNS functions; abnormal pancreatic functions; divergence from normal mineral metabolism and homeostasis; male infertility; regulation of abnormal blood pressure; and hypothalmic disease. PTHrP and certain analogs are also known to be useful to improve bone mass and quality in the treatment of osteoporosis and related disorders. (see also U.S. Pat. No. 7,531,621, incorporated herein by reference in its entirety; U.S. Pat. No. 6,921,750, incorporated herein by reference in its entirety; U.S. Pat. No. 5,955,574, incorporated herein by reference in its entirety; U.S. Pat. No. 6,544,949, incorporated herein by reference in its entirety; 5,723,577, incorporated herein by reference in its entirety; 5,696,095, incorporated herein by reference in its entirety; WO 08/063,279, incorporated herein by reference in its entirety; International Patent Application No. PCT/US09/002,868, incorporated herein by reference in its entirety; U.S. patent application Ser. No. 12/311,418, incorporated herein by reference in its entirety; U.S. patent application Ser. No. 12/151,975, incorporated herein by reference in its entirety).

Exemplary parathyroid hormone releasing hormone peptide agonists include, but are not limited to, Glu^{22,25,}Leu^{23,28,31,}Aib^{29,}Lys^{26,30}]hPTHrP(1-34)-NH₂ (SEQ ID NO:11).

N) Peptide YY (PYY) is a 36-residue peptide amide isolated originally from porcine intestine. It is localized in the endocrine cells of the gastrointestinal tract and pancreas (Tatemoto et al. Proc. Natl. Acad. Sci. 79: 2514, 1982). PYY shares a number of central and peripheral regulatory roles with its homologous peptide Neuropeptide Y (NPY) which was originally isolated from porcine brain (Tatemoto, Proc. Natl. Acad. Sci. 79: 5485, 1982).

PYY is localized in intestinal cells; NPY, in contrast, is present in the submucous and myenteric neurons which innervate the mucosal and smooth muscle layers, respectively (Ekblad et al. Neuroscience 20: 169, 1987). Both PYY and NPY are believed to inhibit gut motility and blood flow (Laburthe, Trends Endocrinol. Metab. 1: 168, 1990), and they are also thought to attenuate basal (Cox et al. Br. J. Pharmacol. 101: 247, 1990; Cox et al. J. Physio. 398: 65, 1988; Cox et al. Peptides 12: 323, 1991; Friel et al. Br. J. Pharmacol. 88: 425, 1986) and secretagogue-induced intestinal secretion in rats (Lundberg et al. Proc. Nail. Acad. Sci. USA 79: 4471, 1982; Playford et al. Lancet 335: 1555, 1990) and humans (Playford et al., supra), as well as stimulate net absorption (MacFadyen et al. Neuropeptides 7: 219, 1986). Elevated plasma PYY levels have been reported in individuals suffering from several conditions that cause diarrhea (Adrian et al. Gastroenterology 89: 1070, 1985).

Taken together, these observations suggest that PYY and NPY are released into the circulation after a meal (Adrian et al. Gastroenterology 89: 1070, 1985; Balasubramaniam et al. Neuropeptides 14: 209, 1989) and thus may play a physiological role in regulating intestinal secretion and absorption to serve as natural inhibitors of diarrhea. PYY and NYY agonists are thus contemplated to modulate nutrient availability in a patient for treating metabolic disorders which affect nutrient availability such as, but not limited to, obesity, diabetes, including but not limited to type 2 or non-insulin dependent diabetes, eating disorders, insulin-resistance syndrome and cardiovascular disease. Additional conditions or diseases amenable to treatment by agonists of PYY or NPY include, but are not limited to, hypertension, dyslipidemia, gall stones, osteoarthritis and cancers, conditions characterized by weight loss such as anorexia, bulimia, cancer cachexia, AIDS, wasting, cachexia and wasting in frail elderly as well as weight loss resulting from chemotherapy, radiation therapy, temporary or permanent immobilization or dialysis (see WO04/066966, incorporated herein by reference in its entirety).

Exemplary peptide Y or neuropeptide γ agonists include, but are not limited to, [camptothecin-rvGly-Suc-Tyr¹, Nle¹⁷, Pro³⁴]hNPY(1-36)-NH₂ (SEQ ID NO:12), [camptothecin-rvD/LAsp-Suc-Tyr¹, Nle¹⁷, A6c³¹, 4Hyp³⁴]hNPY(1-36)-NH₂ and [camptothecin-rvD/L-Asp-Suc-Tyr¹, Nle¹⁷, A6c³¹, 4Hyp³⁴]hNPY(1-36)-NH₂

O) ADROPIN c(Cys-His-Ser-Arg-Ser-Ala-Asp-Val-Asp-Ser-Leu-Ser-Glu-Ser-Ser-Pro-Asn-Ser-Ser-Pro-Gly-Pro-Cys)-Pro-Glu-Lys-Ala-Pro-Pro-Pro-Gln-Lys-Pro-Ser-His-Glu-Gly-Ser-Tyr-Leu-Leu-Gln-Pro) (SEQ ID NO:13) is a secreted peptide that is encoded by a gene highly expressed in the liver and central nervous system that is involved in regulating energy homeostasis and lipid metabolism in response to dietary nutrient composition. ADROPIN [derived from the Latin root “aduro” (to set fire to) and “pinquis” (fats or oils)] is encoded by the “Energy Homeostasis Associated” transcript (gene symbol: Enho (previously referred to as Swir1); see WO 2007/019426 incorporated herein in its entirety; Kumar KG,@ et al., Adropin is a secreted peptide involved in energy homeostasis and lipid metabolism, 2008, submitted to Cell Metabolism, incorporated herein in its entirety).

Obesity and insulin resistance are two common disorders of energy homeostasis which result from an organism's failure to balance and adapt energy homeostasis, particularly under conditions of abundant calorie-dense food and reduced physical activity-based energy expenditure (Hill, J. A., Endocrine Reviews, 2006, 27:750-761).

In addition to obesity and insulin resistance, imbalances in energy homeostasis may also contribute to increased lipid metabolism, diabetes, particularly type-2 diabetes, non-alcoholic fatty liver disease and Syndrome X and associated complications such as hypertension, blood glucose and triglyceride levels and the like.

P) Urotensin-II (U-II) is a cyclic neuropeptide with potent cardiovascular effects. Sequence analysis of various U-II peptides from different species has revealed that while the N-terminal region is highly variable, the C-terminal cyclic region of U-II is strongly conserved. Indeed, this cyclic region, which is responsible for the biological activity of U-II, is fully conserved from fish to humans (Coulouran, et al., Proc. Natl. Acad. Sci. USA (physiology), 95:15803-15808 (1998)). The cyclic region of U-II includes six amino acid residues Cys-Phe-Trp-Lys-Tyr-Cys.

In fish, U-II peptides have been shown to exhibit several activities including general smooth muscle contracting activity (Davenport, A., and Maguire, J., Trends in Pharmacological Sciences, 21:80-82 (2000); Bern, H. A., et al., Recent Prog. Horm. Res., 45:533-552 (1995)). Human U-II is found within both vascular and cardiac tissue (including coronary atheroma) and effectively constricts isolated arteries from non-human primates (Ames, H., et al., Nature, 401:282-286, 1999). The vasoconstrictive potency of U-II is substantially greater than that of endothelin-1, making human U-II one of most potent mammalian vasoconstrictors currently known.

Since human U-II-like immunoreactivity is found within cardiac and vascular tissue (including coronary atheroma), U-II is believed to influence cardiovascular homeostasis and pathology (e.g., ischemic heart disease and congestive heart failure). Furthermore, the detection of U-II immunoreactivity within spinal cord and endocrine tissues suggests that U-II may have additional activities, including modulation of central nervous system and endocrine function in humans (Ames, H., et al., supra). Excess or reduced expression of U-II activity has been found to correlate with a number of conditions including but not limited to, ischaemic heart disease, congestive heart failure, portal hypertension, variceal bleeding, hypotension, angina pectoris, myocardial infarction, ulcers, anxiety, schizophrenia, manic depression, delirium, dementia, mental retardation, and dyskinesias (see also U.S. Pat. No. 7,241,737, incorporated herein by reference in its entirety).

Exemplary urotensin II agonists include, but are not limited to, Asp-c[Cys-Phe-Trp-Lys-Tyr-Cys]-Val-OH (SEQ ID NO:14) and exemplary antagonists include, but are not limited to, Cpa-c[D-Cys-Pal-D-Trp-Lys-Val-Cys]-Cpa-NH₂ and Cpa-c[D-Cys-Phe-Trp-Lys-Thr-Cys]-Val-NH₂.

Q) Glucose-dependent insulinotropic polypeptide (GIP, also known as gastric inhibitory polypeptide) is a 42-residue peptide secreted by enteroendocrine K-cells of the small intestine into the bloodstream in response to oral nutrient ingestion. GIP inhibits the secretion of gastric acid and it has been shown to be a potent stimulant for the secretion of insulin from pancreatic beta cells after oral glucose ingestion (the incretin effect; Creutzfeldt, W., et al., 1979, Diabetologia, 16:75-85).

Insulin release induced by the ingestion of glucose and other nutrients results from both hormonal and neuronal factors (Creutzfeldt, W., et al., 1985, Diabetologia, 28:565-573). Several gastrointestinal regulatory peptides have been proposed as incretins and among these candidates, only GIP and glucagon-like peptide 1 (GLP-1) appear to fulfill the requirements to be considered physiological stimulants of postprandial insulin release (Nauck, et al., 1989, J. Clin. Endocrinol. Metab., 69:654-662). It has been shown that the combined effects of GIP and GLP-1 are sufficient to explain the full incretin effect of the enteroinsular axis (Fehmann, H. C., et al., 1989, FEBS Lett., 252:109-112).

The known and potential uses of GIP are varied and multitudinous and may be summarized as follows: treating a disease selected from the group consisting of type 1 diabetes, type 2 diabetes (Visboll, T., 2004, Dan.

Med. Bull., 51:364-70), insulin resistance (WO 2005/082928), obesity (Green, B. D., et al., 2004, Current Pharmaceutical Design, 10:3651-3662), metabolic disorder (Gault, V. A., et al., 2003, Biochem. Biophys. Res. Commun., 308:207-213), central nervous system disease, neurodegenerative disease, congestive heart failure, hypoglycemia, and disorders wherein the reduction of food intake and weight loss are desired. In pancreatic islets, GIP not only acutely enhances insulin secretion, but it also stimulates insulin production through enhancement of proinsulin transcription and translation (Wang, et al., 1996, Mol. Cell. Endocrinol., 116:81-87) and enhances the growth and survival of pancreatic beta cells (Trumper, et al., 2003, Diabetes, 52:741-750). In addition to effects on the pancreas to enhance insulin secretion, GIP also exhibits direct effects on insulin target tissues to lower plasma glucose: enhancement of glucose uptake in adipose (Eckel, et al., 1979, Diabetes, 28:1141-1142) and muscle (O'Harte, et al., 1998, J. Endocrinol., 156:237-243), and inhibition of hepatic glucose production (Elahi, D., et al., 1986, Can. J. Physiol. Pharmacol., 65:A18).

GIP receptor antagonists inhibit, block or reduce glucose absorption from the intestine of an animal. Thus therapeutic compositions containing GIP antagonists may be used in patients with non-insulin dependent diabetes mellitus to improve tolerance to oral glucose in mammals, such as humans, to prevent, inhibit or reduce obesity by inhibiting, blocking or reducing glucose absorption from the intestine of the mammal.

GIP analogues useful for the practice of the instant invention may be found in PCT publication WO 00/58360 (incorporated herein by reference in its entirety) which discloses peptidyl analogues of GIP which stimulate the release of insulin; PCT publication WO 98/24464 (incorporated herein by reference in its entirety) which discloses an antagonist of GIP for treating non-insulin dependent diabetes mellitus and for improving glucose tolerance in a non-insulin dependent diabetes mellitus patient; PCT publication WO 03/082898 (incorporated herein by reference in its entirety) which discloses C-terminal truncated fragments and N-terminal modified analogues of GIP alleged to be useful in treating GIP-receptor mediated conditions, such as non-insulin dependent diabetes mellitus and obesity. See also International Application Serial No. PCT/US09/04552 (incorporated herein by reference in its entirety); International Application Serial No. PCT/US09/04543 (incorporated herein by reference in its entirety), International Application Serial No. PCT/US09/04550, (incorporated herein by reference in its entirety), International Application Serial No. PCT/US09/04559, (incorporated herein by reference in its entirety) and International Application Serial No. PCT/US09/04545, (incorporated herein by reference in its entirety).

Exemplary GIP analogs include, but are not limited to,

(D-Ala², A5c¹¹, His⁴³)hGIP(1-43)-OH;
(D-Ala², A5c^{11,41})hGIP(1-42)-OH;
(SEQ ID NO: 15)
(Gly², A5c¹¹, Nle¹⁴, His⁴³)hGIP(1-43)-OH;
(D-Ala², A5c¹¹, Nle¹⁴, His⁴³)hGIP(1-43)-OH;
(SEQ ID NO: 16)
(A5c^{11,41})hGIP(1-42)-OH;
(SEQ ID NO: 17)
(A5c^{11,40})hGIP(1-42)-OH;
(SEQ ID NO: 18)
(A5c¹¹, His⁴³)hGIP(1-43)-OH;
(SEQ ID NO: 19)
(A5c¹¹, Asn⁴³)hGIP(1-43)-OH;
(SEQ ID NO: 20)
(Aib¹³, Asp⁴³)hGIP(1-43)-NH₂;
(SEQ ID NO: 21)
(Aib¹³, Nle¹⁴, A5c⁴⁰)hGIP(1-42)-OH;
(SEQ ID NO: 22)
(Aib¹³, A5c⁴⁰)hGIP(1-42)-OH;
(SEQ ID NO: 23)
(A5c^{11,14}, His⁴³)hGIP(1-43)-OH;
(SEQ ID NO: 24)
(3Cl-Tyr¹, Aib², A5c¹¹, Nle¹⁴)hGIP(1-42)-OH;
(SEQ ID NO: 25)
(3MeO-Tyr¹, Aib², A5c^{11,41})hGIP(1-42)-OH;
SEQ ID NO: 26
(Aib², A5c¹¹, Nle¹⁴)hGIP(1-42)-OH;
SEQ ID NO: 27
[A5c¹¹, Lys⁴³(N—C(O)—(CH₂)₁₄—CH₃)]hGIP(1-43)-OH;
[3Cl-Tyr¹, D-Ala², A5c¹¹, Nle¹⁴, Lys⁴³(N—C(O)—(CH₂)₁₀-CH₃)]hGIP(1-43)-OH;
SEQ ID NO: 28
[3Cl-Tyr¹, Aib², A5c¹¹, Nle¹⁴, Cys⁴³(succinimide-N-30K PEG)]hGIP(1-43)-NH₂;
SEQ ID NO: 29
[3Cl-Tyr¹, Aib², A5c¹¹, Nle¹⁴, Cys⁴³(succinimide-N—(CH₂)₂—C(O)NH—(CH₂)₃—O—CH₂—
CH(20K PEG)-CH₂-20K PEG)]hGIP(1-43)-NH₂;
SEQ ID NO: 30
(Aib², A6c⁷)hGIP(1-30)-NH₂,
SEQ ID NO: 31
(Aib^{2,13}, Nle¹⁴)hGIP(1-42)-OH;
(D-Ala², A5c^{11,14})hGIP(1-30)-NH₂;
SEQ ID NO: 32
[A5c¹¹, Lys⁴³(N—C(O)—(CH₂)₁₀—CH₃)]hGIP(1-43)-OH;
SEQ ID NO: 33
(A5c^{11,41}, Cys⁴³)hGIP(1-43)-NH₂;
SEQ ID NO: 34
(hTyr¹, Aib², A5c¹¹, His⁴³)hGIP(1-43)-OH;
[3Cl-Tyr¹, D-Ala², A5c^{11,14}, Lys⁴³(N—C(O)—(CH₂)₁₀—CH₃)]hGIP(1-43)-OH;
[3Cl-Tyr¹, D-Ala², A5c¹¹, Nle¹⁴, Cys⁴³(succinimide)]hGIP(1-43)-OH;
SEQ ID NO: 35
(Ac-A6c⁷, Gln⁴³)hGIP(7-43)-OH;
SEQ ID NO: 36
(4Hppa², Aib¹³)hGIP(2-42)-OH;
SEQ ID NO: 37
(3Cl-Tyr¹, Aib², A5c¹¹, Nle¹⁴, Cys⁴³)hGIP(1-43)-OH;
SEQ ID NO: 38
[3Cl-Tyr¹, Aib², A5c¹¹, Nle¹⁴, Cys⁴³(succinimide-N-20K PEG)]hGIP(1-43)-NH₂,
SEQ ID NO: 41
[A5c^{11,41}, Cys⁴³(succinimide-N-30K PEG)]hGIP(1-43)-NH₂

R) Insulin-like growth factor-1 (IGF-1) is a 70-amino-acid polypeptide hormone having insulin-like and mitogenic growth biological activities. This hormone enhances growth of cells in a variety of tissues including musculoskeletal systems, liver, kidney, intestines, nervous system tissues, heart and lung.

While IGF-1 is present in a wide variety of body tissues, it is normally found in an inactive form in which it is bound to an IGF binding protein (IGFBP). Disruption of IGF-1 action may contribute to a number of physiological disorders including neurodegenerative disorders such as motor neuron disease (i.e., amyotrophic lateral sclerosis (ALS)), muscular dystrophy and multiple sclerosis, cartilage disorders such as osteoarthritis, bone diseases such as osteoporosis, inflammatory disorders such as rheumatoid arthritis, ischemic injuries to organs such as to the heart, brain, or liver, and so forth.

As is well known to those skilled in the art, the known and potential uses of IGF-1 are varied and multitudinous. For example, a number of studies report on the use of IGF-1 as a potential therapeutic agent for treatment of neurodegenerative conditions. See, e.g., Kanje et al., Brain Res., 486:396-398 (1989); Hantai et al., J. Neurol. Sci., 129:122-126 (1995); Contreras et al., Pharmac. Exp. Therap., 274:1443-1499 (1995); Di Giulio et al., Society for Neuroscience, 22:1960 (1996); Di Giulio et al., Society for Neuroscience, 23:894 (1997); Hsu et al., Biochem. Mol. Med., 60(2):142-148 (1997); Gorio et al., Neuroscience, 82:1029-1037 (1998). IGF-1 therapy has been indicated in numerous neurological conditions, including ALS, stroke, epilepsy, Parkinson's disease, Alzheimer's disease, acute traumatic injury and other disorders associated with trauma, aging, disease, or injury. See, e.g., U.S. Pat. Nos. 5,093,137; 5,652,214; 5,703,045; International Publication Nos. WO 90/1483 and WO 93/02695.

Use of IGF-1 therapy for a variety of other conditions has been referred to in a number of publications. See, e.g., Schalch et al., “Modern Concepts of Insulin-Like Growth Factors,” ed. Spencer (Elsevier, N.Y.), pp. 705-714 (1991); Clemmons and Underwood, J. Clin. Endocrinol. Metab., 79(1):4-6 (1994); and Langford et al., Eur. J. Clin. Invest., 23(9):503-516 (1993) (referring to, e.g., insulin-resistant states and diabetes); and O'Shea et al., Am. J. Physiol., 264:F917-F922 (1993) (referring to, e.g., reduced renal function). Also see U.S. Pat. No. 7,258,864 (referring to short stature); U.S. Pat. Nos. 5,110,604 and 5,427,778 (referring to, e.g., wound healing); U.S. Pat. No. 5,126,324 (referring to, e.g., cardiac disorders and growth retardation); U.S. Pat. No. 5,368,858 (referring to, e.g., defects or lesions in cartilage); U.S. Pat. Nos. 5,543,441 and 5,550,188 (referring to, e.g., tissue augmentation); U.S. Pat. No. 5,686,425 (referring to, e.g., scar tissue, localized muscular dysfunction, and urinary incontinence); and U.S. Pat. No. 5,656,598 (referring to, e.g., bone growth). Also see International Publication Nos. WO 91/12018 (referring to, e.g., intestinal disorders); WO 92/09301 and WO 92/14480 (referring to, e.g., wound healing); WO 93/08828 (referring to, e.g., neuronal damage associated with ischemia, hypoxia, or neurodegeneration); WO 94/16722 (referring to, e.g., insulin resistance); WO 96/02565A1 (referring to, e.g., IGF/IGFBP complex for promoting bone formation and for regulating bone remodeling); U.S. Patent Application Publication No. 2003/0100505 (referring to, e.g., osteoporosis); and U.S. Patent Application Publication No. 2005/0043240 (referring to obesity). See also U.S. Patent Application No. 61/271,549, (incorporated herein by reference in its entirety), U.S. Patent Application No. 61/271,513, (incorporated herein by reference in its entirety) and U.S. Patent Application No. 61/271,551, (incorporated herein by reference in its entirety).

S) Peptides which promote apoptosis or programmed cell death are also suitable for use in the practice of the instant invention. The proliferation rate of a cell population reflects a balance between cell division, cell cycle arrest, differentiation and programmed cell death or apoptosis (Rudin, C. M. and Thompson, Annu. Rev. Med., 48: 267-81, 1997). The regulation of these processes is central to development and tissue homeostasis, whereas dysregulation may lead to overt pathological outcomes, most notably cancer and neurodegenerative disorders (Spengler, D., et al., EMBO J., 16: 2814-2825, 1997).

Apoptosis comprises an intrinsic cellular defense against tumorigenesis which, when suppressed, may contribute to the development of malignancies (Reed, J. C., Cancer J. Sci. Am., 4 Suppl 1: S8-14, 1998). The Bcl-2 oncogene product functions as a potent suppressor of apoptosis under diverse conditions (Kroemer, G. (published erratum appears in Nat Med 1997 August; 3 (8): 934), Nat. Med., 3: 614-620, 1997). Bcl-2 inhibits apoptosis induced by a wide variety of stimuli and is found to be over-expressed in many types of human tumors. Protein-protein interaction between members of the Bcl-2 family of proteins (Antonawich, F. J., et al., J. Cereb. Blood Flow Metab., 18: 882886, 1998), and other death-promoting proteins such as Bad, Bak, Bax, Bip1, Bik and Bcl-xS (Boyd, J. M., et al., Oncogene, 11: 1921-1928, 1995; Jurgensmeier, J. M., et al., Proc. Natl. Acad. Sci. U.S.A., 95: 4997-5002, 1998; and Chittenden, T., et al., Nature, 374: 733-736, 1995) are believed to be key events in the regulation of apoptosis.

One domain in Bak, termed BH-3, was identified to be both necessary and sufficient for cytotoxic activity and binding to Bcl-xL (Chittenden, T., et al., EMBO J., 14: 5589-5596,1995). Sequences similar to this domain were identified in Bax and Bip1, two other proteins that promote apoptosis and interact with Bcl-xL, and were likewise critical for their capacity to kill cells and bind Bcl-xL. Thus, the BH3 domains of pro-apoptotic proteins are sufficient to trigger apoptosis accompanied by the release of cytochrome C from mitochondria and caspase activation. Synthetic peptides which reproduce the effect of proapoptotic BH3 domains suggests that such peptides may be useful in the diagnosis and treatment of proliferative disease and may provide the basis for engineering reagents to control the initiation of apoptosis (see WO 01/00670, incorporated herein by reference in its entirety).

Exemplary apoptotic control genes include peptides containing a BH-3 domain such as, but not limited to Ac-Leu-Ser-Glu-Cys-Leu-Lys-Arg-Ile-Gly-Asp-Glu-Leu-Asp-Ser-Asn-NH₂ (SEQ ID NO: 39) and Ac-Leu-Ser-Glu-Ser-Leu-Lys-Arg-Ile-Gly-Asp-Glu-Leu-Asp-Ser-Asn-NH₂(SEQ ID NO: 40).

T) Peptides that act as biological receptor ligands joined with a cytotoxic moiety are also suitable for practice the instant invention. Most cytotoxic drugs exhibit undesirable toxic side effects due to their lack of selective action toward the tissues or cells requiring therapeutic effect. Various approaches have been pursued to achieve the selective delivery of cytotoxic agents to a target cell type. Using biological receptor ligands as carriers of drugs to target these drugs to the cells of interest can reduce toxic side-effects and greatly improve the efficiency drug delivery.

For example, International Patent Publication No. WO97/19954 discloses conjugates of an anthracycline cytotoxic agent such as doxorubicin with a peptide hormone such as LHRH, bombesin or somatostatin. The cytotoxic agent is covalently attached to the peptide via a linker of formula —C(O)—(CH₂)ₙ—C(O)—, n=0-7.

Similarly, European Patent Application No. EP1118336 discloses conjugates of somatostatin analogs, e.g., octreotide, lanreotide, and vapreotide, and a cytotoxic drug, such as paclitaxel, doxorubicin, or camptothecin, through a spacer, wherein the spacer is also indicated to have the structure: —C(O)—(CH₂)ₙ—C(O)—, n=0-7.

U.S. Patent Application Publication No. 2002/0115596 discloses conjugates of cytotoxic agents and oligopeptides in which the amino acid sequences of the peptides are indicated to be cleaved preferentially by free prostate specific antigen. Such conjugates are said to be useful for the treatment of prostate cancer and benign prostatic hyperplasia.

U.S. Patent Application Publication No. 2003/0064984 discloses conjugates of cytotoxic analogs of CC-1065 and the duocarmycins with cleavable linker arms and a targeting agent such as an antibody or a peptide. The cytotoxic analogs are indicated to be released upon cleavage of the linker.

International Patent Publication No. WO 04/093807 (incorporated herein by reference in its entirety) discloses a number of preferred peptide cytotoxic peptide conjugates for use with the instant invention.

Exemplary peptide-cytotoxic conjugates include, but are not limited to, conjugates of anthracycline cytotoxic agents and fatty acid salts of peptide hormones such as LHRH, bombesin or somatostatin.
Camptothecin-Gly-glutaryl-AEPA-Lys-D-Tyr-D-Tyr-c(Cys-Tyr-D-Trp-Lys-Abu-Cys)-Thr-NH₂;
Camptothecin-Gly-glutaryl-Lys-D-Tyr-DTyr-c(Cys-Tyr-D-Trp-Lys-Abu-Cys)-Thr-NH₂;
Camptothecin-20-glycinyl-succinoyl-D-Phe-c(Cys-Tyr-D-Trp-Lys-Abu-Cys)-Thr-NH₂;
Doxorubicin-Gly-Suc-(Doc)₄-Aepa-D-Phe-c(Cys-Tyr-D-Trp-Lys-Abu-Cys)-Thr-NH₂;
Camptothecin-Gly-Glut-(Doc)₄-Lys-D-Tyr-DTyr-cyclo(Cys-Tyr-D-Trp-Lys-Abu-Cys)-Thr-NH2;
Camptothecin-CONH—(CH₂)₂—S—S—(CH₂)—CO-(Doc)₄-Lys-D-Tyr-DTyr-c(Cys-Tyr-D-Trp-Lys-Abu-Cys)Thr-NH₂;
Camptothecin-rvD-Asp-suc-(Doc)₄-Aepa-D-Phe-c(Cys-Tyr-D-Trp-Lys-Abu-Cys)-Thr-NH₂;

Camptothecin-rvCha-suc-(Doc)₄-Aepa-D-Phe-c(Cys-Tyr-D-Trp-Lys-Abu-Cys)-Thr-NH₂;
Camptothecin-rvAbu-suc-(Doc)₄-Aepa-D-Phe-c(Cys-Tyr-D-Trp-Lys-Abu-Cys)-Thr-NH₂;
Camptothecin-rvVal-suc-(Doc)₄-Aepa-D-Phe-c(Cys-Tyr-D-Trp-Lys-Abu-Cys)-Thr-NH₂;
Camptothecin-C(O)—N((CH₂)₂NHCH₃)-β-Ala-(Doc)₄-Aepa-D-Phe-c(Cys-Tyr-D-Trp-Lys-Abu-Cys)-Thr-NH₂;
CamptothecinSN38-rvGly-Suc-(Doc)₄-Aepa-D-Phe-c(Cys-Tyr-D-Trp-Lys-Abu-Cys)-Thr-NH₂;
Camptothecin-rvD-Asp-Suc-(Peg₃)₃-Aepa-D-Phe-c(Cys-Tyr-D-Trp-Lys-Abu-Cys)-Thr-NH₂; and
Paclitaxel-glutaryl-D-Phe-c(Cys-Phe-D-Trp-Lys-Thr-Cys)-Thr-ol.

Note that for all peptides described herein, each amino acid residue represents the structure of —NH—C(R)H—CO—, in which R is the side chain (e.g., CH₃ for Ala). Lines between amino acid residues represent peptide bonds which join the amino acids. Also, where the amino acid residue is optically active, it is the L-form configuration that is intended unless D-form is expressly designated. For clarity, disulfide bonds (e.g., disulfide bridge) which exist between two free thiols of Cys residues are not shown. Abbreviations of the common amino acids are in accordance with IUPAC-IUB recommendations.

Some of the compounds of the instant invention can have at least one asymmetric center. Additional asymmetric centers may be present on the molecule depending upon the nature of the various substituents on the molecule. Each such asymmetric center will produce two optical isomers and it is intended that all such optical isomers, as separated, pure or partially purified optical isomers, racemic mixtures or diastereomeric mixtures thereof, are included within the scope of the instant invention.

The compounds of the instant invention generally can be isolated in the form of their pharmaceutically acceptable acid addition salts, such as the salts derived from using inorganic and organic acids. Examples of such acids are hydrochloric, nitric, sulfuric, phosphoric, formic, acetic, trifluoroacetic, propionic, maleic, succinic, D-tartaric, L-tartaric, malonic, methane sulfonic and the like. In addition, certain compounds containing an acidic function such as a carboxy can be isolated in the form of their inorganic salt in which the counter-ion can be selected from sodium, potassium, lithium, calcium, magnesium and the like, as well as from organic bases.

The pharmaceutically acceptable salts can be formed by taking about 1 equivalent of an SSTR-2 agonist, e.g., c[Tic-Tyr-D-Trp-Lys-Abu-Phe], and contacting it with about 1 equivalent or more of the appropriate corresponding acid of the salt which is desired. Work-up and isolation of the resulting salt is well-known to those of ordinary skill in the art. Formulation of the desired fatty acid salt may then be carried out using techniques known to the ordinary skilled artisan.

The present invention includes within its scope pharmaceutical compositions comprising, as an active ingredient, any of the peptide agonist or antagonist fatty acid salt described herein in association with a pharmaceutically acceptable carrier delivery system suitable for transdermal administration. The present invention also includes its scope pharmaceutical compositions comprising, as an active ingredient, at least one peptide agonist or antagonist fatty acid salt disclosed herein as well as at least a second peptide agonist or antagonist fatty acid salt in association with a pharmaceutically acceptable carrier delivery system suitable for transdermal administration. For example, the present invention includes its scope pharmaceutical compositions comprising, as an active ingredient, at least one SSTR-2 agonist or antagonist fatty acid salt as well as at least a second SSTR agonist or antagonist fatty acid salt wherein said second SSTR agonist may be a fatty acid salt of an SSTR-1, SSTR-2, SSTR-3, SSTR-4 and/or an SSTR-5 agonist or antagonist in association with a pharmaceutically acceptable carrier delivery system suitable for transdermal administration.

In general, an effective dosage of active ingredient in the compositions of this invention may be varied; however, it is necessary that the amount of the active ingredient be such that a suitable dosage form is obtained. The selected dosage depends upon the desired therapeutic effect, the flux of the drug out of the TDP, the flux of the drug into and through the SC and other skin layers, and on the duration of the treatment, all of which are within the realm of knowledge of one of ordinary skill in the art. Generally, dosage levels of between 0.0001 to 100 mg/kg of body weight daily are administered to humans and other animals, e.g., mammals.

A preferred dosage range is 0.01 to 10.0 mg/kg of body weight daily, which can be administered as a single dose or divided into multiple doses.

### EXAMPLES

### 1. Synthesis of Somatostatin Agonists

The methods for synthesizing peptide somatostatin agonists are well documented and are within the ability of a person of ordinary skill in the art. For example, Compound A (D-2-Nal-c(Cys-Tyr-D-Trp-Lys-Val-Cys)-Thr-NH₂), also known as lanreotide, is readily prepared according to the procedure disclosed in U.S. Pat. No. 4,853,371, or the procedure disclosed in U.S. Pat. No. 5,411,943, the teachings of which are incorporated herein by reference. Lanreotide is currently marketed as the acetate salt in a 30 mg long-acting form and is available as Somatuline® Autogel from Ipsen Pharmaceuticals, Paris, France.

Synthesis of other somatostatin agonists, such as H-D-Phe-Phe-Phe-D-Trp-Lys-Thr-Phe-Thr-NH₂, can be achieved by following the protocol set forth in Example I of European Patent Application 0 395 417 A1. The synthesis of somatostatin agonists with a substituted N-terminus can be achieved, for example, by following the protocol set forth in PCT Publication No. WO 88/02756, PCT Publication No., WO 94/04752, and/or European Patent Application No. 0 329 295.

Peptides can be and were cyclized by using iodine solution in MeOH/water and purified on C18 reverse-phase preparative HPLC using acetonitrile-0.1% TFA/water-0.1% TFA buffers. Homogeneity was assessed by analytical HPLC and mass spectrometry and determined to be >95% for each peptide.

### 2. Synthesis of Somatostatin Agonists Di-Fatty Acid Salts

### 2.1 Compound A (D-2-Nal-c(Cys-Tyr-D-Trp-Lys-Val-Cys)-Thr-NH₂) fatty acid salts

Approximately 1.65 g of free base D-2-Nal-c(Cys-Tyr-D-Trp-Lys-Val-Cys)-Thr-NH₂ acetate salt (1.51 nmol (M.W.=1096 g/mol)) was dissolved in 160 ml deionized water and 16 ml ethanol. A 2 times molar excess solution of fatty acid counter ion in sodium salt form was prepared and cooled on ice. In this example, 0.92 g (or 3.02 mmoles) of oleic acid sodium salt (M.W.=304.45 g/mol) was dissolved in 80 ml deionized water and 8 ml ethanol.

The peptide and the fatty acid sodium salt solutions were mixed with stirring. To maximize formation and isolation of the fatty acid salt, the pH was adjusted to between 7 and 8. After a 10 minute incubation on ice, the dispersion was recovered on filter paper by vacuum filtration. The filter cake was rinsed with 100 ml of 10/1 (v/v) deionized water/ethanol in order to remove any unreacted peptide and fatty acid counter ion. The cake was frozen and vacuum dried for two days, whereupon a free-flowing white powder was obtained.

The following fatty acids in sodium salt form were used in the synthesis of D-2-Nal-c(Cys-Tyr-D-Trp-Lys-Val-Cys)-Thr-NH₂ salts:

Octanoic acid (Caprylic acid); a C8 saturated fatty acid with M.W.=144.21 g/mol and Tm=16° C.

Nonanoic acid (Pelargonic acid); a C9 saturated fatty acid with M.W.=158.24 g/mol and Tm=12° C.

Decanoic acid (Capric acid); a C10 saturated fatty acid with M.W.=172.27 g/mol and Tm=32° C.

Dodecanoic acid (Lauric acid); a C12 saturated fatty acid with M.W.=200.32 g/mol and Tm=43° C. Tetradecanoic acid (Myristic acid); a C₁₋₄ saturated fatty acid with M.W.=228.38 g/mol and Tm=54° C.

cis-9-Octadecanoic acid (Oleic acid); a C18 monounsaturated fatty acid with M.W.=282.47 g/mol and Tm=13° C.

cis,cis-9,12-Octadecadienoic acid (Linoleic acid); a C18 diunsaturated fatty acid with M.W.=280.45 g/mol and Tm=−9° C.

### 2.2 Compound B ([4-(2-hydroxyethyl)]-1-piperazinylacetyl-D-Phe-c(Cys-Tyr-D-Trp-Lys-Abu-Cys)-Thr-NH₂) fatty acid salts

Approximately 2 grams of free base [4-(2-hydroxyethyl)]-1-piperazinylacetyl-D-Phe-c(Cys-Tyr-D-Trp-Lys-Abu-Cys)-Thr-NH₂ acetate salt (1.66 nmol (M.W.=1202 g/mol)) was dissolved in 10 ml deionized water and 16 ml ethanol. A 2 times molar excess solution of fatty acid counter ion in sodium salt form was prepared. In this example, 1.01 g (or 3.32 mmoles) of oleic acid sodium salt (M.W.=304.45 g/mol) was dissolved in 60 ml deionized water.

The peptide and the fatty acid sodium salt solutions were mixed with stirring. To maximize formation and isolation of the fatty acid salt, 0.1M NaOH was used to adjust the pH to between 7 and 8. After a 10 minute incubation, the dispersion was recovered on filter paper by vacuum filtration. The filter cake was rinsed with 100 ml of deionized water in order to remove any unreacted peptide and fatty acid counter ion. The cake was frozen and vacuum dried for two days whereupon a free-flowing white powder was obtained.

TABLE 2

| Somatostatin-Fatty Acid Salt | % of Threoretical Yield Obtained |
|---|---|
| Compound A di-octanoate | 77.56 |
| Compound A di-nonanoate | 89.08 |
| Compound A di-decanoate | 79.94 |
| Compound A di-laurate | 71.05 |
| Compound A di-myristate | 68.86 |
| Compound A di-oleate | 83.69 + 3.90* |
| Compound A di-linoleate | 53.19 |
| Compound B di-oleate | 64.32 + 4.63* |

*n = 4

### 3. Characterization of Somatostatin Fatty-Acid Salts

Standard HPLC methods were used to qualitatively and quantitatively analyze Compound A, Compound B and the fatty acid moiety of the oleate salts. In one analysis, a POROS® Perfusion Chromatography™ column (Perseptive Biosystems; Framingham, Mass., U.S.A.) was used with a gradient of two mobile phases as follows:

TABLE 3

| | | % Eluant A | % Eluant B |
|---|---|---|---|
| | | (0.1% (v/v) TFA in | (0.1% (v/v) TFA in |
| Time (min) | Flowrate (ml/min) | Acetonitrile) | DI H2O) |
| 0.0 | 8 | 15 | 85 |
| 3.5 | 8 | 60 | 40 |
| 3.7 | 8 | 15 | 85 |

A theoretical potency was calculated on the basis of the formation of the di-fatty acid salt. For example, for Compound A, the anhydrous di-oleate salt would have a theoretical potency of 65.99% (w/w) (i.e. 1096/[1096+(2×282.47)]). Table 4 shows the actual potency obtained for each salt using this HPLC method compared to the theoretical potency for the di-salt in each case.

TABLE 4

| Somatostatin- | HPLC | Theoretical |
|---|---|---|
| Fatty Acid Salt | Potency (w/w) | Potency (w/w) |
| Compound A di-octanoate | 69.29% | 79.17% |
| Compound A di-nonanoate | 68.70% | 77.59% |
| Compound A di-decanoate | 63.53% | 76.08% |
| Compound A di-laurate | 60.29% | 73.23% |
| Compound A di-myristate | 65.98% | 70.58% |
| Compound A di-oleate | 59.47% + 5.89* | 65.99% |
| Compound A di-linoleate | 53.19% | 66.15% |
| Compound B di-oleate | 55.96% + 1.30* | 68.03% |

*n = 4

In another analysis, approximately 2 mg of somatostatin-fatty acid salt was weighed and dispersed in 2 ml acetonitrile; dissolution was achieved by addition of 2 ml of 0.1M trifluoroacetic acid. This mixture also allowed for dissociation of the peptide free base from the fatty acid counter ion. A Zorbax SB-C18 column (Hichrom Ltd., Reading, U.K.) was used.

TABLE 5

| | | % Eluant A | % Eluant B |
|---|---|---|---|
| | Flowrate | (79/21/0.1 (v/v/v) | (39/61/0.1 (v/v/v) |
| Time (min) | (ml/min) | H2O/Acetonitrile/TFA) | H2O/Acetonitrile/TFA) |
| 0.0 | 1 | 100 | 0 |
| 20.0 | 1 | 100 | 0 |
| 35.0 | 1 | 0 | 100 |
| 40.0 | 1 | 100 | 0 |

The quantity and purity of the oleic acid component of the fatty acid peptide salts was also determined. Approximately 5 mg of each sample was weighed and dissolved in 2 ml of acetonitrile plus 2 ml of 0.1M trifluoroacetic acid. An Inertsil ODS2 column (Hichrom Ltd., Reading, U.K.) and UV detection at 204 nm was employed. An isocratic elution of 90/10 (v/v) acetonitrile/DI H₂O at a flowrate of 1.5 ml/min was used; oleic acid purity was calculated as the percentage of total peak area made up by the oleic acid peak. Table 6 shows the average values (n=4) obtained for peptide and oleate content, and purity by these methods.

TABLE 6

| | | | Peptide | | | Oleate |
|---|---|---|---|---|---|---|
| Di-oleate | Peptide | Peptide | Content | Oleate | Oleate | Content |
| Salt | Purity | Content | (T*) | Purity | Content | (T*) |
| Compound A | 99.33% | 56.04% | 65.99% | 98.86% | 43.79% | 34.01% |
| | (+0.11) | (+0.74) | | (+0.15) | (+1.86) | |
| Compound B | 99.40% | 53.71% | 68.03% | 98.88% | 49.22% | 31.97% |
| | (+0.21) | (+5.21) | | (+0.10) | (+3.11) | |

(T*) = theoretical

¹H nuclear magnetic resonance profiles for both the acetate and oleate salts of Compound A and Compound B were obtained by dissolving approximately 10 mg of each salt in 0.5 ml deuterated dimethyl sulfoxide; analysis was carried out using a 400 MHz NMR (Bruker, Zurich, Switzerland).

¹³C nuclear magnetic resonance profiles for both the acetate and oleate salts of Compound A and Compound B were obtained by dissolving approximately 10 mg of each salt in 0.5 ml deuterated dimethyl sulfoxide; analysis was carried out using a 400 MHz NMR (Bruker, Zurich, Switzerland).

Powder samples of acetate and oleate salts of Compound A and Compound B were mixed with potassium bromide and analyzed by diffuse reflectance infrared spectroscopy (Thermo-Nicolet Ltd., Warwick, U.K.).

### 4. Solubility of Somatostatin Fatty-Acid Salts

The solubilities of Compound A and Compound B oleate were determined in deionized water, phosphate-buffered saline (PBS; pH 7.4) and n-octanol. Analysis was carried out by placing an excess of each salt with 1 ml of the selected solvent into four 4 ml sealed HPLC vials. The samples were allowed to equilibrate at 37° C. for 24 hours with shaking. Each of the four dispersions was then filtered through a 0.2 μm nylon membrane (Pall Gelman, Ann Arbor, Mich., U.S.A.). Prior to HPLC analysis, the n-octanol samples were diluted 1:100 with methanol and the PBS and deionized water samples were diluted 1:10 in de-ionized water. Table 7 lists the solubilities of Compound A and Compound B oleate salts in deionized water, PBS and n-octanol.

TABLE 7

| | Solubility (mg/ml) | | |
|---|---|---|---|
| Salt | DI H2O | PBS | n-Octanol |
| Compound A di-oleate | 0.035 | 0.040 | 122.80 |
| Compound B di-oleate | 0.050 | 1.302 | 136.90 |

### 5. Partition Properties of Somatostatin Fatty-Acid Salts

The partition coefficients for all somatostatin-fatty acid salts in either n-octanol/deionized water or n-octanol/PBS (pH 7.4) were determined. The partition coefficient is the concentration of Compound A or Compound B in the n-octanol phase divided by the concentration in the aqueous or PBS phase.

An accurately weighed sample of each salt was placed into 4 ml HPLC vials sealed with caps containing pierceable silicone septa and dissolved in 2 ml n-octanol. Two ml of either deionized water or PBS were added to each sample; the vials were vortexed vigorously and incubated at 37° C. with agitation for 24 hours in a shaker-incubator at 400 rpm.

Following incubation, two samples were withdrawn from each vial. Once the layers had fully partitioned, a syringe was used to draw a sample of the aqueous phase or the n-octanol phase. Samples from each phase were diluted appropriately with deionized or methanol prior to HPLC analysis. Tables 8 and 9 show the octanol/water or octanol/PBS partition coefficients, respectively.

TABLE 8

| | O/W | |
|---|---|---|
| Somatostatin Salt | Coefficient (KOW) | Log KOW |
| Compound A di-acetate | 0.50 | −0.301 |
| Compound A di-octanoate | 622.86 | 2.794 |
| Compound A di-nonanoate | 528.50 | 2.723 |
| Compound A di-decanoate | 211.53 | 2.325 |
| Compound A di-laurate | 876.40 | 2.943 |
| Compound A di-myristate | 407.50 | 2.610 |
| Compound A di-oleate | 598.14 | 2.777 |
| Compound A di-linoleate | 82.52 | 1.917 |
| Compound B di-acetate | 0.01 | −2.000 |
| Compound B di-oleate | 340.43 | 2.532 |

TABLE 9

| | O/PBS Coefficient | |
|---|---|---|
| Somatostatin Salt | (KOPBS) | Log KOPBS |
| Compound A di-acetate | 2.25 | 0.352 |
| Compound A di-octanoate | 6.71 | 0.827 |
| Compound A di-nonanoate | 6.47 | 0.811 |
| Compound A di-decanoate | 6.99 | 0.844 |
| Compound A di-laurate | 7.15 | 0.854 |
| Compound A di-myristate | 6.40 | 0.806 |
| Compound A di-oleate | 5.29 | 0.723 |
| Compound A di-linoleate | 7.35 | 0.866 |
| Compound B di-acetate | 0.01 | −2.000 |
| Compound B di-oleate | 0.04 | −1.398 |

### 6. In Vitro Permeation Studies

### 6.1 Preparation of Skin Specimens

The abdominal skin from male hairless mice (mice of the HRS strain carrying a mutant Hr gene (hr/hr)) aged 6-8 weeks was cleaned of any subcutaneous fat with blood vessels while maintaining the integrity of the viable epidermis and SC. The excised skin was used immediately after preparation and was not stored prior to use.

The viable epidermal layer was removed from the flanks of freshly sacrificed four month-old female domestic pigs. The flank area of each animal was carefully shaved and the upper 200 μm of skin (Meyer, W., et al., Curr. Prob. Dermatol., 7: 39-52, 1978; de Jalón, et al., J. Control. Rel., 75:191-197, 2001) was harvested using an electric dermatome (Robbins Instruments, Chatham, N.J., U.S.A.). Pigskin epidermal specimens were frozen at −18° C. between layers of PBS-soaked tissue paper enclosed in aluminum foil for up to two weeks prior to use in in vitro permeation studies.

### 6.2 Permeation Studies

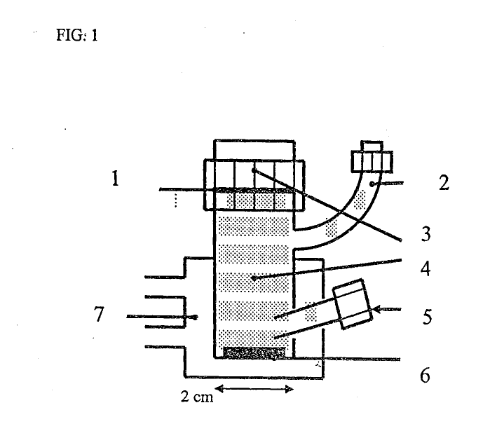

A custom made finite dosing diffusion cell (AGB Scientific, Dublin, Ireland, FIG. 1) based upon and modified from the Keshary-Chien type diffusion cell was used for in vitro permeation studies. This type of cell is thought to be superior to the Franz type cell with respect to maintaining the target skin surface body temperature in the receptor solution and also in its mixing efficiency (Keshary, P. R., et al., Drug Develop. Ind. Pharm., 10:883-913, 1984).

Three to five diffusion cells were used in each permeation experiment and monitoring was performed over a 48 hour period. The cells were linked in series to a thermostatically controlled circulation bath set at 35° C. to mimic human skin surface temperature. Each cell was placed on a stir-plate set at a defined stirring rate of 1,000 rpm. The surface area for permeation provided by each cell was 3.14 cm².

Data collected from each permeation study included:

1) the average maximum flux obtained in 48 hours (Jmax),

2) the total absorption characterized as the total weight of peptide delivered transdermally into the receiver compartment over a 48 hour period,

3) the lag time to detectable flux which was equivalent to the latest sampling time at which no peptide was detectable by HPLC analysis of the receiver solution, and

4) an equivalent 48-hour steady state flux was determined by obtaining the slope of the best-fit line for a plot of cumulative flux per unit area versus time (described by McDaid, D. M. et al., Int. J. Pharm., 133:71-83, 1996).

Fresh hairless mouse skin samples or fresh or freshly thawed pig skin samples were mounted onto the diffusion cell with the SC topmost and in contact with the donor system. A rubber or plastic ring was placed on top of the skin sample to secure it in position. The combination of the diffusion cell top, the skin and the rubber ring was securely fastened together by means of a threaded cap. A recorded volume of receiver solution was added to the receiver compartment and maintained at 35° C. The receiver solution was 60/40 (v/v) PBS/propylene glycol (pH 7.1). The propylene glycol fraction was used to increase the solubility of the somatostatin-fatty acid salts. Previous work reported by Morgan et al. (J. Pharm. Sci., 87:1213-1218, 1998) has shown that a 50/50 water/propylene glycol receptor solution had no detrimental effect on the barrier integrity of both hairless mouse and porcine skin.

The receiver solution was allowed to equilibrate at 35° C. for 30 minutes. The experiment was initiated by the application of 1 ml of donor solution containing approximately 5 mg free base peptide in DMSO to the skin surface followed by sealing of the donor compartment with Parafilm®. A narrow-bore hypodermic needle was used to withdraw 1 ml samples from the receiver compartment via the sampling port; at each sampling time, an equal volume of drug-free receiver solution was immediately added back to the receiver compartment. Samples were analyzed by HPLC.

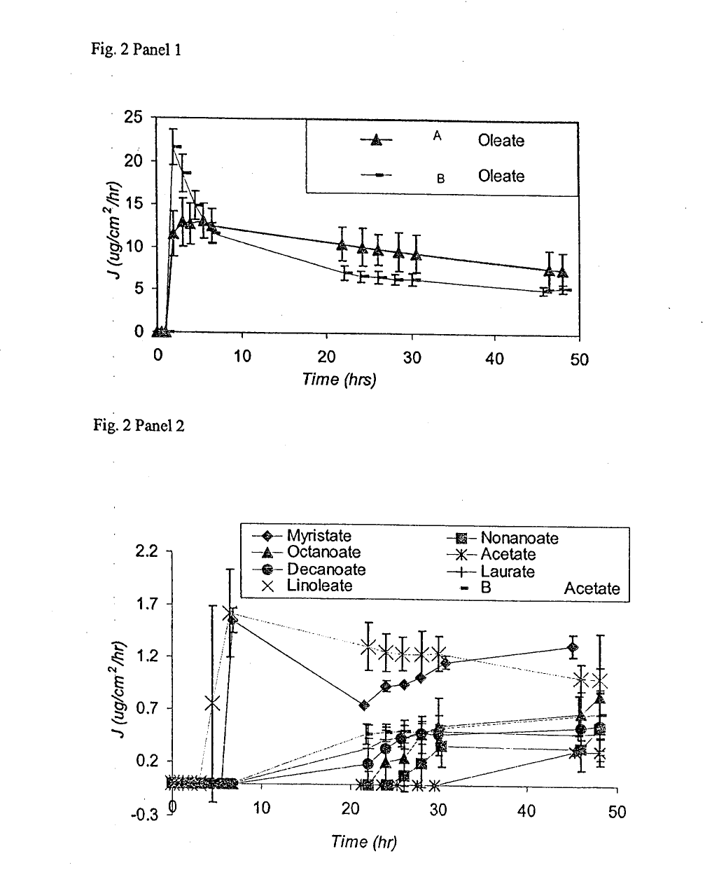

HPLC determination of peptide content in the receiver solution along with the known surface area of skin used (3.14 cm²) allowed for calculation of transdermal flux (in units of μg/cm²/hr) for each time point. Panels 1 and 2 of FIG. 2 show flux profiles obtained for the oleate salts of Compound A and Compound B in DMSO which far exceeded the flux profiles for the myristate, octanoate, decanoate, linoleate, nonanote, acetate and laurate salts of Compound A as well as the acetate salt of Compound B in DMSO.

### 7. Formulation Effects

### 7.1 Drug Concentration

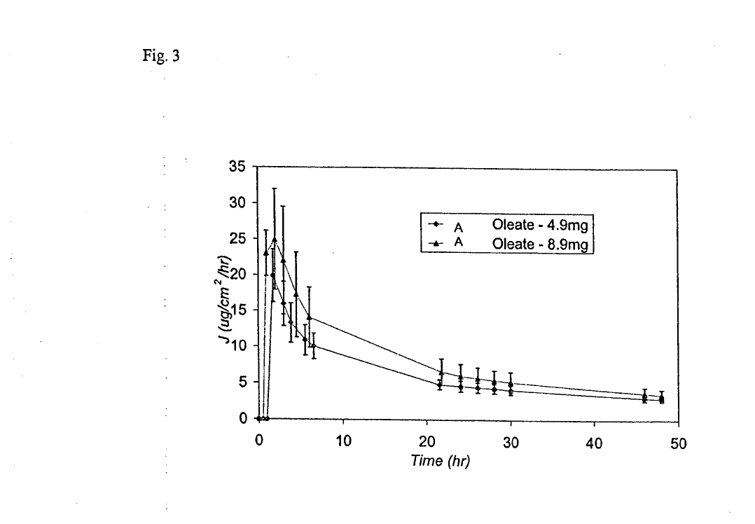

The effect of donor drug concentration was investigated across hairless mouse skin using Compound A oleate at 4.9 and 8.9 mg free base peptide per ml in DMSO. FIG. 3 shows an overlay of the flux profiles obtained. Table 10 shows the comparison of parameters derived from in vitro flux data across hairless mouse skin for DMSO solutions of Compound A oleate. R² values indicate the goodness of fit of the Jss linear regression analysis.

TABLE 10

| Conc. of free base | | | | Total |
|---|---|---|---|---|
| Compound A in | Lag Time | Jmax | Jss | Absorption |
| DMSO | (hr) | (μg/cm²/hr) | (μg/cm²/hr) | (μg/48 hr) |
| 4.9 mg/ml | 1.0 | 19.887 | 2.858 | 448.52 |
| | | | (r² = 0.861) | |
| 8.9 mg/ml | 0.5 | 24.942 | 3.342 | 522.82 |
| | | | (r² = 0.818) | |

The quantity of peptide present in the upper SC and lower dermis layers of the hairless mouse skin samples were determined for each concentration of analog tested. To determine analog concentrations in the SC, the skin samples were wiped clean of donor solution, patted dry and stripped twenty times with Sellotape®, using a fresh piece for each stripping. The twenty pieces of Sellotape® were pooled in a 50 ml centrifuge tube and 10 ml of 50/50 (v/v) acetonitrile/0.1M TFA was added. The sample was homogenized at 20,000 rpm for 5 minutes and then filtered. The peptide content was determined using HPLC. To determine peptide content in the dermal layer, the remaining skin sample was cut into fine pieces, homogenized and analyzed as described.

Table 11 below shows the percentage of total applied dose obtained in the upper and lower skin layers for both experiments; a large amount of peptide is detected in the upper SC skin layers for the higher dose. Based on these results, a donor concentration of ˜5 mg/ml was used in subsequent experiments.

TABLE 11

| Weight of free base | | | |
|---|---|---|---|
| Compound A in | Wt. % in upper | Wt. % in lower | Wt. % |
| DMSO | skin layers | skin layers | delivered |
| 4.9 mg | 20.22% | 7.63% | 9.23% |
| 8.9 mg | 49.93% | 1.09% | 5.84% |

### 7.2 Solvents and Penetration Enhancers

Once lead candidate somatostatin-fatty acid salts had been selected on the basis of in vitro permeation in a DMSO solution, the effect of different solvents and penetration enhancers on permeation was assessed. The concentration of the donor solution corresponded to 5 mg/ml free base peptide for all experiments and all donor solutions were equilibrated at 37° C. for one hour prior to use. These experiments were carried out using hairless mouse skin and pigskin barriers.

The solvents assayed were propylene glycol and ethanol both alone and in combination with deionized water. Chemical penetration enhancers investigated were the fatty acid oleic acid, the terpene 1,8-cineole, the surfactant sodium lauryl sulphate, and the fatty alcohols decanol and dodecanol.

### 7.2.1 Donor Solvent

The effect of using DMSO, propylene glycol and aqueous ethanol as donor solvent was assessed for both peptides using the in vitro hairless mouse model as described above.

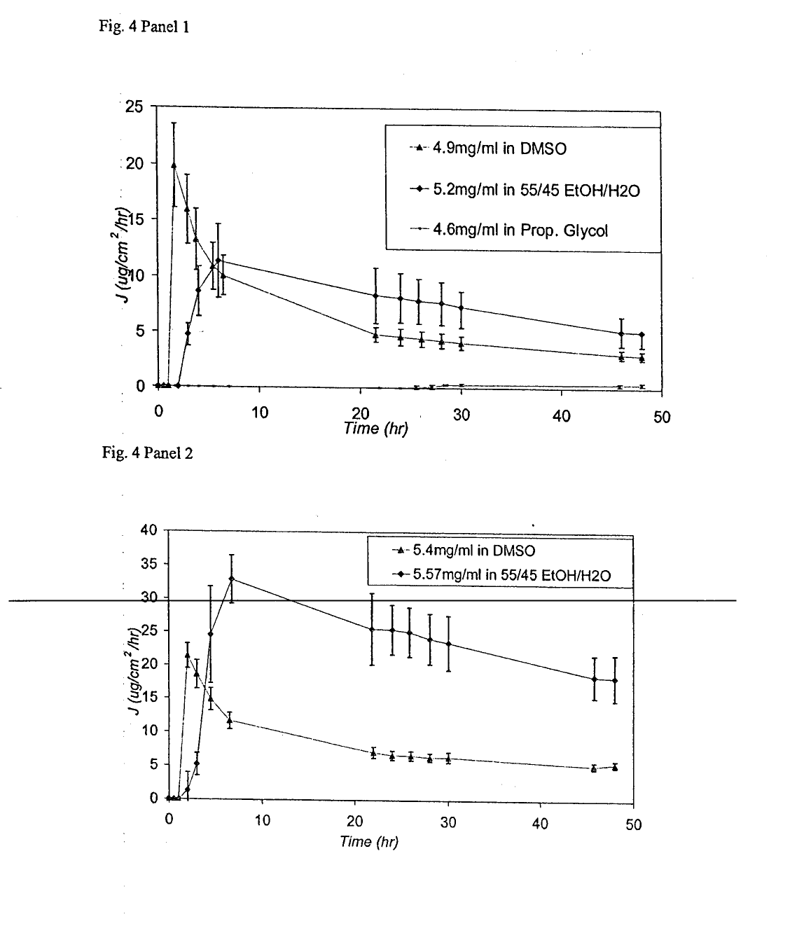

Panel 1 of FIG. 4 shows the flux profiles obtained for Compound A oleate at a free base peptide concentration of approximately 5 mg/ml in DMSO, propylene glycol or 55/45 (v/v) ethanol/water. Panel 2 of FIG. 4 shows the flux profiles obtained for Compound B oleate at a free base peptide concentration of approximately 5 mg/ml in DMSO or 55/45 (v/v) ethanol/water. The parameters derived from these profiles are included in Table 12. R² values indicate the goodness of fit of the Jss linear regression analysis.

TABLE 12

| | Compound A oleate | | | Compound B oleate | |
|---|---|---|---|---|---|
| | | 55/45 | Propylene | | 55/45 |
| Donor Solvent | DMSO | EtOH/H₂O | glycol | DMSO | EtOH/H₂O |
| Lag Time (hr) | 1 | 2 | 24 | 1 | 1 |
| Jmax | 19.887 | 11.343 | 0.366 | 21.407 | 32.902 |
| (μg/cm²/hr) | | | | | |
| Jss | 2.858 | 5.871 | 0.318 | 5.158 | 20.264 |
| (μg/cm²/hr) | (r² = 0.861) | (r² = 0.905) | (r² = 0.760) | (r² = 0.954) | (r² = 0.948) |
| Total | 448.52 | 770.02 | 55.17 | 814.86 | 2,750.01 |
| (μg/48 hr) | | | | | |
| Absorption | | | | | |

### 7.2.2 Penetration Enhancers

The effects of penetration enhancers oleic acid, sodium lauryl sulfate, 1,8-cineole decanol (C₁₀) or dodecanol (C₁₂) upon transdermal permeation were assessed for both peptides using the in vitro hairless mouse model as described above.

### Oleic Acid

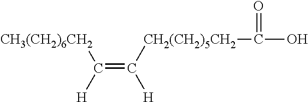

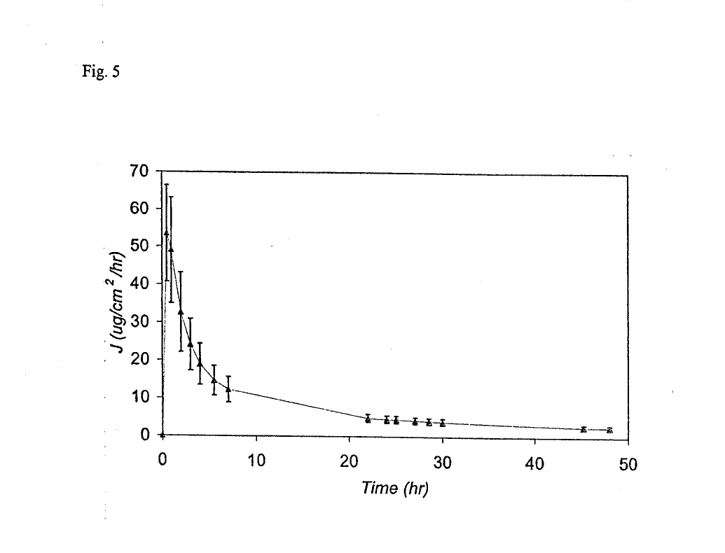

FIG. 5 shows the effect of 1% (v/v) free oleic acid as a transdermal enhancer for a donor solution consisting of Compound A acetate in DMSO. Table 13 shows the derived parameters for Compound A acetate in DMSO without oleic acid and Compound A oleate in DMSO. R² values indicate the goodness of fit of the Jss linear regression analysis. The use of oleic acid clearly leads to an increase of flux for D-2-Nal-c(Cys-Tyr-D-Trp-Lys-Val-Cys)-Thr-NH₂ acetate.

TABLE 13

| | Lag | | | Total |
|---|---|---|---|---|
| | Time | Jmax | Jss | Absorption |
| Donor Solution | (hr) | (μg/cm²/hr) | (μg/cm²/hr) | (μg/48 hr) |
| 5.2 mg/ml Compound | 29.5 | 0.320 | 0.240 | 48.08 |
| A acetate in DMSO | | | (r² = 0.561) | |
| 4.3 mg/ml Compound | 0.0 | 53.576 | 1.873 | 387.78 |
| A acetate in DMSO | | | (r² = 0.698) | |
| +1% (v/v) oleic acid | | | | |
| 4.9 mg/ml Compound | 1.0 | 19.887 | 2.858 | 448.52 |
| A oleate in DMSO | | | (r² = 0.861) | |

### Sodium Lauryl Sulphate

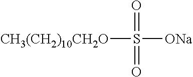

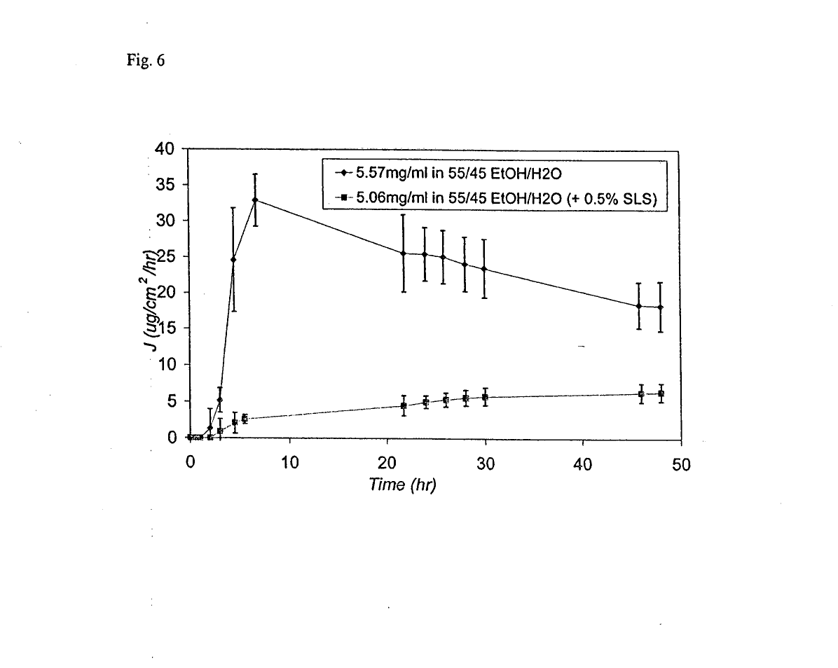

Another well characterized CPE, sodium lauryl sulphate (SLS), was assessed for its effect on the flux of [4-(2-hydroxyethyl)]-1-piperazinylacetyl-D-Phe-c(Cys-Tyr-D-Trp-Lys-Abu-Cys)-Thr-NH₂ oleate across hairless mouse skin in vitro. The donor solvent used was 55/45 (v/v) ethanol/water. SLS was used at a concentration of 0.5% (v/v) which is thought to be close to the maximum which can be used without causing significant skin damage (Patil, S., et al., J. Pharm. Sci., 84:1240-1244, 1995). FIG. 6 shows a comparison of the flux profiles with and without the SLS enhancer.

### 1,8-Cineole

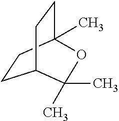

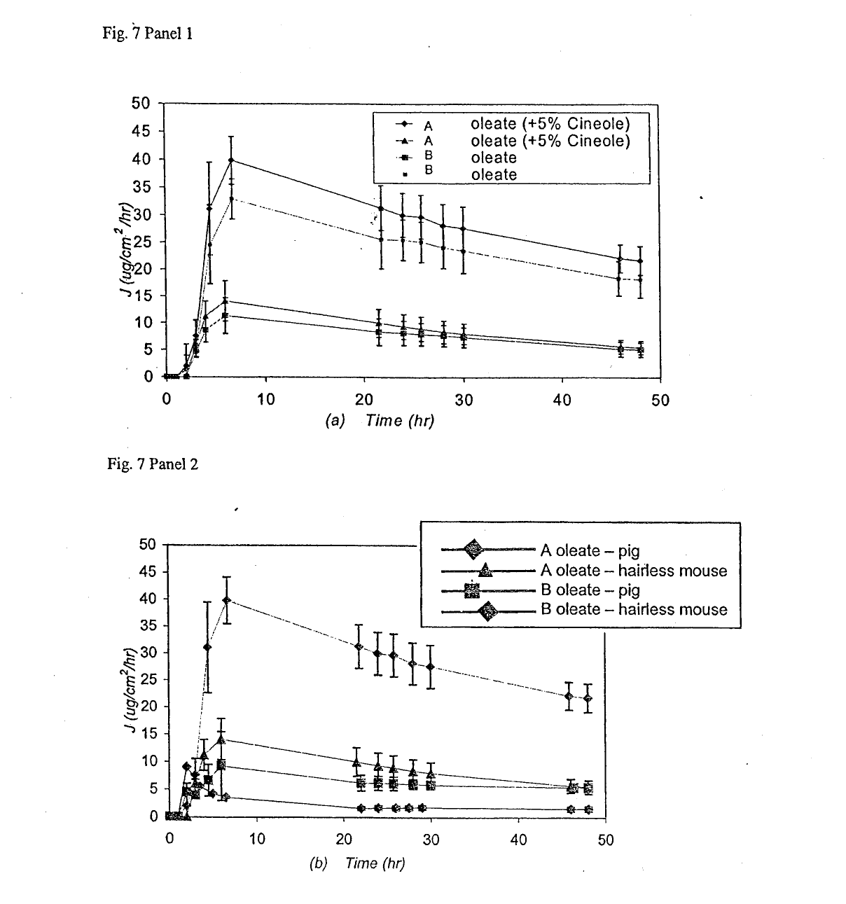

Both Compound A and Compound B oleate salts were assessed for in vitro transdermal flux across both hairless mouse skin and dermatomed pig epidermis using a donor solution consisting of 55/40/5 (v/v/v) ethanol/water/cineole. Panel 1 of FIG. 7 shows the flux profiles across hairless mouse skin for both salts with and without cineole in the donor solution, while Panel 2 of FIG. 7 compares the fluxes obtained for the cineole-containing donor solutions across both hairless mouse skin and pig epidermis.

The parameters derived from these profiles are presented in Tables 14 and 15. R² values indicate the goodness of fit of the Jss linear regression analysis. It can be seen that the use of cineole as a penetration enhancer does not alter the shape of the flux profile, nor does it lead to a decrease in the lag time. For both Compound A and Compound B oleate, however, there is a clear increase in flux due to this enhancer. For Compound A oleate, the total peptide flux across hairless mouse skin over 48 hours increased by 7.19% while for Compound B oleate the increase was 19.26%.

TABLE 14

| | Hairless Mouse Skin | | Pig Epidermis |
|---|---|---|---|
| | | 55% EtOH | 55% EtOH |
| | 55% EtOH | 40% Water | 40% Water |
| Donor Solvent | 45% Water | 5% Cineole | 5% Cineole |
| Lag Time (hr) | 2 | 2 | 1 |
| Jmax | 11.343 | 14.100 | 9.019 |
| (μg/cm²/hr) | | | |
| Jss | 5.871 | 6.359 | 1.407 |
| (μg/cm²/hr) | (r² = 0.905) | (r² = 0.880) | (r² = 0.945) |
| Total Absorption | 770.02 | 825.35 | 231.09 |
| (μg/48 hr) | | | |

TABLE 15

| | Hairless Mouse Skin | | Pig Epidermis |
|---|---|---|---|
| | | 55% EtOH | 55% EtOH |
| | 55% EtOH | 40% Water | 40% Water |
| Donor Solvent | 45% Water | 5% Cineole | 5% Cineole |
| Lag Time (hr) | 1 | 1 | 1 |
| Jmax | 32.902 | 39.838 | 9.224 |
| (μg/cm2/hr) | | | |
| Jss | 20.264 | 24.075 | 5.552 |
| (μg/cm2/hr) | (r2 = 0.948) | (r2 = 0.951) | (r2 = 0.990) |
| Total Absorption | 2,750.01 | 3,279.62 | 814.44 |
| (μg/48 hr) | | | |

### Fatty Alcohols

CH₃(CH₂)₈CH₂OH CH₃(CH₂)₁₀CH₂OH

Decanol (n-decyl alcohol) Dodecanol (Lauryl alcohol)

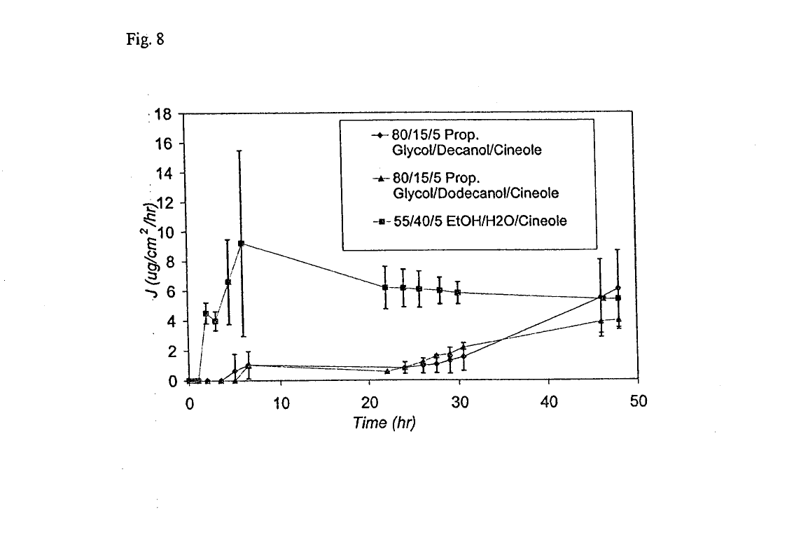

The effect of fatty acid alcohols on the in vitro transdermal flux of Compound B oleate across pig epidermis was determined; decanol and dodecanol were included at 15% in a donor vehicle containing 80% propylene glycol and 5% cineole. FIG. 8 shows the flux profiles obtained for both propylene glycol/fatty alcohol vehicles in comparison with that for 55/40/5 (v/v/v) ethanol/water/cineole. The derived parameters are presented in Table 16 below.

TABLE 16

| | | 80% Prop. | |
|---|---|---|---|
| | 55% EtOH | Glycol | 80% Prop. Glycol |
| | 40% Water | 15% Decanol | 15% Dodecanol |
| Donor vehicle | 5% Cineole | 5% Cineole | 5% Cineole |
| Lag Time (hr) | 1 | 3.5 | 5 |
| Jmax | 9.224 | 6.085 | 3.983 |
| (μg/cm²/hr) | | | |
| Jss | 5.552 | 4.539 | 3.335 |
| (μg/cm²/hr) | (r² = 0.990) | (r² = 0.671) | (r² = 0.780) |
| Total Absorption | 814.44 | 917.18 | 600.35 |
| (μg/48 hr) | | | |

As demonstrated herein, and as the skilled artisan would know and appreciate, a variety of solvents and enhancers are available for transdermal delivery and various combinations are tested during development of a transdermal delivery system. The skilled artisan would also know that different combinations of solvents and enhancers may result in similar transdermal flux profiles. Based upon the work from this study, while in no way limiting as to other combinations of vehicles, solvents, enhancers and formulation and delivery methods, a 55/40/5 (v/v/v) ethanol/water/cineole vehicle was chosen for further study. The steady state flux values for Compound A and Compound B oleate salts with this vehicle across pig epidermis are 1.407 and 5.552 μg/cm²/hr respectively. Using a patch area of 20 cm², it is estimated that this would equate to a daily systemic delivery of approximately 0.675 mg Compound A or approximately 2.665 mg Compound B across human skin.

### 7.3 Other Formulation Effects

The skilled artisan would also know and appreciate that other parameters may be considered in preparing a formulation for transdermal delivery. For example, vehicle pH, viscosity of the formulation, and patch design can, and will, effect transdermal penetration.

### 7.3.1 Donor Vehicle pH

The donor vehicles described herein were prepared at or close to neutral pH; this pH is close to the pH at which the fatty acid salts were synthesized and isolated. Because this work focused upon fatty acid peptide salts, a neutral pH was preferred to maintain molecular pairing. As described by Fini et al. (Int. J. Pharm., 187:163-173, 1999), adverse pH conditions can cause salt/ion pairs to dissociate, resulting in the loss of many properties beneficial to increasing transdermal flux, for example lipophilicity. Table 17 shows pH values measured for select vehicles; Compound B oleate was dissolved at approximately 5 mg/ml (based on free base peptide) in all vehicles prior to pH measurement. These values suggest that the peptides remained in the form of fatty acid salts following dissolution in the various donor vehicles. Significant dissociation would have led to distortion of the flux profiles due to the formation of free oleic acid, in itself a potent penetration enhancer.

TABLE 17

| Donor Vehicle | pH |
|---|---|
| 55% EtOH | 6.89 |
| 40% H2O | |
| 5% 1,8-Cineole | |
| 80% Prop. Glycol | 7.08 |
| 15% Decanol | |
| 5% 1,8-Cineole | |
| 80% Prop. Glycol | 7.56 |
| 15% Dodecanol | |
| 5% 1,8-Cineole | |

### 7.3.2 Donor Vehicle Viscosity

Vehicle viscosity may also be adjusted so as to minimize evaporation of volatile vehicle components such as ethanol and enhance transdermal permeation. Viscous vehicle formulations aid in patch assembly and reduce loss or leakage of donor solution. The selection and testing of viscosifying agents, such as hydroxypropyl cellulose, is well known to those skilled in the art. Others viscosifying agents include, but are not limited to, acrylates/C10-30 alkyl acrylate crosspolymers (e.g., Pemulen TR-1, Pemulen TR-2, Carbopol 1342, Carbopol 1382 and Carbopol ETD 2020) or cellulose derivative polymers (e.g., methylcellulose, ethylcellulose, hydroxyethylcellulose, hydroxyethyl ethylcellulose, hydroxypropyl methyl cellulose, nitrocellulose, sodium cellulose sulfate, sodium carboxymethylcellulose, crystalline cellulose, cellulose powder, and mixtures thereof).

### 8. Patch Design

The skilled artisan would know and appreciate that variation in patch design and materials can and will affect transdermal permeation. Patch design for in vivo experiments described herein included either a membrane-controlled or a matrix diffusion controlled device.

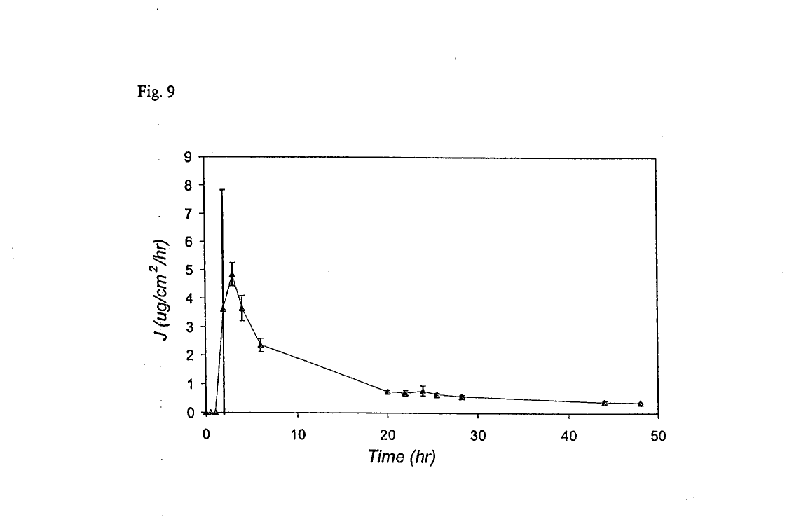

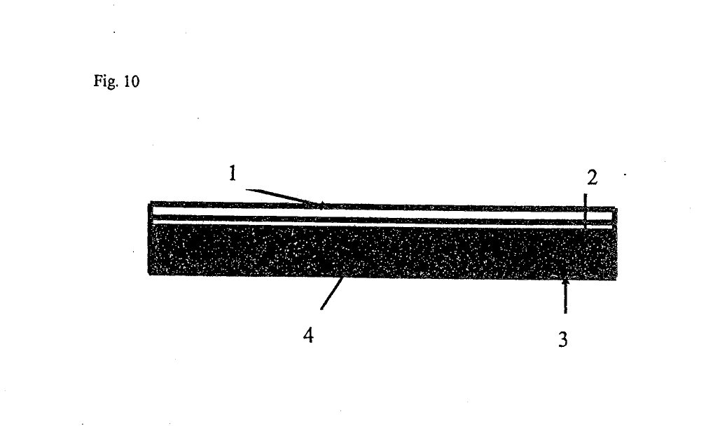

A membrane controlled device requires that the formulation pass across both a microporous membrane and an adhesive layer. Using in vitro methodology, a 55/40/5 (v/v/v) ethanol/water/cineole vehicle containing 5 mg/ml Compound B oleate was assayed across pig epidermis overlaid with a polypropylene microporous membrane. Vehicle viscosity was increased with 5% (w/v) 370,000 g/mol hydroxypropyl cellulose (HPC). FIG. 9 shows the flux profile obtained, while Table 18 compares the derived parameters with those obtained for the equivalent non-viscosified vehicle in the absence of a microporous polyproylene membrane.

TABLE 18

| | 55/45/5 | 55/45/5 EtOH/Water/Cineole + |
|---|---|---|
| Donor vehicle | EtOH/Water/Cineo | 5% (w/v) HPC |
| Microporous | No | Yes |
| Polypropylene | | |
| Lag Time (hr) | 1 | 1 |
| Jmax (μg/cm²/hr) | 9.224 | 4.200 |
| Jss (μg/cm²/hr) | 5.552 | 0.309 |
| | (r² = 0.990) | (r² = 0.512) |
| Total Absorption | 814.44 | 56.89 |
| (μg/48 hr) | | |

### 9. In Vivo Permeation Studies

Each in vivo study involved the use of female weanling domestic pigs aged 4 to 6 weeks and weighing approximately 10 Kg. The animals were housed separately and were maintained in accordance with the principles of good laboratory animal health.

### 9.1 Preparation of Transdermal Patches or Ointments

A matrix diffusion-controlled type patch was prepared and used for delivery of the somatostatin fatty acid salts. The backing membrane was heat sealable polyester film and a reservoir depth of 1 cm was provided by the use of foam tape (both from 3M Health Care, St. Paul, Minn., U.S.A.). A central circular depot with a diameter of 4 cm surrounded by a 1 cm wide (Type A) or 1.5 cm wide (Type B) border was formed. A ring of tackified, polyisobutylene base pressure sensitive adhesive (25 μm thick, Adhesives Research Inc., Limerick, Ireland) was adhered to the foam border ring.

Ointments of the oleate salts of Compound A and Compound B were also assayed in vivo. Four ointment bases were investigated: Macrogol Ointment BP, Wool Alcohols Ointment BP, Plastibase Ointment and Emulsifying Ointment BP (Department of Pharmaceutics and Pharmaceutical Technology, School of Pharmacy, Trinity College Dublin, Ireland).

The salts were dissolved at 20 mg free base per ml in 0.2 ml ethanol and 0.05 ml 1,8-cineole and the 0.25 ml somatostatin fatty acid salt solution was drawn into a 1 ml syringe. The ointment bases were heated to approximately 40° C. and 0.75 ml of each was then drawn up into the syringe already containing 0.25 ml somatostatin fatty acid salt solution; the syringes were immediately capped and mixed by vortexing. Each syringe, therefore, contained 1 ml of an ointment containing 5 mg of Compound A or Compound B (as oleate salt) in 75% ointment base, 20% ethanol, and 5% 1,8-cineole. All doses were stored in the syringe at 4° C. until use.

### 9.2 Application of Patches or Ointments

In all trials, the transdermal patches or ointments were applied to the shaven flank of a weanling pig.

When using matrix patch Type A, 1 ml of donor solution containing 5 mg/ml Compound A or Compound B as oleate salt in 95/5 (v/v) ethanol/cineole gelled with 2.5% (w/v) HPC was applied to cotton wool residing in the central depot of the patch, the adhesive release liner was removed, and the device was pressed firmly onto the shaven area. An adhesive bandage (Lohmann, Neuwied, Germany) was then placed over the entire patch to minimize dislodgment. When using matrix patch Type B, 1 ml of donor solution was applied directly onto the impermeable backing membrane in the central depot of the patch, the adhesive release liner was removed and the device was pressed firmly onto the shaven area. An adhesive bandage (Lohmann, Neuwied, Germany) was then placed over the entire patch to minimize dislodgment. The donor solution was in direct contact with the skin over a surface area of 12.56 cm².

For direct application of the somatostatin analog fatty acid salts in ointment form, a 4 cm diameter circle was marked on the shaven flank area of each pig and a 1 ml sample of each ointment was placed in the marked off area and worked into the entire area evenly using a gloved finger. The site was then occluded using a polyester backing membrane (3M Health Care, St. Paul, Minn., U.S.A.) held in place by an adhesive bandage (Lohmann, Neuwied, Germany).

### 9.3 Sampling and Analysis

A drug is generally considered as having been “transdermally delivered” once it passes the epidermis-dermis junction as the dermis layer is a vascularized tissue. Blood samples were taken from each weanling pig at approximately 6, 24, 48, and 72 hours after patch or ointment application. Approximately 1 ml of blood was drawn from the jugular vein at each sampling time into a sterile 2 ml syringe containing 0.1 ml of heparin to prevent blood clotting (Heparin (Mucous) Injection BP, Leo Laboratories Ltd., Dublin, Ireland). Following centrifugation at 3,000 rpm for 5 minutes, plasma was isolated from each blood sample and stored at −18° C. prior to analysis.

### 9.3.1 Radioimmunoassay

A calibration curve covering a concentration range of 9.8 to 62.5 pg/ml was prepared for the acetate salt peptides of Compound A and Compound B. The RIA buffer used for dilution of both the standards and plasma test samples was a 100 mM potassium phosphate buffer, pH 7.4, containing 0.2% (w/v) BSA, 0.9% (w/v) NaCl, 0.01% sodium azide and 0.1% Triton X-100. Plasma samples were diluted appropriately with RIA buffer so that the radioactivity reading fell within the optimal part of the calibration curve.

Duplicate 50 μl aliquots of each standard and each sample were placed in separate gamma counter tubes (Sarstedt, Nümbrecht, Germany). To each tube was added 100 μl of RIA buffer followed by 100 μl of iodinated peptide solution corresponding to approximately 10,000 cpm/tube and 100 μl of antibody serum diluted 1:700. Control tubes to measure non-specific (NS) binding, maximum binding (B0), and total activity (TA) were prepared in triplicate. The NS and TA samples lacked antibody serum and the BO samples lacked iodinated peptide. All tubes were incubated at 4° C. for 48 hours.

At the end of the incubation period, the free fractions (^{[125]}peptide) and bound fractions (^{[125]}peptide-antibody complex) were separated by precipitation with 1 ml of 1-propanol at a temperature range of 0-4° C. The precipitate was isolated by centrifuging at 3,000 rpm for 30 minutes and decanting off the supernatant. The radioactivity of the residues, corresponding to the bound fractions was then counted in a gamma counter (Packard Bioscience Company, Meriden, Conn., U.S.A.).

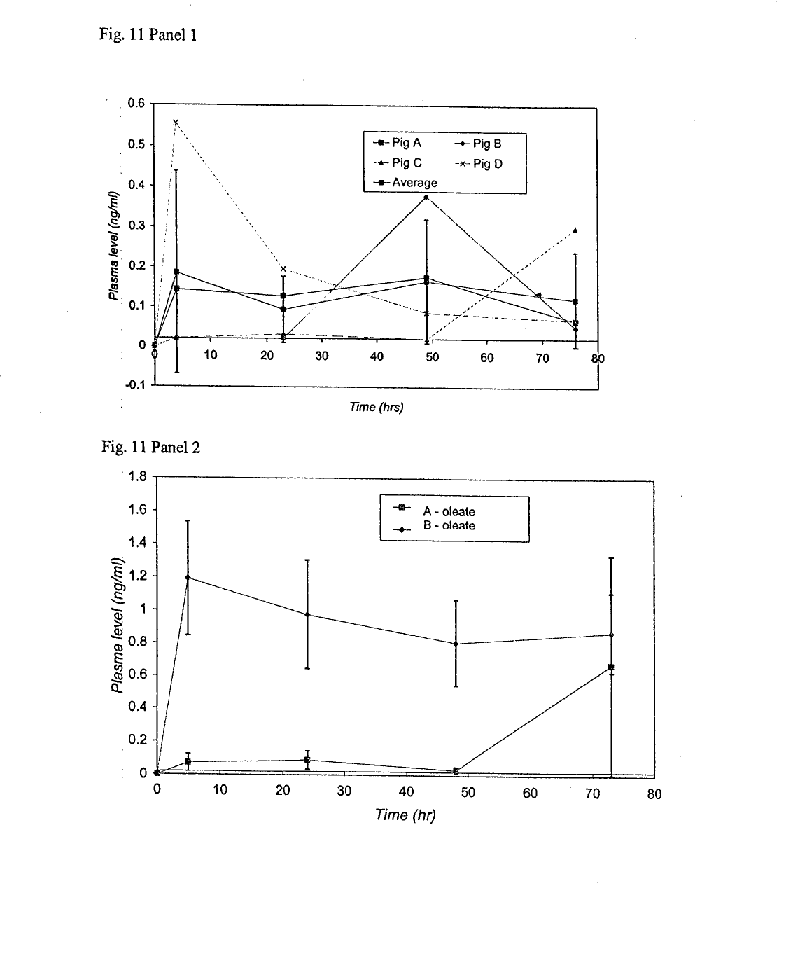

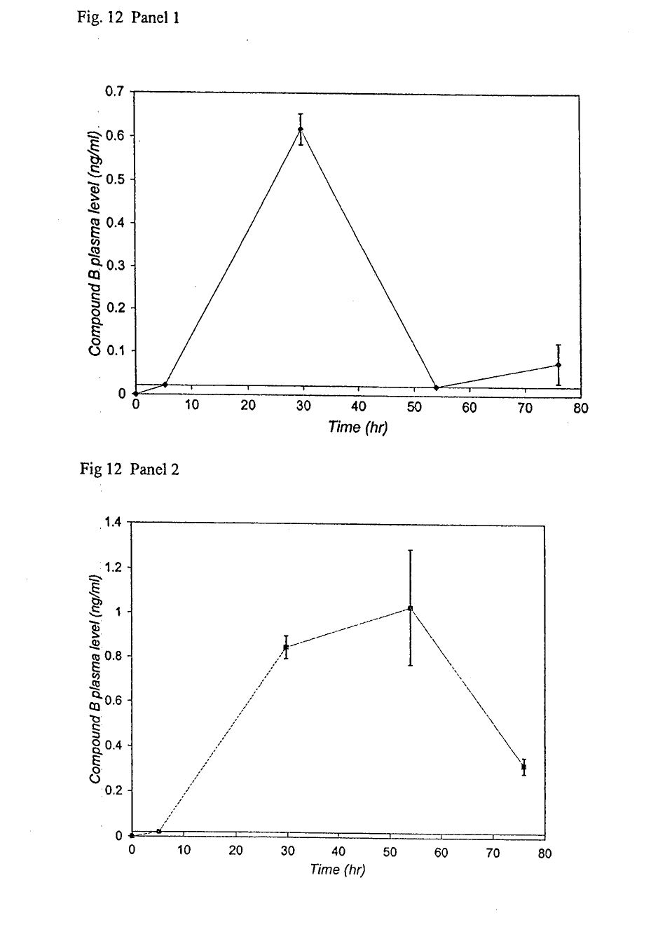

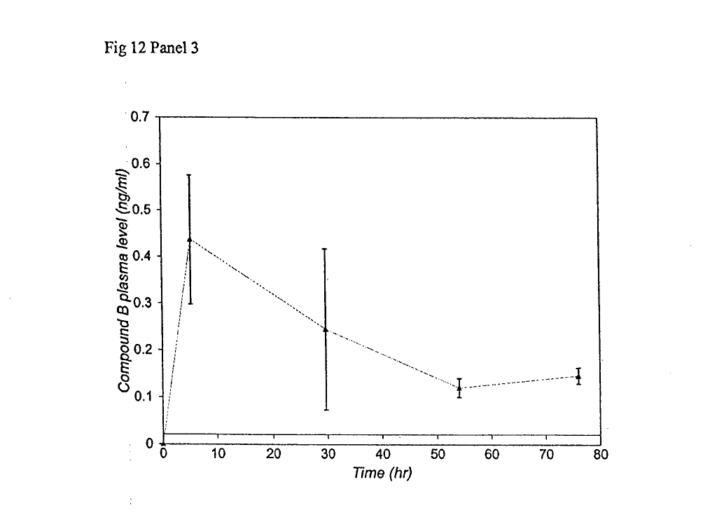

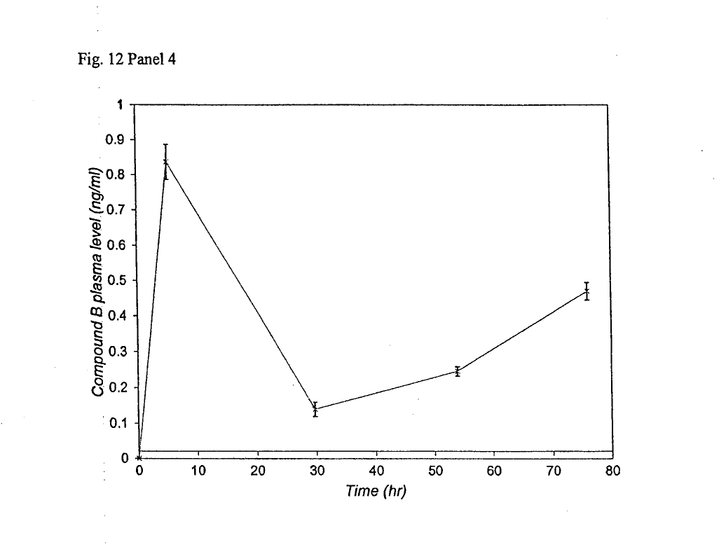

Since the principle of the RIA is the competition between iodinated and non-iodinated peptide for occupation of the antibody binding sites, the quantity of iodinated peptide bound, i.e. the radioactivity of the residue, is inversely proportional to the quantity of non-iodinated peptide present from the plasma or standard sample. The response for each time point was normalized in relation to the maximum B0 binding after subtraction of the NS binding. The percent bound was determined by averaging the count of each duplicate standard or sample, subtracting the average non-specific binding counts (NS), and dividing these corrected counts by the corrected B0 counts. Panels 1 and 2 of FIG. 11 show the Compound B plasma profiles obtained after patch delivery while FIGS. 12A, 12B, 12C and 12D show the Compound B plasma profiles obtained after ointment delivery. Table 19 shows the pharmacokinetic data derived from all in vivo experiments. AUC refers to area under the plasma concentration-time curves calculated by the trapezoidal rule.

TABLE 19

| | | | AUC0 [symbol] |
|---|---|---|---|
| IVV Experiment | Cₘₐₓ (ng/ml) | tₘₐₓ (h) | 72 (ng/ml)/hr |
| Compound B Patch A | 0.185 | 4.00 | 10.30 |
| Compound B Patch B | 0.671 | 73.00 | 11.70 |
| Compound A Patch B | 1.191 | 5.00 | 65.30 |
| Compound B | 0.618 | 29.75 | 30.40 |
| Wool Alcohols Ointment | | | |
| Compound B Plastibase | 1.029 | 54.00 | 48.60 |
| Compound B | 0.437 | 5.25 | 16.90 |
| Emulsifying Ointment | | | |
| Compound B Macrogol | 0.837 | 5.25 | 26.20 |
| Ointment | | | |

Inspection of patch Type A at the end of the experiment revealed that all patches were at least partially detached from the pigskin. This resulted in evaporation of the ethanol-based donor vehicle; as such, the formulation may only have been in contact with the skin for a relatively short period at the start of the experiment. It is clear that oleate fatty acid salts of somatostatin analogs, however, are able to transverse the skin and are delivered into the circulatory system of the host via a transdermal patch system.

### 10. Statistical Analysis

Standard statistical analysis tools were used to analyze the data. The skilled artisan would know and recognize which analysis tools are most suitable to analyze the permeability data particular to a set of study conditions. Raw data for cumulative amount of fatty acid peptide salt transferred versus time was analyzed by linear regression; a best fit line was obtained for each experiment with the goodness of fit of the line described by the parameter r² where:

r=Sxy/\sqrt{Sxx·Sxy}

r²=r×r Eqn. 10.1

whereby

Sxy=Σxy−(Σx·Σy/n) Eqn. 10.2

Sxx=Σx2−(Σx2/n) Eqn. 10.3

Syy=Σy2−(Σy2/n) Eqn. 10.4

When comparing multiple sets of data, the standard 2 sample t test at a significance level of 95% was employed. The data was judged to be significantly different when the p value was <0.05. The values for the mean and the standard deviation (using n−1) as quoted are those used in normal practice. When comparing two sets of data, it was assumed that the data followed a normal distribution about a true mean. In the event that the distribution of the data is not normal, non-linear regression analysis may be used.

A number of basic equations exist for the prediction of drug plasma levels in humans obtained by transdermal delivery. In order to make use of these equations, basic human pharmacokinetic parameters are required for the drug in question. Chassard et al. (J. Endocrinol. Invest. 20(Supp. to no. 7):30-32, 1997) reported on the pharmacokinetics of Compound A after i.v. infusion of 7, 21 and 42 μg/Kg to healthy human volunteers (n=12) obtaining values as shown in Table 20.

TABLE 20

| Parameter | Units | 7 μg/Kg | 21 μg/Kg | 42 μg/Kg |
|---|---|---|---|---|
| t_{1/2} | hr | 0.8 ± 0.1 | 1.0 ± 0.1 | 1.6 ± 0.3 |
| AUC | (ng/ml)/hr | 21.2 ± 4.1 | 74.3 ± 13.7 | 180.6 ± 46.6 |
| MRTinfusion | hr | 0.7 ± 0.1 | 0.8 ± 0.1 | 0.9 ± 0.2 |
| CL | (ml/hr)/Kg | 341 ± 62 | 292 ± 58 | 249 ± 72 |
| Vₛₛ | ml/Kg | 194 ± 41 | 186 ± 44 | 187 ± 49 |
| Kₑₗ | hr⁻¹ | 0.87 ± 0.11 | 0.69 ± 0.07 | 0.43 ± 0.09 |

The values determined for elimination half-life (t_{1/2}), area under the curve (AUC), mean residence time (MRT), plasma clearance (CL), elimination rate constant (Kₑₗ) and volume of distribution in steady state conditions (Vₛₛ) confirmed non-linear behaviour for D-2-Nal-c(Cys-Tyr-D-Trp-Lys-Val-Cys)-Thr-NH₂ in the range of administered doses (7-42 μg/Kg) studied. The values obtained for the intermediate dose of 21 μg/Kg were used to determine the necessary input rate, i.e., steady state flux, required in humans to achieve therapeutic plasma levels as outlined by Lee et al. (Res. Comm. Mol. Path. Pharmacol., 85, 337-346, 1994) in their studies on melatonin. A V_{d} value was determined from the Vss value, normalized for subject weight, by multiplying by 70 Kg, giving a value of 13,020 ml. As described by Chatterjee et al. (Pharm. Res., 14:1058-1065, 1997), the following equation can be used to predict required delivery rates, assuming transdermal delivery can be treated as an intravenous infusion:

K₀=Cₚₛₛ·Kₑₗ·V_{d} Eqn. 10.5

where K₀ is the required zero order delivery rate to achieve a therapeutic steady state plasma concentration (Cₚₛₛ). For Compound A with a required Cₚₛₛ=1 ng/ml, the required input rate is 0.001 μg/ml×0.69 hr⁻¹×13,020 ml, or 8.98 μg/hr. Thus, for a skin area of 20 cm², the required steady-state transdermal flux would be 0.45 μg/cm²/hr. Assuming a therapeutic plasma level of 0.5 ng/ml for Compound B and utilizing the relevant Compound A parameters, a required steady-state flux of 0.23 μg/cm²/hr was calculated for a skin area of 20 cm².

A further approach to estimating human plasma levels following transdermal administration was outlined by Howes et al. (ATLA, 24:81-106, 1996) and was based on a mass balance approach whereby the input and output quantities of drug are considered equal. Rewriting “input=output” for transdermal systems gives:

A×Kₚ×Cₐₚₚₗ=CL×Cₚ Eqn. 10.6

where A is the area of application, Kₚ is the permeability coefficient of the drug through the skin, Cₐₚₚₗ is the applied concentration, CL is the plasma clearance and Cₚ is the steady-state plasma concentration of the drug. Rearranging the equation to estimate Cₚ gives:

$${C}_{p} = \frac{A \cdot {K}_{p} \cdot {C}_{appl}}{CL} \qquad Eqn . 10.7$$

Inserting, a value of 20 cm² for “A”, a value of 5 mg/cm³ for “Cₐₚₚₗ” and the known CL value for D-2-Nal-c(Cys-Tyr-D-Trp-Lys-Val-Cys)-Thr-NH₂ for a 70 Kg human (292 (ml/hr)/Kg×70 Kg=20,440 cm³/hr) gives:

Cₚ=4.89×10−3(mg/hr/cm⁻⁴)Kp Eqn. 10.8

A number of methods for determining Kₚ based on the lipophilicity and molecular weight of the drug in question are available. Potts and Guy (Pharm. Res., 9:663-669, 1992) found the following relationship:

Log Kₚ(cm/hr)=−2.7+0.71 log KOW−0.0061MW Eqn. 10.9

while Morimoto et al. (1992) found that:

Kₚ(cm/s)=1.17×10−7KOW0.751+2.73×10−8 Eqn. 10.10

Another method of calculating Kₚ which makes use of in vitro data is described by Hewitt et al. (In vitro cutaneous disposition of a topical diclofenac lotion in human skin: Effect of a multidose regimen, in Percutaneous Absorption, Bronaugh, R. L. and Maibach, H. I. (Eds.), Marcel Dekker Inc., New York, 1999):

Kₚ(cm/h)=absorption(μg)/[area×concentration(μg/cm³)×time] Eqn. 10.11

Values for Kₚ were calculated using Eqns. 10.9 and 10.10 using the MW value for Compound B di-oleate (1,767 g/mol) and its previously determined KOW value (340.43). Additionally, Eqn. 10.11 was used to predict Kₚ using the in vitro value for absorption obtained across pig epidermis for the optimized donor vehicle. Table 21 shows the different Kₚ values obtained using all three equations.

TABLE 21

| Equation | Kₚ Value Obtained (cm/hr) |
|---|---|
| 10.9 | 2.085 × 10⁻¹² |
| 10.10 | 3.348 × 10⁻² |
| 10.11 | 3.476 × 10⁻⁴ |

Equations 10.9 and 10.10 were derived mainly on the basis of data for small water-soluble drugs. As such, the more relevant value was taken to be that derived from Eqn. 10.11. Returning to Eqn. 10.8, this gives Cₚ=(4.89×10−3)(3.476×10−4) mg/cm³ or a steady-state plasma level of 1.6998×10−6 mg/ml or 1.6998 ng/ml.

1-67. (canceled)

68. A fatty acid salt of a peptide, wherein said peptide is a somatostatin type-1, type-2, type-3, type-4 or type-5 receptor analog, or any combination thereof.

69. The fatty acid salt of a peptide according to claim 68, wherein said somatostatin receptor analog is a somatostatin type-2 receptor selective agonist.

70. The fatty acid salt of a peptide according to claim 68, wherein said somatostatin receptor analog is a somatostatin type-2 receptor agonist of:
D-2-Nal-c(Cys-Tyr-D-Trp-Lys-Val-Cys)-Thr-NH₂;
D-Phe-c(Cys-Phe-D-Trp-Lys-Thr-Cys)-Thr-ol;
[4-(2-hydroxyethyl)]-1-piperazinylacetyl-D-Phe-c(Cys-Tyr-D-Trp-Lys-Abu-Cys)-Thr-NH₂;
c(Tic-Tyr-D-Trp-Lys-Abu-Phe);
4-(2-Hydroxyethyl)-1-piperazine-2-ethanesulfonyl-D-Phe-c(Cys-Tyr-D-Trp-Lys-Abu-Cys)-Thr-NH₂;
[4-(2-hydroxyethyl)]-1-piperazinylacetyl-D-Phe-c(Cys-Tyr-D-Trp-Lys-Abu-Cys)-Thr-NH₂;
D-6-propyl-8beta-ergolinylmethyl-thioacetyl-D-Lys(D-6-propyl-8beta-ergolinyl-methylthioacetyl)-c(Cys-Tyr-D-Trp-Lys-Abu-Cys)-Thr-NH₂;
D-Cpa-c(Cys-Tyr-D-Trp-Lys-Val-Cys)-Thr-NH₂;
D-Phe-Cpa-Tyr-D-Trp-Lys-Val-Phe-Thr-NH₂;
D-Phe-Cpa-Tyr-D-Trp-Lys-Thr-Phe-Thr-NH₂; or
Ac-c(Cys-Lys-Asn-Cpa-Phe-D-Trp-Lys-Thr-Phe-Thr-Ser-D-Cys)-NH_{2.}

71. The fatty acid salt of a peptide according to claim 70, wherein said somatostatin type-2 receptor agonist is a di-oleate fatty acid salt of:
D-2-Nal-c(Cys-Tyr-D-Trp-Lys-Val-Cys)-Thr-NH₂;
D-Phe-c(Cys-Phe-D-Trp-Lys-Thr-Cys)-Thr-ol; or
[4-(2-hydroxyethyl)]-1-piperazinylacetyl-D-Phe-c(Cys-Tyr-D-Trp-Lys-Abu-Cys)-Thr-NH₂.

72. The fatty acid salt of a peptide according to claim 68, wherein said fatty acid is octanoic acid, nonanoic acid, decanoic acid, dodecanoic acid, tetradecanoic acid, cis-9-octadecanoic acid or cis,cis-9,12-octadecanoic acid.

73. The fatty acid salt of a peptide according to claim 72, wherein said fatty acid is cis-9-octadecanoic acid.

74. A composition comprising the fatty acid salt of a peptide according to claim 68 and a carrier suitable for transdermal delivery.

75. The composition according to claim 74, further comprising a chemical penetration enhancer.

76. The composition according to claim 75, wherein said chemical penetration enhancer is dimethylsulfoxide, dimethylformamide, dimethylacetamide, decanol, dodecanol, oleic acid, 1,8-cineole, propylene glycol, or sodium lauryl sulfate.

77. A transdermal delivery device comprising the fatty acid salt of a peptide according to claim 68.

78. A method of altering the bioavailability of a peptide comprising increasing the lipophilicity of said peptide, wherein said increasing comprises preparing a fatty acid salt of said peptide according to claim 68.


SEQUENCE LISTING (41 sequences)

SEQ ID NO: 1 - protein, 4 aa, Artificial Sequence
  synthetic anti-hemopoietic peptide
SDKP

SEQ ID NO: 2 - protein, 14 aa, Artificial Sequence
  synthetic bombesin peptide analog
EQSLGNQWARGHFM

SEQ ID NO: 3 - protein, 10 aa, Artificial Sequence
  synthetic bombesin peptide analog
GNQWARGHFM

SEQ ID NO: 4 - protein, 11 aa, Artificial Sequence
  synthetic chemokine peptide analog
CLDPKQKWIQC

SEQ ID NO: 5 - protein, 5 aa, Artificial Sequence
  synthetic chemokine peptide analog
CWIQC

SEQ ID NO: 6 - protein, 6 aa, Artificial Sequence
  synthetic chemokine peptide analog
GKWIQE

SEQ ID NO: 7 - protein, 28 aa, Artificial Sequence
  synthetic peptide ghrelin analog
GXEFLSPEHQRVQQRKESKKPPAKLQPR

SEQ ID NO: 8 - protein, 30 aa, Artificial Sequence
  synthetic peptide analog of ghrelin
HXEGTFTSDVSSYLEGQAAKEFIAWLVKXR

SEQ ID NO: 9 - protein, 30 aa, Artificial Sequence
  synthetic peptide analog of ghrelin
HSEGTFTSDVSSYLEGQAAKEFIAWLVKXR

SEQ ID NO: 10 - protein, 30 aa, Artificial Sequence
  synthetic peptide analog of ghrelin
HXEGTFTSDVSSYLEGQAAKEFIAWLVRXR

SEQ ID NO: 11 - protein, 34 aa, Artificial Sequence
  synthetic peptide analog of parathyroid hormone releasing hormone
AVSEHQLLHDKGKSIQDLRRRELLEKLLXKLHTA

SEQ ID NO: 12 - protein, 38 aa, Artificial Sequence
  synthetic peptide analog of neuropeptide Y
GXYPSKPDNPGEDAPAXDMARYYSALRHYINLITRPRY

SEQ ID NO: 13 - protein, 43 aa, Homo sapiens
  MISC_FEATURE (1)..(23): cyclic
CHSRSADVDSLSESSPNSSPGPCPEKAPPPQKPSHEGSYLLQP

SEQ ID NO: 14 - protein, 8 aa, Artificial Sequence
  synthetic urotensin II peptide analog
DCFWKYCV

SEQ ID NO: 15 - protein, 43 aa, Artificial Sequence
  Synthetic analog of glucose-dependent insulinotropic polypeptide (GIP)
YGEGTFISDYXIAXDKIHQQDFVNWLLAQKGKKNDWKHNITQH

SEQ ID NO: 16 - protein, 42 aa, Artificial Sequence
  Synthetic analog of glucose-dependent insulinotropic polypeptide (GIP)
YAEGTFISDYXIAMDKIHQQDFVNWLLAQKGKKNDWKHNIXQ

SEQ ID NO: 17 - protein, 42 aa, Artificial Sequence
  Synthetic analog of glucose-dependent insulinotropic polypeptide (GIP)
YAEGTFISDYXIAMDKIHQQDFVNWLLAQKGKKNDWKHNXTQ

SEQ ID NO: 18 - protein, 43 aa, Artificial Sequence
  Synthetic analog of glucose-dependent insulinotropic polypeptide (GIP)
YAEGTFISDYXIAMDKIHQQDFVNWLLAQKGKKNDWKHNITQH

SEQ ID NO: 19 - protein, 43 aa, Artificial Sequence
  Synthetic analog of glucose-dependent insulinotropic polypeptide (GIP)
YAEGTFISDYXIAMDKIHQQDFVNWLLAQKGKKNDWKHNITQN

SEQ ID NO: 20 - protein, 43 aa, Artificial Sequence
  Synthetic analog of glucose-dependent insulinotropic polypeptide (GIP)
YAEGTFISDYSIXMDKIHQQDFVNWLLAQKGKKNDWKHNITQD

SEQ ID NO: 21 - protein, 42 aa, Artificial Sequence
  Synthetic analog of glucose-dependent insulinotropic polypeptide (GIP)
YAEGTFISDYSIXXDKIHQQDFVNWLLAQKGKKNDWKHNXTQ

SEQ ID NO: 22 - protein, 42 aa, Artificial Sequence
  Synthetic analog of glucose-dependent insulinotropic polypeptide (GIP)
YAEGTFISDYSIXMDKIHQQDFVNWLLAQKGKKNDWKHNXTQ

SEQ ID NO: 23 - protein, 43 aa, Artificial Sequence
  Synthetic analog of glucose-dependent insulinotropic polypeptide (GIP)
YAEGTFISDYXIAXDKIHQQDFVNWLLAQKGKKNDWKHNITQH

SEQ ID NO: 24 - protein, 42 aa, Artificial Sequence
  Synthetic analog of glucose-dependent insulinotropic polypeptide (GIP)
YXEGTFISDYXIAXDKIHQQDFVNWLLAQKGKKNDWKHNITQ

SEQ ID NO: 25 - protein, 42 aa, Artificial Sequence
  Synthetic analog of glucose-dependent insulinotropic polypeptide (GIP)
YXEGTFISDYXIAMDKIHQQDFVNWLLAQKGKKNDWKHNIXQ

SEQ ID NO: 26 - protein, 42 aa, Artificial Sequence; identical to SEQ ID NO: 24
  Synthetic analog of glucose-dependent insulinotropic polypeptide (GIP)

SEQ ID NO: 27 - protein, 43 aa, Artificial Sequence
  Synthetic analog of glucose-dependent insulinotropic polypeptide (GIP)
YAEGTFISDYXIAMDKIHQQDFVNWLLAQKGKKNDWKHNITQK

SEQ ID NO: 28 - protein, 43 aa, Artificial Sequence
  Synthetic analog of glucose-dependent insulinotropic polypeptide (GIP)
YXEGTFISDYXIAXDKIHQQDFVNWLLAQKGKKNDWKHNITQC

SEQ ID NO: 29 - protein, 43 aa, Artificial Sequence; identical to SEQ ID NO: 28
  Synthetic analog of glucose-dependent insulinotropic polypeptide (GIP)

SEQ ID NO: 30 - protein, 30 aa, Artificial Sequence
  Synthetic analog of glucose-dependent insulinotropic polypeptide (GIP)
YXEGTFXSDYSIAMDKIHQQDFVNWLLAQK

SEQ ID NO: 31 - protein, 42 aa, Artificial Sequence
  Synthetic analog of glucose-dependent insulinotropic polypeptide (GIP)
YXEGTFISDYSIXXDKIHQQDFVNWLLAQKGKKNDWKHNITQ

SEQ ID NO: 32 - protein, 43 aa, Artificial Sequence; identical to SEQ ID NO: 27
  Synthetic analog of glucose-dependent insulinotropic polypeptide (GIP)

SEQ ID NO: 33 - protein, 43 aa, Artificial Sequence
  Synthetic analog of glucose-dependent insulinotropic polypeptide (GIP)
YAEGTFISDYXIAMDKIHQQDFVNWLLAQKGKKNDWKHNIXQC

SEQ ID NO: 34 - protein, 43 aa, Artificial Sequence
  Synthetic analog of glucose-dependent insulinotropic polypeptide (GIP)
XXEGTFISDYXIAMDKIHQQDFVNWLLAQKGKKNDWKHNITQH

SEQ ID NO: 35 - protein, 37 aa, Artificial Sequence
  Synthetic analog of glucose-dependent insulinotropic polypeptide (GIP)
XSDYSIAMDKIHQQDFVNWLLAQKGKKNDWKHNITQQ

SEQ ID NO: 36 - protein, 41 aa, Artificial Sequence
  Synthetic analog of glucose-dependent insulinotropic polypeptide (GIP)
XEGTFISDYSIXMDKIHQQDFVNWLLAQKGKKNDWKHNITQ

SEQ ID NO: 37 - protein, 43 aa, Artificial Sequence; identical to SEQ ID NO: 28
  Synthetic analog of glucose-dependent insulinotropic polypeptide (GIP)

SEQ ID NO: 38 - protein, 43 aa, Artificial Sequence; identical to SEQ ID NO: 28
  Synthetic analog of glucose-dependent insulinotropic polypeptide (GIP)

SEQ ID NO: 39 - protein, 15 aa, Artificial Sequence
  synthetic apoptotic control peptide analog
LSECLKRIGDELDSN

SEQ ID NO: 40 - protein, 15 aa, Artificial Sequence
  synthetic apoptotic control peptide analog
LSESLKRIGDELDSN

SEQ ID NO: 41 - protein, 43 aa, Artificial Sequence; identical to SEQ ID NO: 33
  Synthetic analog of glucose-dependent insulinotropic polypeptide (GIP)